(19) United States
(12) Patent Application Publication
(10) Pub. No.: US 2025/0205262 A1
(43) Pub. Date: Jun. 26, 2025

(54) SPHINGOLIPID-LOADED NANOBIOLOGICS FOR IMMUNE REGULATION

(71) Applicants: ICAHN SCHOOL OF MEDICINE AT MOUNT SINAI, New York, NY (US); STICHTING RADBOUD UNIVERSITAIR MEDISCH CENTRUM, Nijmegen (NL)

(72) Inventors: Abraham J. Teunissen, New York, NY (US); Willem J. Mulder, New York, NY (US); Raphaël Duivenvoorden, Nijmegen (NL); Zahi A. Fayad, New York, NY (US)

(21) Appl. No.: 18/850,960

(22) PCT Filed: Mar. 30, 2023
(86) PCT No.: PCT/US2023/065166

§ 371 Date: Sep. 25, 2024

Related U.S. Application Data
(60) Provisional application No. 63/326,191, filed on Mar. 31, 2022.

(51) Int. Cl. A61K 31/7032; A61K 9/107; A61K 9/51; A61K 47/69; A61P 35/00

(52) U.S. Cl. CPC A61K 31/7032; A61K 9/107; A61K 9/5123; A61K 9/5169; A61K 47/6917; A61P 35/00

(57) ABSTRACT

Provided herein are sphingolipid-loaded nanobiologics comprising: an apolipoprotein A-1 (apoA-1) or a peptide mimetic of apoA-1; a sphingolipid; and cholesterol, and uses thereof e.g., a method of innate immune regulation including stimulating a trained immunity response in a subject, and a method of treatment of cancer, comprising administering a nanobiologic composition.

This application claims priority to U.S. Provisional Application No. 63/326,191, filed on Mar. 31, 2022, the contents of which is hereby incorporated by reference in its entirety for all purposes.

### STATEMENT OF GOVERNMENT INTEREST

This invention was made in part with government support under Grant Nos. R01 CA220234, R01 HL144072, and P01 HL131478 awarded by the National Institute of Health. The United States Government has certain rights in this invention.

### DESCRIPTION OF THE TEXT FILE SUBMITTED ELECTRONICALLY

This application contains a Sequence Listing that has been submitted electronically as an XML file named 27527-0207WO1_SL_ST26.xml. The XML file, created on Mar. 30, 2023, is 490,269 bytes in size. The material in the XML file is hereby incorporated by reference in its entirety.

### BACKGROUND

The immune system plays an essential role in the pathophysiology of major diseases such as atherosclerosis, diabetes, and cancer. However, most of the immunotherapy strategies currently being developed focus on either effector molecules, such as cytokines, or T lymphocytes, which are cells from the adaptive immune system. In autoimmune and autoinflammatory diseases, anti-cytokine therapies can successfully neutralize bioactive cytokines, while the most intensely used immunotherapy in cancer patients comprises the application of checkpoint-inhibitor drugs. Though the innate immune system was long believed to lack memory, recent studies show that innate immune cells undergo metabolic and epigenetic rewiring, adjusting their functional programs in a process termed ‘trained immunity’.

Sphingolipids are an extended family of bioactive lipids that are universally present in eukaryotes and possess immunomodulatory properties. These lipid molecules regulate basic cellular processes, including growth, adhesion, migration, apoptosis, and senescence. In addition, sphingolipids play a critical role in shaping immune responses.

A need exists for therapeutic agents, and compositions thereof that engage the innate immune system, and methods of use thereof.

### SUMMARY

The present disclosure is directed to sphingolipid-loaded nanobiologics and methods of use thereof, e.g., for the treatment of cancer.

In embodiments, the present disclosure provides a nanobiologic composition comprising:
(a) a apolipoprotein A-I (apoA-I) or a peptide mimetic of apoA-I; and
(b) a sphingolipid.

In embodiments, the present disclosure provides a nanobiologic composition comprising:
(a) a apolipoprotein A-I (apoA-I) or a peptide mimetic of apoA-I;
(b) a sphingolipid; and
(d) cholesterol; and
wherein the composition is a nanoparticle having a diameter between about 8 nm and about 150 nm. In embodiments, the nanobiologic composition further comprises a phospholipid.

In embodiments, the present disclosure provides a nanobiologic composition comprising:
(a) a apolipoprotein A-I (apoA-I) or a peptide mimetic of apoA-I;
(b) a sphingolipid
(c) a phospholipid; and
(d) cholesterol;
wherein the sphingolipid is present as about 1-100 mol % of the total lipid composition of the nanobiologic composition; and
wherein the composition is a nanoparticle having a diameter between about 8 nm and about 150 nm.

In embodiments, the sphingolipid is selected from the group consisting of a ceramide, sphingomyelin, dihydroceramide, glucosylceramide, sphingosine, sphingosine-1-phosphate, galactosylceramide, ceramide-1-phosphate, lactosylceramide, and mixtures thereof.

In embodiments, the sphingolipid is of Formula (I):

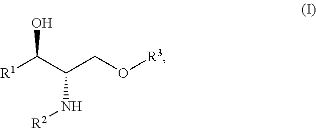

or a pharmaceutically acceptable salt thereof, wherein:
R¹ is a saturated aliphatic chain or an unsaturated aliphatic chain;
R² is H, or R⁴, or C(O)R⁴;
R³ is H, a sugar, P(O)(OH)₂, or P(O)(OH)O(CH₂)ₙR⁵ where n=1-10;
R⁴ is a saturated aliphatic chain or an unsaturated aliphatic chain; and
R⁵ is H, NH₂, N(CH₃)₃⁺, OH, or a sugar.

In embodiments, the sphingolipid is of Formula (I-A):

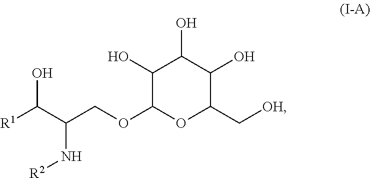

wherein R¹ and R² are defined herein.

In embodiments, the sphingolipid is of Formula (II):

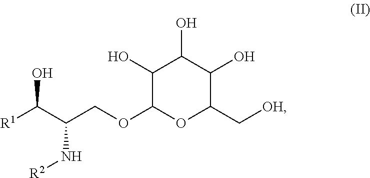

wherein R¹ and R² are defined herein.

In embodiments, the sphingolipid is a of Formula (II-A):

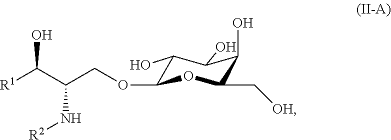

wherein R¹ and R² are defined herein.

In embodiments, the sphingolipid of Formula (II) is a sphingolipid of Formula (II-B):

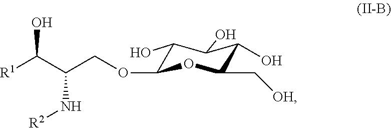

wherein R¹ and R² are defined herein.

In embodiments, the nanobiologic composition comprises human apolipoprotein A-I (apoA-I).

In embodiments, the nanobiologic composition is discoidal in shape.

In embodiments, the nanobiologic composition is spherical in shape.

In embodiments, the nanobiologic composition is suitable for intravenous or intra-arterial administration.

In embodiments, the present disclosure provides methods for treating a cancer in a subject in need thereof, comprising administering to the subject an effective amount of a nanobiologic composition provided herein.

In embodiments, the present disclosure provides methods for treating atherosclerosis in a subject in need thereof, comprising administering to the subject an effective amount of a nanobiologic composition provided herein.

In embodiments, the present disclosure provides methods for the prophylaxis of organ or tissue rejection in a subject in need thereof, comprising administering to the subject an effective amount of a nanobiologic composition provided herein.

### DESCRIPTION OF THE FIGURES

FIG. 1A is a schematic depiction of the predominant sphingolipid classes and their metabolic interconversion. Yellow boxes indicate sphingolipid classes that were incorporated in the nanobiologics.

FIG. 1B is a schematic depiction of the in vitro trained immunity assay performed using the sphingolipid-loaded nanobiologics.

FIG. 1C is a schematic depiction of a sphingolipid-loaded nanobiologic composition of the present disclosure.

FIG. 1D and FIG. 1E are graphs depicting the results of the assay described in Example 2 in which PBMCs were stimulated for 24 hours with sphingolipid-loaded nanobiologics prepared in Example 1 alone (FIG. 1D) or in combination with HKCA (FIG. 1E). After a five-day resting period, cells were restimulated with LPS for 24 hours and cytokine production measured in the supernatant by ELISA (n=3). Data are expressed as log 2 fold change compared to untrained (RPMI) PBMCs in (FIG. 1D) or compared to HKCA-trained PBMCs in (FIG. 1E). p-values were calculated using a one-way ANOVA with Dunnett's post-test.

FIG. 2 shows tumor growth curves in a B16F10 mouse melanoma model following treatment with PBS or nanobiologic compositions described in Example 3.

FIG. 3A shows (A) Lactate dehydrogenase (LDH) measurement of PBMCs treated with sphingolipid-nanobiologics for 24 hours.

FIG. 3B-3E show cytokine production measured in the supernatant (n=3) after PBMCs were stimulated for 24 hours with sphingolipid-nanobiologics alone (FIG. 3B, FIG. 3C) or in combination with heat-killed Candida Albicans (HKCA) (FIG. 3D, FIG. 3E) and restimulated after a five-day resting period with LPS for 24 hours. Data are expressed as fold change compared to untrained (RPMI) PBMCs in (B, C) or compared to HKCA-trained PBMCs in (D, E). * p<0.05, ** p<0.01, *** p<0.001. p-values were calculated using one-way ANOVA with Dunnett's post-test.

FIG. 4 shows blood chemistry analyses performed in the study described in Example 2. ALP=alkaline phosphatase, AST=aspartate aminotransferase, ALT=alanine transaminase, BUN=blood urea nitrogen, n=4.

FIG. 5 shows pharmacokinetics of ⁸⁹Zr radiolabeled nanobiologic formulation #13.

FIG. 6 shows biodistribution at 24 hours by ex vivo gamma counting, (n=5) of ⁸⁹Zr radiolabeled nanobiologic formulation #13.

FIG. 7 shows bone marrow analyzed by flow cytometry after 24 hours following i.v. administration of nanobiologic formulation #13 loaded with the lipophilic fluorophore DiOC18 to B16F10 melanoma mouse model (n=5).

### DEFINITIONS

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “conservative substitution,” as used herein, refers to the exchange of one amino acid for another among the following amino acid groups: (i) the aliphatic amino acids (alanine, valine, leucine, and isoleucine); (ii) amino acids with hydroxyl groups (serine and threonine); (iii) acidic amino acids (glutamic acid and aspartic acid); (iv) amino acids with amide side chains (asparagine and glutamine); (v) basic amino acids (lysine and arginine); (vii) amino acids with aromatic side chains (phenylalanine, tyrosine, and tryptophan).

“Salts” include those obtained by reacting a compound functioning as a base, with an inorganic or organic acid to form a salt, or those obtained by reacting a compound functioning as an acid, with an inorganic or organic base to form a salt. “Salts” include derivatives of an active agent, wherein the active agent is modified by making acid or base addition salts thereof. Preferably, the salts are pharmaceutically acceptable salts. Such salts include, but are not limited to, pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, 3-(4-hydroxybenzoyl)benzoic acid, cinnamic, citric, fumaric, glycolic, lactic, maleic, malic, malonic, mandelic, oxalic, picric, pyruvic, salicylic, succinic, methanesulfonic, ethanesulfonic, 2-hydroxyethanesulfonic acid, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, 4-chlorobenzenesulfonic acid, 2-naphthalenesulfonic acid, camphorsulfonic acid, p-toluenesulfonic acids, sulphates, nitrates, phosphates, perchlorates, borates, acetates, benzoates, hydroxynaphthoates, glycerophosphates, ketoglutarates and the like. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Standard methods for the preparation of pharmaceutically acceptable salts and their formulations are well known in the art, and are disclosed in various references, including for example, “Remington: The Science and Practice of Pharmacy”, A. Gennaro, ed., 20th edition, Lippincott, Williams & Wilkins, Philadelphia, PA.

The term “carrier” or “vehicle” as used interchangeably herein encompasses carriers, excipients, adjuvants, and diluents or a combination of any of the foregoing, meaning a material, composition or vehicle, such as a liquid or solid filler, diluent, excipient, solvent or encapsulating material involved in carrying or transporting a pharmaceutical agent from one organ, or portion of the body, to another organ or portion of the body. In addition to the adjuvants, excipients and diluents known to one skilled in the art, the carrier includes nanoparticles of organic and inorganic nature.

The term “treating” as used herein with regard to a patient, refers to improving at least one symptom of the patient's disease or disorder. Treating can be improving, or at least partially ameliorating a disease or disorder.

The term “patient” or “subject” as used herein, includes all mammals and more particularly includes humans. The methods described herein may be useful for both human therapy and veterinary applications. In embodiments, the subject is a human.

As used herein, “therapeutically effective amount” means the amount of a compound or a therapeutically active agent that, when administered to a subject for treating a disease or other undesirable medical condition, is sufficient to have a beneficial effect with respect to that disease or condition. The therapeutically effective amount will vary depending on the type of the selected compound or a therapeutically active agent, the disease or condition and its severity, and the age, weight, etc. of the patient to be treated.

The term “aliphatic” or “aliphatic group”, or “aliphatic chain” as used herein, means a straight-chain (i.e., unbranched), branched, or cyclic, substituted or unsubstituted hydrocarbon chain that is completely saturated or that contains one or more units of unsaturation and has a single point of attachment to the rest of the molecule. Unless otherwise specified, aliphatic groups contain 1-30 aliphatic carbon atoms. In embodiments, aliphatic groups contain 10-30 aliphatic carbon atoms. In embodiments, aliphatic groups contain 10-20 aliphatic carbon atoms. In embodiments, aliphatic groups contain 15-20 aliphatic carbon atoms. In embodiments, aliphatic groups contain 15-17 aliphatic carbon atoms. In embodiments, aliphatic groups contain 1-10 aliphatic carbon atoms. In embodiments, aliphatic groups contain 1-8 aliphatic carbon atoms. In embodiments, aliphatic groups contain 1-6 aliphatic carbon atoms, in embodiments aliphatic groups contain 1-4 aliphatic carbon atoms. Aliphatic groups may be saturated or unsaturated, linear or branched, substituted or unsubstituted alkyl, alkenyl, or alkynyl groups.

The term “triglyceride” as used herein means an ester derived from glycerol and three fatty acids. The fatty acids may be the same or different. The notation used in this specification to describe a triglyceride is the same as that used below to describe a fatty acid. Fatty acids can attach to the glycerol molecule in any order, e.g., any fatty acid can react with any of the hydroxyl groups of the glycerol molecule for forming an ester linkage. For example. In a non-limiting example, a triglyceride can comprise glycerol with any combination of the following fatty acids: C18:1, C14:1, C16:1, polyunsaturated, and saturated. A triglyceride of C18:1 fatty acid simply means that the fatty acid components of the triglyceride are derived from or based upon a C18:1 fatty acid. That is, a C18:1 triglyceride is an ester of glycerol and three fatty acids of 18 carbon atoms each with each fatty acid having one double bond. Similarly, a C14:1 triglyceride is an ester of glycerol and three fatty acids of 14 carbon atoms each with each fatty acid having one double bond. Likewise, a C16:1 triglyceride is an ester of glycerol and three fatty acids of 16 carbon atoms each with each fatty acid having one double bond. Triglycerides of C18:1 fatty acids in combination with C14:1 and/or C16:1 fatty acids means that: (a) a C18:1 triglyceride is mixed with a C14:1 triglyceride or a C16:1 triglyceride or both; or (b) at least one of the fatty acid components of the triglyceride is derived from or based upon a C18:1 fatty acid, while the other two are derived from or based upon C14:1 fatty acid and/or C16:1 fatty acid.

The term “fatty acid” and like terms mean a carboxylic acid with a long aliphatic tail that is either saturated or unsaturated. The term “long aliphatic tail” and “fatty acid chain” are used interchangeably herein. As used herein, the fatty acid chain length includes from C4 to C30 (e.g., C6 to C30), saturated or unsaturated, cis or trans, Zusammen (Z) or entgegen (E), unsubstituted or substituted with C1-10 side chains.

Unsaturated fatty acids have one or more double bonds between carbon atoms. Saturated fatty acids do not contain any double bonds. In embodiments, a fatty acid may be described herein by the capital letter “C” for carbon atom, followed by a number describing the number of carbon atoms in the fatty acid, followed by a colon and another number for the number of double bonds in the fatty acid. For example, C16:1 denotes a fatty acid of 16 carbon atoms with one double bond, e.g., palmitoleic acid. The number after the colon in this notation neither designates the placement of the double bond(s) in the fatty acid nor whether the hydrogen atoms bonded to the carbon atoms of the double bond are cis to one another. Other examples of this notation include C18:0 (stearic acid), C18:1 (oleic acid), C18:2 (linoleic acid), C18:3 (α-linolenic acid) and C20:4 (arachidonic acid).

The term “sterols” as used herein refers to animal or plant steroids which contain at least one hydroxyl group. In embodiments, the sterols of the present disclosure have a single hydroxyl group at the C3-position. In general, sterols contain 27 to 30 carbon atoms and one double bond in the 5/6 position and occasionally in the 7/8, 8/9 or other positions. Non-limiting examples of sterols contemplated herein, include sigmasterol, campesterol, sitosterol, sitostanols, brassicasterols, stigmasterol, D5 avenasterol, D7 avenasterol, ergosterol, citrostadienol, cholesterol, lanosterols, spongosterols, fungisterols, stellasterols, zymosterols and mixtures thereof. In embodiments, the sterol is cholesterol.

The term “sterol ester” as used herein refers to an ester derived from a sterol as defined above and a carboxylic acid. In embodiments, the carboxylic acid is of the formula Rb—C(O)—OH wherein Rb is an aliphatic group. In embodiments, the carboxylic acid is acetic acid, propionic acid, hexanoic acid, butyric acid, valeric acid, caproic acid, caprylic acid, 2-ethyl hexanoic acid, capric acid, cyclopentanepropionic acid, lauric acid, isotridecanoic acid, myristic acid, palmitic acid, palmitoleic acid, stearic acid, isostearic acid, oleic acid, elaidic acid, petroselic acid, linoleic acid, conjugated linoleic acid (CLA), linolenic acid, elaeosteric acid, arachic acid, arachidonic acid, gadoleic acid, behenic acid and erucic acid.

The term “phospholipid” refers to an amphiphilic compound comprised of a glycerol molecule bound to two fatty acids “tails” and a phosphate “head” group. The phosphate group may be further bound to hydrogen, choline, serine, ethanolamine, or inositol, thus, diversifying into phosphatidic acid, phosphatidylcholine, phosphatidylserine, phosphatidylethanolamine, and phosphatidylinositol phospholipids, respectively.

The term “lysophospholipid,” as used herein, refers to a derivative of a phospholipid (e.g., as defined above) in which one of the acyl fatty acid tails has been removed by hydrolysis. Thus, lysophospholipids have a free alcohol in either the sn-1 or sn-2 position. In non-limiting embodiments, the lysophospholipid is 1-myristoyl-2-hydroxy-sn-glycero-3-phosphocholine (MHPC), 1-palmitoyl-2-hydroxy-sn-glycero-3-phosphocholine (PHPC) or 1-stearoyl-2-hydroxy-sn-glycero-3-phosphocholine (SHPC).

The term “apolipoprotein A-I” or “apoA-I”, and also “apolipoprotein A1” or “apoA1”, refers to a protein that is encoded by the APOAI gene in humans.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

Until recently, immunologic memory was considered an exclusive feature of the adaptive immune system. However, this view has been challenged by the discovery that innate immune cells also adapt and harness a form of immunologic memory. After exposure to certain stimuli, monocytes and macrophages can develop a quicker and stronger inflammatory response to subsequent unrelated stimuli.

Innate immune memory, also called “trained immunity,” is a metabolically and epigenetically regulated functional state of myeloid cells. Trained immunity is important for host defense, but can also be modulated to achieve therapeutic goals. For example, inhibiting trained immunity can be used to promote organ acceptance after transplant and promoting trained immunity can be used to provide an anti-cancer effect.

The epigenetic modifications underlying trained immunity are closely linked to metabolic changes, including the induction of aerobic glycolysis, glutaminolysis and cholesterol metabolism. Intermediates of these metabolic pathways serve as substrates for epigenetic enzymes or as regulators of these enzymes.

### Nanobiologic Compositions

Nanobiologics may contain multiple components including lipids (e.g., phospholipids, sphingolipids, triglycerides, cholesterol) and apolipoprotein A (apoA-1), the main protein constituent of high-density lipoprotein.

In embodiments, the nanobiologic composition is spherical in shape. Spherical nanobiologic compositions typically comprise apoA-1 or a peptide mimetic of ApoA-1, one or more phospholipids, one or more sphingolipids, one or more therapeutically active agents (e.g., small molecule drug or prodrug thereof) optionally cholesterol, and a hydrophobic core material such as one or more triglycerides or one or more polymers. For example, the inclusion of one or more triglycerides and/or one or more polymers in the nanoparticles disclosed herein, may facilitate modulation of nanoparticle size (e.g., from about 10 nm to over 30 nm, or over 100 nm) and shape (from discoidal to spherical). In turn, the size, rigidity, and viscosity of the nanobiologic composition may also affect loading and biodistribution.

Without being bound by theory, nanobiologics' intrinsic propensity for myeloid cell uptake allows an efficient delivery of payloads to the innate immune system.

In embodiments, the present disclosure provides a nanobiologic composition comprising:
(a) a apolipoprotein A-I (apoA-I) or a peptide mimetic of apoA-I; and
(b) a sphingolipid;

In embodiments, the nanobiologic compositions of the present disclosure may further comprise one or more additional components (e.g., 1, 2, 3, or 4 components) independently selected for each occurrence from the group consisting of a phospholipid, a lysophospholipid, a hydrophobic matrix core molecule(s), and a sterol (e.g., cholesterol).

In embodiments, the present disclosure provides a nanobiologic composition comprising:
(a) a apolipoprotein A-I (apoA-I) or a peptide mimetic of apoA-I;
(b) a sphingolipid; and
(c) a sterol (e.g., cholesterol).

In embodiments, the present disclosure provides a nanobiologic composition comprising:
(a) a apolipoprotein A-I (apoA-I) or a peptide mimetic of apoA-I;
(b) a phospholipid; and
(c) a sphingolipid.

In embodiments, the present disclosure provides a nanobiologic composition comprising:
(a) a apolipoprotein A-I (apoA-I) or a peptide mimetic of apoA-I;
(b) a sphingolipid
(c) a phospholipid; and
(d) a lysophospholipid.

In embodiments, the present disclosure provides a nanobiologic composition comprising:
(a) a apolipoprotein A-I (apoA-I) or a peptide mimetic of apoA-I;
(b) a sphingolipid;
(c) a phospholipid; and
(d) a sterol (e.g., cholesterol). In embodiments, the present disclosure provides a nanobiologic composition comprising:
(a) a apolipoprotein A-I (apoA-I) or a peptide mimetic of apoA-I;
(b) a sphingolipid;
(c) phospholipid; and
(d) cholesterol;
wherein the composition is a nanoparticle having a diameter between about 8 nm and about 150 nm.

In embodiments, the present disclosure provides a nanobiologic composition comprising:
(a) a apolipoprotein A-I (apoA-I) or a peptide mimetic of apoA-I;
(b) a sphingolipid
(c) a phospholipid;
(d) a lysophospholipid; and
(e) a sterol (e.g., cholesterol).

In embodiments, the present disclosure provides a nanobiologic composition comprising:
(a) a apolipoprotein A-I (apoA-I) or a peptide mimetic of apoA-I;
(b) a phospholipid;
(c) cholesterol; and
(d) a sphingolipid
wherein the sphingolipid is present as about 1-100 mol % of the total lipid composition of the nanobiologic composition; and
the composition is a nanoparticle having a diameter between about 8 nm and about 150 nm.

In embodiments, the present disclosure provides a nanobiologic composition comprising:
(a) a apolipoprotein A-I (apoA-I) or a peptide mimetic of apoA-I;
(b) a sphingolipid;
(c) a sterol (e.g., cholesterol); and
(d) a hydrophobic matrix core (e.g., a triglyceride).

In embodiments, the present disclosure provides a nanobiologic composition comprising:
(a) a apolipoprotein A-I (apoA-I) or a peptide mimetic of apoA-I;
(b) a phospholipid;
(c) a sphingolipid; and
(d) a hydrophobic matrix core (e.g., a triglyceride).

In embodiments, the present disclosure provides a nanobiologic composition comprising:
(a) a apolipoprotein A-I (apoA-I) or a peptide mimetic of apoA-I;
(b) a sphingolipid
(c) a phospholipid;
(d) a lysophospholipid;
(e) a hydrophobic matrix core (e.g., a triglyceride).

In embodiments, the present disclosure provides a nanobiologic composition comprising:
(a) a apolipoprotein A-I (apoA-I) or a peptide mimetic of apoA-I;
(b) a sphingolipid;
(c) a phospholipid;
(d) cholesterol; and
(e) a hydrophobic matrix core (e.g., a triglyceride).

In embodiments, the present disclosure provides a nanobiologic composition comprising:
(a) a apolipoprotein A-I (apoA-I) or a peptide mimetic of apoA-I;
(b) a sphingolipid
(c) a phospholipid;
(d) a lysophospholipid;
(e) a sterol (e.g., cholesterol); and
(f) a hydrophobic matrix core (e.g., a triglyceride).

In embodiments of the nanobiologic compositions provided herein, the sphingolipid is selected from the group consisting of a ceramide, sphingomyelin, dihydroceramide, glucosylceramide, sphingosine, sphingosine-1-phosphate, galactosylceramide, ceramide-1-phosphate, lactosylceramide, and mixtures thereof.

In embodiments of the nanobiologic compositions provided herein, the sphingolipid is selected from the group consisting of a dihydroceramide, glucosylceramide, sphingosine, sphingosine-1-phosphate, galactosylceramide, ceramide-1-phosphate, lactosylceramide, and mixtures thereof.

In embodiments of the nanobiologic compositions provided herein, the sphingolipid is selected from the group consisting of a dihydroceramide, glucosylceramide, sphingosine, sphingosine-1-phosphate, galactosylceramide, ceramide-1-phosphate, lactosylceramide, and mixtures thereof.

In embodiments of the nanobiologic compositions provided herein, the sphingolipid is selected from the group consisting of a glucosylceramide, galactosylceramide, and a lactosylceramide.

In embodiments of the nanobiologic compositions provided herein, the sphingolipid is a glucosylceramide or a galactosylceramide.

In embodiments of the nanobiologic compositions provided herein, the sphingolipid is a ceramide. In embodiments, the sphingolipid is a sphingomyelin. In embodiments, the sphingolipid is a dihydroceramide. In embodiments, the sphingolipid is a glucosylceramide. In embodiments, the sphingolipid is a sphingosine. In embodiments, the sphingolipid is a sphingosine-1-phosphate. In embodiments, the sphingolipid is a galactosylceramide. In embodiments, the sphingolipid is a ceramide-1-phosphate. In embodiments, the sphingolipid is a lactosylceramide.

In embodiments of the nanobiologic compositions provided herein, the sphingolipid is selected from the group consisting of a ceramide, sphingomyelin, cerebroside, sulfatides, globoside, ganglioside and mixtures thereof.

In embodiments of the nanobiologic compositions provided herein, the sphingolipid is one or more sphingolipids selected from a ceramide, phytosphingosine, phosphosphingolipid, glycosphingolipid, or a sphingosine. In embodiments, the sphingolipid may be selected from the group consisting of a sphingosine, a sphinganine, a ceramide, a sphingomyelin, a ganglioside, a glycosphingolipid, a phosphosphingolipid, a phytosphingosine, and derivatives thereof.

In embodiments of the nanobiologic compositions provided herein, the sphingolipid is one or more sphingolipids selected from a phytoceramide, sulfatide, lactosyl sphingolipid, galactosyl sphingolipid, glucosyl sphingolipid, ganglioside, globoside, 2-hydroxy ceramide, dihydroceramide phosphate, dihydroceramide, 1-O-acyl ceramide, ceramide, methylated sphingosine, phosphorylated sphingosine, sphinganine. In embodiments, the sphingolipid is a natural sphingosine (e.g., egg sphingosine, brain sphingosine) or a natural ceramide (e.g., ceramide (egg), ceramide (brain), brain CPE).

In embodiments of the nanobiologic compositions provided herein, the sphingolipid is one or more sphingolipids selected from: D-ribo-Phytosphingosine-1-Phosphate, D-ribo-phytosphingosine (C17 base), Phytosphingosine-N,N-Dimethyl, N-24:0 (2S—OH) Phytosphingosine, Phytosphingosine-N,N-Dimethyl, N-02:0 Phytosphingosine, N-08:0 Phytosphingosine, N-18:0 Phytosphingosine, N-16:0 Phytosphingosine, N-24:0 Phytosphingosine, N-24:0 (2R—OH) Phytosphingosine, 24:0 CPE (d18:1/24:0), 24:1 CPE (d18/24:1), C12 Sphingosyl PE (d17:1/12:0), Sphingosyl PE (d18:1), Sphingosyl PI (d18:1), KRN7000, Sulfatides (Brain), C24:1 Mono-Sulfo Galactosyl(ß) Ceramide (d18:1/24:1), C17 Mono-Sulfo Galactosyl(ß) Ceramide (d18:1/17:0), C12 Mono-Sulfo Galactosyl(ß) Ceramide (d18:1/12:0), C12 Di-Sulfo Galactosyl(ß) Ceramide (d18:1/12:0), C24 Mono-Sulfo Galactosyl(ß) Ceramide (d18:1/24:0), 18:0 (2R—OH) Sulfo GalCer, 18:0 (2S—OH) Sulfo GalCer, C24:1 Mono-sulfo galactosyl (alpha) ceramide (d18:1/24:1), C8 Lactosyl(ß) Ceramide (d18:1/8:0), Lactosyl(ß) Sphingosine (d18:1), C12 Lactosyl(ß) Ceramide (d18:1/12:0), C8 L-threo-Lactosyl(ß) Ceramide (d18:1/8:0), C16 Lactosyl(ß) Ceramide (d18:1/16:0), C24 Lactosyl(ß) Ceramide (d18:1/24:0), C18:1 Lactosyl(ß) Ceramide (d18:1/18:1), C17 Lactosyl(ß) Ceramide (d18:1/17:0), C24:1 Lactosyl(ß) Ceramide (d18:1/24:1), C18 Lactosyl(ß) Ceramide (d18:1/18:0); C8 Galactosyl(α) Ceramide (d18:1/8:0), C16 Galactosyl(α) Ceramide (d18:1/16:0), C24:1 Galactosyl(α) Ceramide (d18:1/24:1 (15Z)), Galactosyl(α)Sphingsosine (d18:1), Adamantanyl Galactosyl(ß) Ceramide, C16 Galactosyl(ß) Ceramide (d18:1/16:0), Galactosyl(ß) Sphingosine (d18:1), C8 Galactosyl(ß) Ceramide (d18:1/8:0), C12 Galactosyl(ß) Ceramide (d18:1/12:0), C24:1 Galactosyl(ß) Ceramide (d18:1/24:1 (15Z)), Galactosyl(ß) Dimethyl Sphingosine (d18:1), C18:1 Galactosyl(ß) Ceramide (d18:1/18:1 (9Z)), C16 Galactosyl(α) Dihydroceramide (d18:0/16:0), C16 Galactosyl(ß) Dihydroceramide (d18:0/16:0), C18 (2R—OH) Galactosyl(ß) Ceramide, C18 (2S—OH) Galactosyl(ß) Ceramide, C18 Galactosyl(ß) Ceramide (d18:1/18:0), C24:0 Galactosyl(ß) ceramide (d18:1/24:0), Cerebrosides (Brain), Glucosylceramide (Soy), glucosyl(ß) Sphingosine (d20:1), Glucosyl(α) Sphingosine (d18:1), Glucosyl(ß) Sphingosine (d18:1), C16 Glucosyl(ß) Ceramide (d18:1/16:0), C8 Glucosyl(ß) Ceramide (d18:1/8:0), C12 Glucosyl(ß) Ceramide (d18:1/12:0), C18 Glucosyl(ß) Ceramide (d18:1/18:0), C18:1 Glucosyl(ß) Ceramide (d18:1/18:1 (9Z)), C24:1 Glucosyl(ß) Ceramide (d18:1/24:1 (15Z)), C17 Glucosyl(ß) Ceramide (d18:1/17:0), ganglioside-Total, Ganglioside GD1a (Porcine Brain), Ganglioside GD1b (Porcine Brain), Ganglioside GM3 (Bovine Milk), Ganglioside GT1b (Porcine Brain), Ganglioside GD3 (Bovine Milk), NGcGM3 (Bovine Spleen), Ganglioside GM1 (Ovine Brain), C18:0 GM3 (synthetic), C17:0 GD1a (d18:1/17:0), Ganglioside GQ1b (Porcine Brain), C20:0 GM1 (synthetic), C18:0 GT1b (d18:1/18:0), C18:0 GD1a (d18:1/18:0), C17:0 GM1 (synthetic), C18:0 GM1-Biotin (synthetic), C17:0 GB3 (synthetic), C17:0 GA2, Lyso iGB3 (synthetic), C17:0 iGB3 (synthetic), Lyso GB3 (synthetic), Lyso GA2, Natural Sphingomyelin (Egg SM, Brain SM, Milk SM), synthetic sphingomyelin derivatives (e.g., 02:0 SM (d18:1/2:0), 06:0 SM (d18:1/6:0), 12:0 SM (d18:1/12:0), 16:0 SM (d18:1/16:0), 17:0 SM (d18:1/17:0), 18:0 SM (d18:1/18:0), 18:1 SM (d18:1/18:1 (9Z)), 24:0 SM, 24:1 SM, Lyso SM (d18:1), Lyso SM (dihydro) (d18:0), Lyso SM-d7, Lyso SM (d17:1), 12:0 Dihydro SM (d18:0/12:0), 16:1 SM (d18:1/16:1 (9Z)), 14:0 SM (d18:1/14:0), 12:0 (2R—OH) Ceramide, 2:0 (2S—OH) Ceramide, 16:0 (2R—OH) Ceramide, 16:0 (2S—OH) Ceramide, 17:0 (2R—OH) Ceramide, 17:0 (2S—OH) Ceramide, 20:0 (2R—OH) Ceramide, 20:0 (2S—OH) Ceramide, 22:0 (2R—OH) Ceramide, 22:0 (2S—OH) Ceramide, 24:0 (2R—OH) Ceramide, 24:0 (2S—OH) Ceramide, 24:1 (2R—OH) Ceramide, 24:1 (2S—OH) Ceramide, 18:1 (2R—OH) Ceramide, 18:1 (2S—OH) Ceramide, 18:0 (2R—OH) Ceramide, 18:0 (2S—OH) Ceramide, C16 Dihydroceramide-1-Phosphate (d18:0/16:0), C24 Dihydroceramide-1-Phosphate (d18:0/24:0), C18:1 Dihydroceramide (d18:0/18:1 (9Z)), C2 Dihydroceramide (d18:0/2:0), C8 Dihydroceramide (d18:0/8:0), C18 Dihydroceramide (d18:0/18:0), C24 Dihydroceramide (d18:0/24:0), C24:1 Dihydroceramide (d18:0/24:1 (15Z)), C14 dihydroceramide (d18:0/14:0), C6 Dihydroceramide (d18:0/6:0), 6 Dihydroceramide (d18:0/16:0), C12 Dihydroceramide (d18:0/12:0), 1-O-Acyl-Ceramide, C24 Ceramide-1-Phosphate (d18:1/24:0), C2 Ceramide-1-Phosphate (d18:1/2:0), C12 Ceramide-1-Phosphate (d18:1/12:0), C8 Ceramide-1-Phosphate (d18:1/8:0), C16 Ceramide-1-Phosphate (d18:1/16:0), 16:0 3-deoxy-CIP, C18:1 Ceramide-1-Phosphate (d18:1/18:1 (9Z)), C8 Ceramide-1-Phosphate (d17:1/8:0), C12 ceramide-1,3-cyclic-phosphate (d18:1/12:0), C16 ceramide-1,3-cyclic-phosphate (d18:1/16:0), C6 ceramide-1,3-cyclic-phosphate (d18:1/6:0), C15 Ceramide (d18:1/15:0), C22 Ceramide (d18:1/22:0), C2 Ceramide (d18:1/2:0), C4 Ceramide (d18:1/4:0), C6 Ceramide (d18:1/6:0), C8 Ceramide (d18:1/8:0), C10 Ceramide (d18:1/10:0), C12 Ceramide (d18:1/12:0), C14 Ceramide (d18:1/14:0), C16 Ceramide (d18:1/16:0), C17 Ceramide (d18:1/17:0), C18 Ceramide (d18:1/18:0), C18:1 Ceramide (d18:1/18:1 (9Z)), C20 Ceramide (d18:1/20:0), C24 Ceramide (d18:1/24:0), C24:1 Ceramide (d18:1/24:1 (15Z)), C18 Ceramide (d17:1/18:0), C20 Ceramide (d17:1/20:0), C24 Ceramide (d17:1/24:0), C24:1 Ceramide (d17:1/24:1 (15Z)), C16 Ceramide (d14:1/16:0), C22 Ceramide (d14:1/22:0), C24:1 Ceramide (d18:2 (4E,8Z)/24:1 (15Z)), C16:0 Ceramide (d18:2 (4E,8Z)/16:0), C24:0 Ceramide (d18:2 (4E,8Z)/24:0), Ceramide (Egg), Ceramide (Brain), Brain CPE, CER1 (d18:1/26:0/18:1), CER9 (t18:0/26:0/18:1), Monomethyl Sphingosine (d18:1), Dimethyl Sphingosine (d18:1), Trimethyl Sphingosine (d18:1), Dimethyl Sphingosine-1-Phosphate (d18:1), Sphingosine-1-Phosphate (d18:1), Sphinganine-1-Phosphate (d18:0), Sphingosine-1-Phosphate (d17:1), Sphingosine-1-Phosphate (d17:1), Sphingosine-1-Phosphate (d20:1), Sphinganine-1-Phosphate (d20:0), N-12:0-1-deoxysphinganine, Sphinganine (d18:0), sphinganine (d17:0), Sphinganine (d20:0), 3-keto sphinganine (d18:0), 3-deoxy sphingosine, L-threo-sphingosine (d18:1), sphingosine (d18:1), Sphingosine (d17:1), Sphingosine (d20:1), Sphingosine (d22:1), 4E,14Z-Sphingadiene, 4E,8Z-Sphingadiene, 4E,11Z-Sphingadiene, Sphingosine (d16:1), Sphingosine (d14:1), Mito-So, and Brain Sphingosine.

In embodiments, the sphingolipid is of Formula (I):

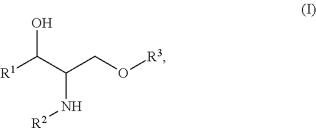

or a pharmaceutically acceptable salt thereof, wherein:
R¹ is a saturated aliphatic chain or an unsaturated aliphatic chain;
R² is H, or R⁴, or C(O)R⁴;
R³ is H, a sugar, P(O)(OH)₂, or P(O)(OH)O(CH₂)ₙR⁵ where n=1-10;
R⁴ is a saturated aliphatic chain or an unsaturated aliphatic chain; and
R⁵ is H, NH₂, N(CH₃)₃⁺, OH, or a sugar.

In embodiments, the sphingolipid is of Formula (I):

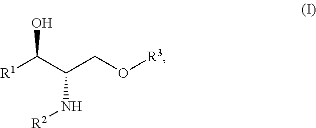

or a pharmaceutically acceptable salt thereof, wherein:
R¹ is a saturated aliphatic chain or an unsaturated aliphatic chain;
R² is H, or R⁴, or C(O)R⁴;
R³ is H, a sugar, P(O)(OH)₂, or P(O)(OH)O(CH₂)ₙR⁵ where n=1-10;
R⁴ is a saturated aliphatic chain or an unsaturated aliphatic chain; and
R⁵ is H, NH₂, N(CH₃)₃⁺, OH, or a sugar.

In embodiments of the sphingolipid of Formula (I), R¹ is an aliphatic chain.

In embodiments of the sphingolipid of Formula (I), R¹ is a C₄₋₃₀ aliphatic chain

In embodiments of the sphingolipid of Formula (I), R¹ is a saturated aliphatic chain.

In embodiments of the sphingolipid of Formula (I), R¹ is a C₄₋₃₀ saturated aliphatic chain.

In embodiments of the sphingolipid of Formula (I), R¹ is a C₄₋₃₀ alkyl or C₄₋₃₀ alkenyl.

In embodiments of the sphingolipid of Formula (I), R¹ is a C₄₋₃₀ alkenyl.

In embodiments of the sphingolipid of Formula (I), R¹ is a saturated C₁₅₋₁₇ aliphatic chain or a unsaturated C₁₅₋₁₇ aliphatic chain.

In embodiments of the sphingolipid of Formula (I), R¹ is a C₁₅₋₁₇ alkyl or a C₁₅₋₁₇ alkenyl.

In embodiments of the sphingolipid of Formula (I), R¹ is a saturated C₁₅₋₁₇ saturated aliphatic chain.

In embodiments of the sphingolipid of Formula (I), R¹ is a unsaturated C₁₅₋₁₇ aliphatic chain.

In embodiments of the sphingolipid of Formula (I), R¹ is a C₁₅₋₁₇ alkenyl.

In embodiments of the sphingolipid of Formula (I), R¹ is a saturated C₁₅ saturated aliphatic chain.

In embodiments of the sphingolipid of Formula (I), R¹ is a C₁₅ alkyl.

In embodiments of the sphingolipid of Formula (I), R¹ is a unsaturated C₁₅ aliphatic chain. In embodiments of the sphingolipid of Formula (I), R¹ is a C₁₅ alkenyl.

In embodiments of the sphingolipid of Formula (I), R¹ is a saturated C₁₇ saturated aliphatic chain.

In embodiments of the sphingolipid of Formula (I), R¹ is a C₁₇ alkyl.

In embodiments of the sphingolipid of Formula (I), R¹ is an unsaturated C₁₇ aliphatic chain.

In embodiments of the sphingolipid of Formula (I), R¹ is a C₁₇ alkenyl.

In embodiments, the sphingolipid of Formula (I) is of Formula (IV-A):

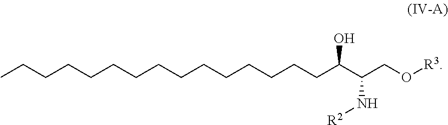

In embodiments, the sphingolipid of Formula (I) is of Formula (IV-B):

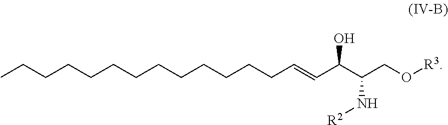

In embodiments of the sphingolipid of Formula (I), (IV-A), or (IV-B), R² is H, R⁴, or C(O)R⁴. In embodiments, R² is H. In embodiments, R² is R⁴. In embodiments, R² is C(O)R⁴.

In embodiments of the sphingolipid of Formula (I), (IV-A), or (IV-B), R³ is H, a sugar, P(O)(OH)₂, or P(O)(OH)O(CH₂)ₙR⁵ where n=1-10.

In embodiments of the sphingolipid of Formula (I), (IV-A), or (IV-B), R³ is —H, —P(O)(OH)₂ or a sugar.

In embodiments of the sphingolipid of Formula (I), (IV-A), or (IV-B), R³ is —H.

In embodiments of the sphingolipid of Formula (I), (IV-A), or (IV-B), R³ is —P(O)(OH)₂.

In embodiments of the sphingolipid of Formula (I), (IV-A), or (IV-B), R³ is P(O)(OH)O(CH₂)ₙR⁵ where n=1-10. In embodiments, n is 1, 2, 3, 4, 5, 6, 7, 8, 9, or 10. In embodiments, n is 1. In embodiments, n is 2. In embodiments, n is 3. In embodiments, n is 4. In embodiments, n is 5. In embodiments, n is 6. In embodiments, n is 7. In embodiments, n is 8. In embodiments, n is 9. In embodiments, n is 10.

In embodiments of the sphingolipid of Formula (I), (IV-A), or (IV-B), R³ is a sugar.

In embodiments, R³ is

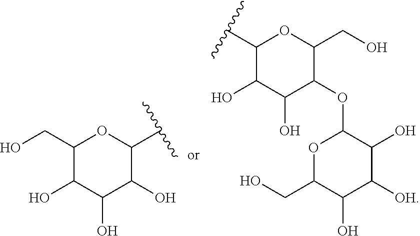 [SMILES: *C1OC(CO)C(O)C(O)C1O ; *C1OC(CO)C(OC2OC(CO)C(O)C(O)C2O)C(O)C1O]

In embodiments, R³ is

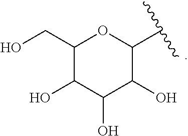 [SMILES: *C1OC(CO)C(O)C(O)C1O]

In embodiments, R³

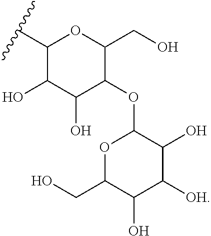 [SMILES: *C1OC(CO)C(OC2OC(CO)C(O)C(O)C2O)C(O)C1O]

In embodiments, R³ is

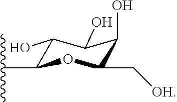

In embodiments, R³ is

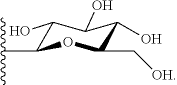

In embodiments, R³ is

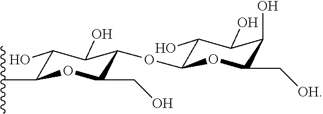

In embodiments of the sphingolipid of Formula (I), (IV-A), or (IV-B), R⁵ is H, NH₂, N(CH₃)₃⁺, OH, or a sugar.

In embodiments of the sphingolipid of Formula (I), (IV-A), or (IV-B), R⁵ is H, NH₂, OH, or a sugar.

In embodiments of the sphingolipid of Formula (I), (IV-A), or (IV-B), R⁵ is H.

In embodiments, R⁵ is NH₂.

In embodiments, R⁵ is N(CH₃)₃⁺.

In embodiments, R⁵ is OH.

In embodiments, R⁵ is a sugar.

In embodiments of the sphingolipid of Formula (I), (IV-A), or (IV-B), the sugar is:

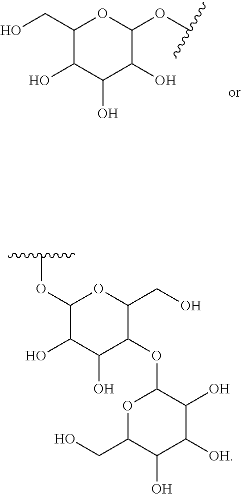 [SMILES: *OC1OC(CO)C(O)C(O)C1O ; *OC1OC(CO)C(OC2OC(CO)C(O)C(O)C2O)C(O)C1O]

In embodiments of the sphingolipid of Formula (I), (IV-A), or (IV-B), the sugar is

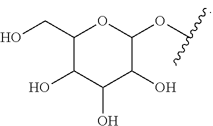 [SMILES: *OC1OC(CO)C(O)C(O)C1O]

In embodiments, the sugar is:

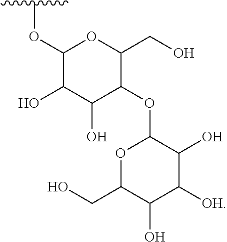 [SMILES: *OC1OC(CO)C(OC2OC(CO)C(O)C(O)C2O)C(O)C1O]

In embodiments of the sphingolipid of Formula (I), (IV-A), or (IV-B), the sugar is:

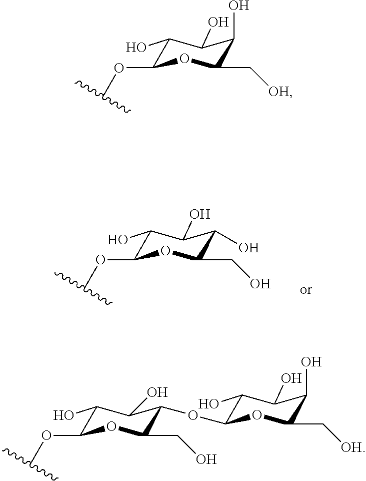

In embodiments of the sphingolipid of Formula (I), (IV-A), or (IV-B), the sugar is

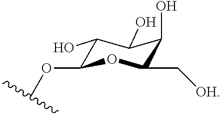

In embodiments, the sugar is

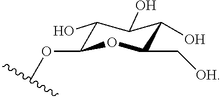

In embodiments, the sugar is:

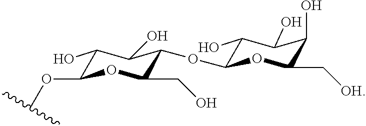

In embodiments of the sphingolipid of Formula (I), (IV-A), or (IV-B), R⁴ is a saturated aliphatic chain or an unsaturated aliphatic chain.

In embodiments of the sphingolipid of Formula (I), (IV-A), or (IV-B), R⁴ is a C₄₋₃₀ saturated aliphatic chain or a C₄₋₃₀ unsaturated aliphatic chain.

In embodiments of the sphingolipid of Formula (I), (IV-A), or (IV-B), R⁴ is a C₄₋₃₀ saturated aliphatic chain or a C₄₋₃₀ alkenyl.

In embodiments of the sphingolipid of Formula (I), (IV-A), or (IV-B), R⁴ is a C₄₋₃₀ alkyl or a C₄₋₃₀ alkenyl.

In embodiments of the sphingolipid of Formula (I), (IV-A), or (IV-B), R⁴ is a C₁₁₋₃₀ unsaturated aliphatic chain.

In embodiments of the sphingolipid of Formula (I), (IV-A), or (IV-B), R⁴ is a C₁₁₋₃₀ alkenyl.

In embodiments of the sphingolipid of Formula (I), (IV-A), or (IV-B), R⁴ is a C₁₁₋₃₀ saturated aliphatic chain.

In embodiments of the sphingolipid of Formula (I), (IV-A), or (IV-B), R⁴ is a C₁₁₋₃₀ alkyl.

In embodiments of the sphingolipid of Formula (I), (IV-A), or (IV-B), R⁴ is a C₁₁ unsaturated aliphatic chain.

In embodiments of the sphingolipid of Formula (I), (IV-A), or (IV-B), R⁴ is a C₁₁alkenyl.

In embodiments of the sphingolipid of Formula (I), (IV-A), or (IV-B), R⁴ is a C₁₅ unsaturated aliphatic chain.

In embodiments of the sphingolipid of Formula (I), (IV-A), or (IV-B), R⁴ is a C₁₅alkenyl.

In embodiments of the sphingolipid of Formula (I), (IV-A), or (IV-B), R⁴ is a C₁₇ unsaturated aliphatic chain.

In embodiments of the sphingolipid of Formula (I), (IV-A), or (IV-B), R⁴ is a C₁₇alkenyl.

In embodiments of the sphingolipid of Formula (I), (IV-A), or (IV-B), R⁴ is a C₂₃ unsaturated aliphatic chain.

In embodiments of the sphingolipid of Formula (I), (IV-A), or (IV-B), R⁴ is a C₂₃alkenyl.

In embodiments of the sphingolipid of Formula (I), (IV-A), or (IV-B), R⁴ is a C₁₁ saturated aliphatic chain.

In embodiments of the sphingolipid of Formula (I), (IV-A), or (IV-B), R⁴ is a C₁₁alkyl.

In embodiments of the sphingolipid of Formula (I), (IV-A), or (IV-B), R⁴ is a C₁₅ saturated aliphatic chain.

In embodiments of the sphingolipid of Formula (I), (IV-A), or (IV-B), R⁴ is a C₁₅alkyl.

In embodiments of the sphingolipid of Formula (I), (II), (III), (IV-A), or (IV-B), R⁴ is a C₁₇ saturated aliphatic chain.

In embodiments of the sphingolipid of Formula (I), (IV-A), or (IV-B), R⁴ is a C₁₇alkyl.

In embodiments of the sphingolipid of Formula (I), (IV-A), or (IV-B), R⁴ is a C₂₃ saturated aliphatic chain.

In embodiments of the sphingolipid of Formula (I), (IV-A), or (IV-B), R⁴ is a C₂₃alkyl.

In embodiments of the sphingolipid of Formula (I), (IV-A), or (IV-B), R⁴ is

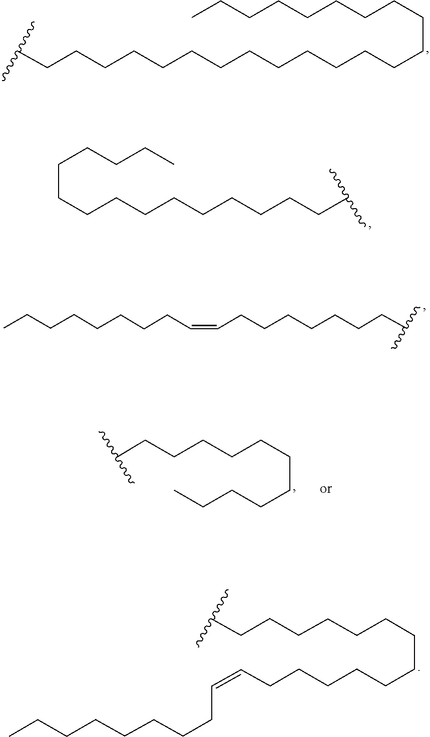 [SMILES: *CCCCCCCCCCCCCCCCCCCCCCC ; *CCCCCCCCCCCCCCC ; *CCCCCCC/C=C\CCCCCCCC ; *CCCCCCCCCCC ; *CCCCCCCCCCCCC/C=C\CCCCCCCC]

In embodiments of the sphingolipid of Formula (I), (IV-A), or (IV-B), R⁴ is

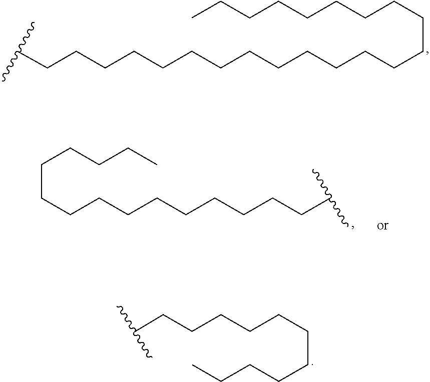 [SMILES: *CCCCCCCCCCCCCCCCCCCCCCC ; *CCCCCCCCCCCCCCC ; *CCCCCCCCCCC]

In embodiments of the sphingolipid of Formula (I), (IV-A), or (IV-B), R⁴ is

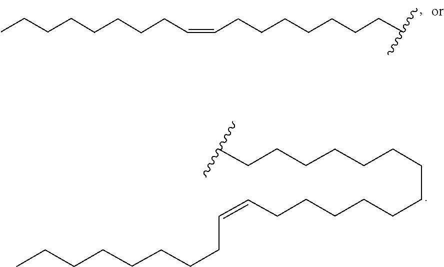 [SMILES: *CCCCCCC/C=C\CCCCCCCC ; *CCCCCCCCCCCCC/C=C\CCCCCCCC]

In embodiments of the sphingolipid of Formula (I), (IV-A), or (IV-B), R⁴ is

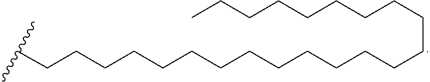 [SMILES: *CCCCCCCCCCCCCCCCCCCCCCC]

In embodiments of the sphingolipid of Formula (I), (IV-A), or (IV-B), R⁴ is

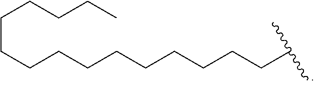 [SMILES: *CCCCCCCCCCCCCCC]

In embodiments of the sphingolipid of Formula (I), (IV-A), or (IV-B), R⁴ is

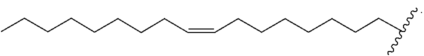 [SMILES: *CCCCCCC/C=C\CCCCCCCC]

In embodiments of the sphingolipids of Formula (I), (IV-A), or (IV-B), R⁴ is

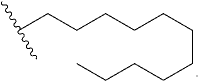 [SMILES: *CCCCCCCCCCC]

In embodiments of the sphingolipid of Formula (I), (IV-A), or (IV-B), R⁴ is

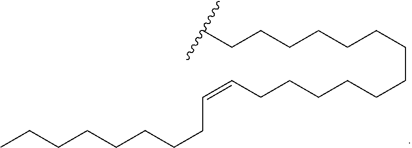 [SMILES: *CCCCCCCCCCCCC/C=C\CCCCCCCC]

In embodiments of the sphingolipid of Formula (I), (IV-A), or (IV-B), at least one of R¹ and R⁴ is unsaturated.

In embodiments of the sphingolipid of Formula (I),
R¹ is an unsaturated C₁₄₋₂₄ aliphatic chain;
R² is C(O)R⁴;
R³ is a sugar or H; and
R⁴ is a C₁₄₋₂₄ saturated aliphatic chain or a C₁₄₋₂₄ unsaturated aliphatic chain.

In embodiments of the sphingolipid of Formula (I),
R¹ is an unsaturated C₁₄₋₂₄ aliphatic chain;
R² is C(O)R⁴;
R³ is a sugar; and
R⁴ is a C₁₄₋₂₄ saturated aliphatic chain or a C₁₄₋₂₄ unsaturated aliphatic chain.

In embodiments of the sphingolipid of Formula (I),
R¹ is an unsaturated C₁₄₋₁₆ aliphatic chain (e.g., C₁₅ alkenyl);
R² is C(O)R⁴;
R³ is a sugar; and
R⁴ is a C₁₇₋₂₃ saturated aliphatic chain or a C₁₇₋₂₃ unsaturated aliphatic chain (e.g., C₁₇ alkenyl, C23 alkyl or a C23 alkenyl).

In embodiments of the sphingolipid of Formula (I),
R¹ is an unsaturated C₁₄₋₂₄ aliphatic chain;
R² is C(O)R⁴;
R³ is a sugar; and
R⁴ is a C₁₄₋₂₄ unsaturated aliphatic chain.

In embodiments of the sphingolipid of Formula (I),
R¹ is an unsaturated C₁₄₋₁₆ aliphatic chain (e.g., C₁₅ alkenyl);
R² is C(O)R⁴;
R³ is a sugar; and
R⁴ is a C₁₇₋₂₃ unsaturated aliphatic chain (e.g., C₁₇ alkenyl, C₂₃ alkyl or a C₂₃ alkenyl). In embodiments, the sphingolipid of Formula (I) is a sphingolipid of Formula (II):

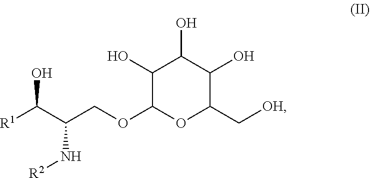

wherein
R¹ is an unsaturated C₁₄₋₂₄ aliphatic chain;
R² is C(O)R⁴; and
R⁴ is a C₁₄₋₂₄ unsaturated aliphatic chain.

In embodiments, the sphingolipid of Formula (II) is a sphingolipid of Formula (II-A) or (II-B):

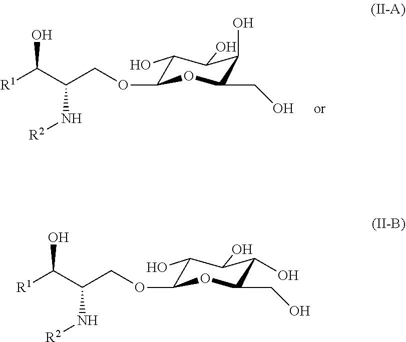

wherein
R¹ is an unsaturated C₁₄₋₂₄ aliphatic chain;
R² is C(O)R⁴; and
R⁴ is a C₁₄₋₂₄ unsaturated aliphatic chain.

In embodiments, the sphingolipid of Formula (II) is a sphingolipid of Formula (II-A):

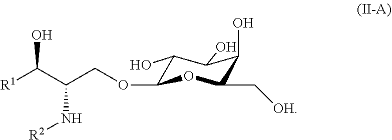

In embodiments, the sphingolipid of Formula (II) is a sphingolipid of Formula (II-B):

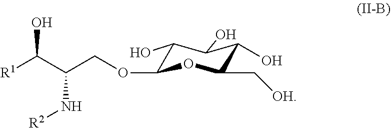

In embodiments of the compounds of Formula (II), (II-A), or (II-B), R¹ is a unsaturated C₁₅₋₁₇ aliphatic chain. In embodiment, R¹ is a C₁₅₋₁₇ alkenyl. In embodiments, R¹ is a C₁₅ alkenyl.

In embodiments of the compounds of Formula (II), (II-A), or (II-B), R⁴ is a C₁₇₋₂₃ unsaturated aliphatic chain. In embodiments, R⁴ is a C₁₇ unsaturated aliphatic chain, or a C₂₃ unsaturated aliphatic chain. In embodiments, R⁴ is a C₁₇₋₂₃ alkenyl. In embodiments, R⁴ is a C₁₇ alkenyl or a C₂₃ alkenyl.

In embodiments of the nanobiologic compositions provided herein, the sphingolipid is one or more sphingolipids selected from Table 1.

In embodiments of the nanobiologic compositions provided herein, the sphingolipid is one or more sphingolipids selected from Table 1, or a pharmaceutically acceptable salt thereof.

TABLE 1

| Entry | Sphingolipid |
|---|---|
| 1 | 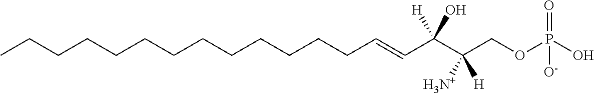 [SMILES: CCCCCCCCCCCCC/C=C/[C@@H](O)[C@@H]([NH3+])COP(=O)([O-])O] |
| | Sphingosine-1-phosphate (d18:1/16:0) |
| 2 | 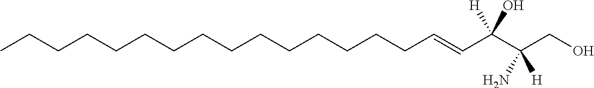 [SMILES: CCCCCCCCCCCCCCC/C=C/[C@@H](O)[C@@H](N)CO] |
| | Sphingosine (d20:1) |
| 3 | 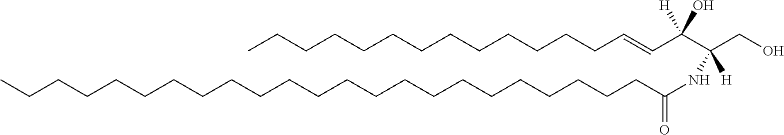 [SMILES: CCCCCCCCCCCCC/C=C/[C@@H](O)[C@H](CO)NC(=O)CCCCCCCCCCCCCCCCCCCCCCC] |
| | C24 Ceramide (d18:1/24:0) |
| 4 | 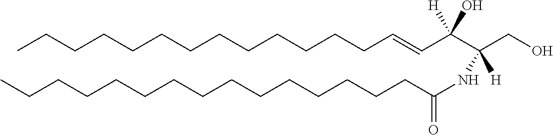 [SMILES: CCCCCCCCCCCCC/C=C/[C@@H](O)[C@H](CO)NC(=O)CCCCCCCCCCCCCCC] |
| | C16 Ceramide (d18:1/16:0) |
| 5 | 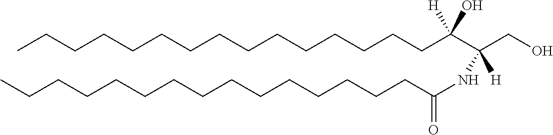 [SMILES: CCCCCCCCCCCCCCCC(=O)N[C@@H](CO)[C@H](O)CCCCCCCCCCCCCCC] |
| | C16 Dihydroceramide (18:0/16:0) |
| 6 | 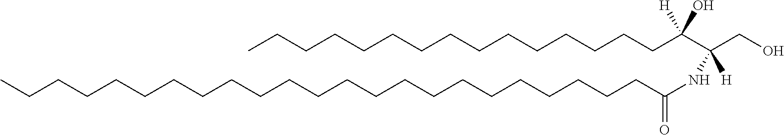 [SMILES: CCCCCCCCCCCCCCCCCCCCCCCC(=O)N[C@@H](CO)[C@H](O)CCCCCCCCCCCCCCC] |
| | C24 Dihydroceramide (d18:0/24:0) |
| 7 | 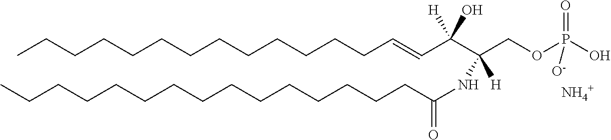 [SMILES: CCCCCCCCCCCCC/C=C/[C@@H](O)[C@H](COP(=O)([O-])O)NC(=O)CCCCCCCCCCCCCCC ; [NH4+]] |
| | C16 Ceramide-1-phosphate (d18:1/16:0) |
| 8 | 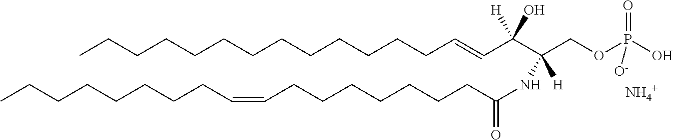 [SMILES: CCCCCCCC/C=C\CCCCCCCC(=O)N[C@@H](COP(=O)([O-])O)[C@H](O)/C=C/CCCCCCCCCCCCC ; [NH4+]] |
| | C18:1 Ceramide-1-phosphate (d18:1/18:1(9Z) |
| 9 | 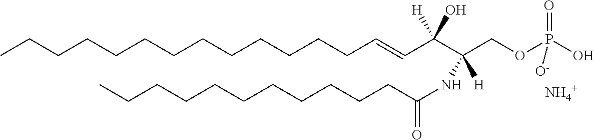 [SMILES: CCCCCCCCCCCCC/C=C/[C@@H](O)[C@H](COP(=O)([O-])O)NC(=O)CCCCCCCCCCC ; [NH4+]] |
| | C12 Ceramide-1-phosphate (d18:1/12:0) |
| 10 | 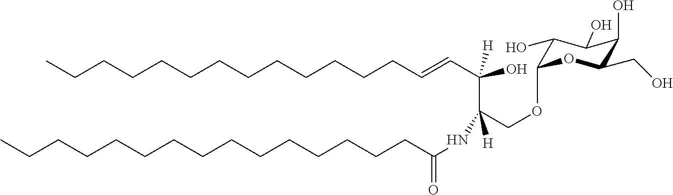 |
| | C16 Galactosyl(α) Ceramide (d18:1/16:0) |
| 11 | 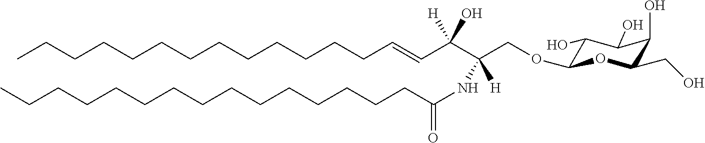 |
| | C16 Galactosyl(β) Ceramide (d18:1/16:0) |
| 12 | 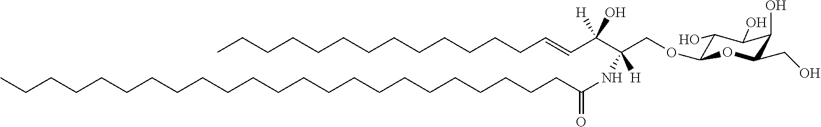 |
| | C24:0 Galactosyl(β) Ceramide (d18:1/24:0) |
| 13 | 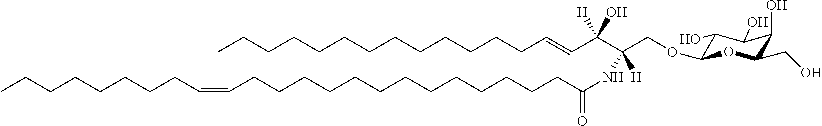 |
| | C24:1 Galactosyl(β) Ceramide (d18:1/24:1) |
| 14 | 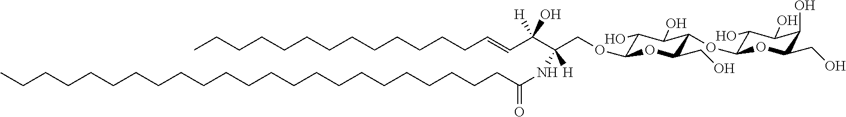 |
| | C24:0 Lactosyl(β) Ceramide (d18:1/24:0) |
| 15 | 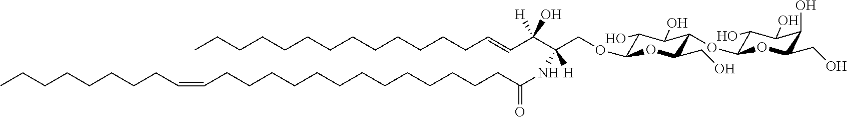 |
| | C24:1 Lactosyl(β) Ceramide (d18:1/24:1) |
| 16 | 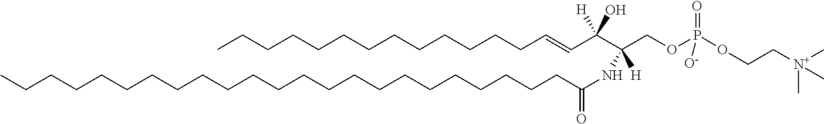 [SMILES: CCCCCCCCCCCCC/C=C/[C@@H](O)[C@H](COP(=O)([O-])OCC[N+](C)(C)C)NC(=O)CCCCCCCCCCCCCCCCCCCCCCC] |
| | 24:0 Sphingomyelin |
| 17 | 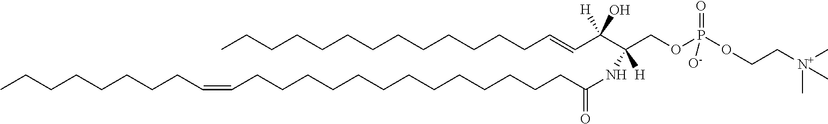 [SMILES: CCCCCCCC/C=C\CCCCCCCCCCCCCC(=O)N[C@@H](COP(=O)([O-])OCC[N+](C)(C)C)[C@H](O)/C=C/CCCCCCCCCCCCC] |
| | 24:1 Sphingomyelin |
| 18 | 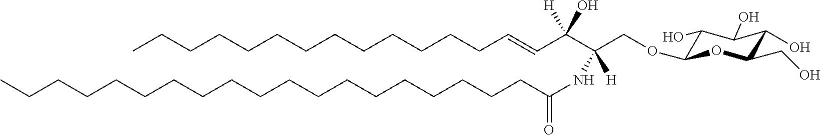 |
| | C18:0 Glucosyl(β) Ceramide (d18:1/18:0) |
| 19 | 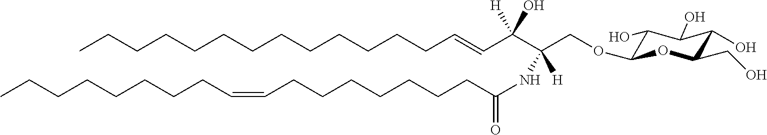 |
| | C18:1 Glucosyl(β) Ceramide (d18:1/18:1(9Z) |

In embodiments, the sphingolipid is selected from the group consisting of:

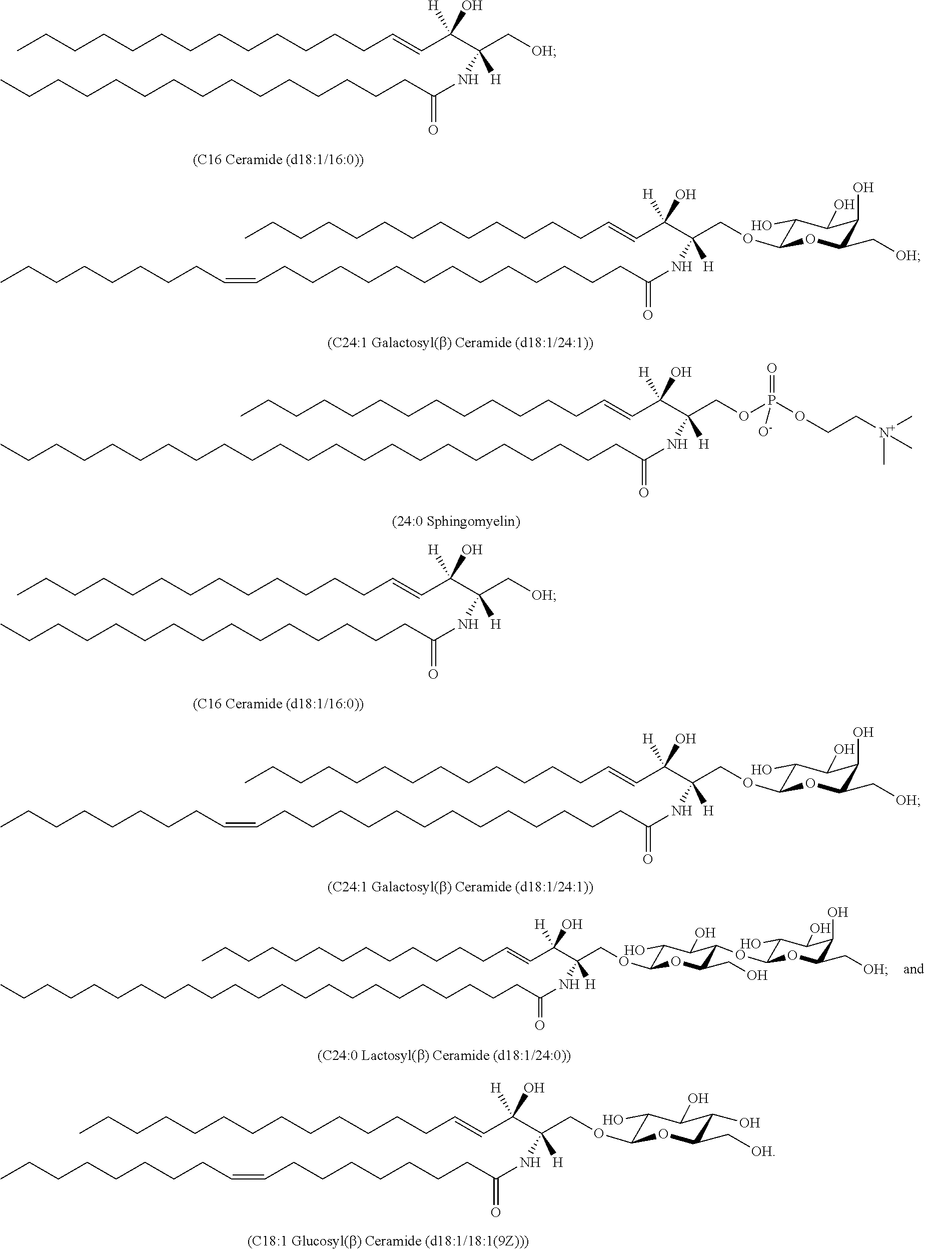

In embodiments, the sphingolipid is selected from the group consisting of:

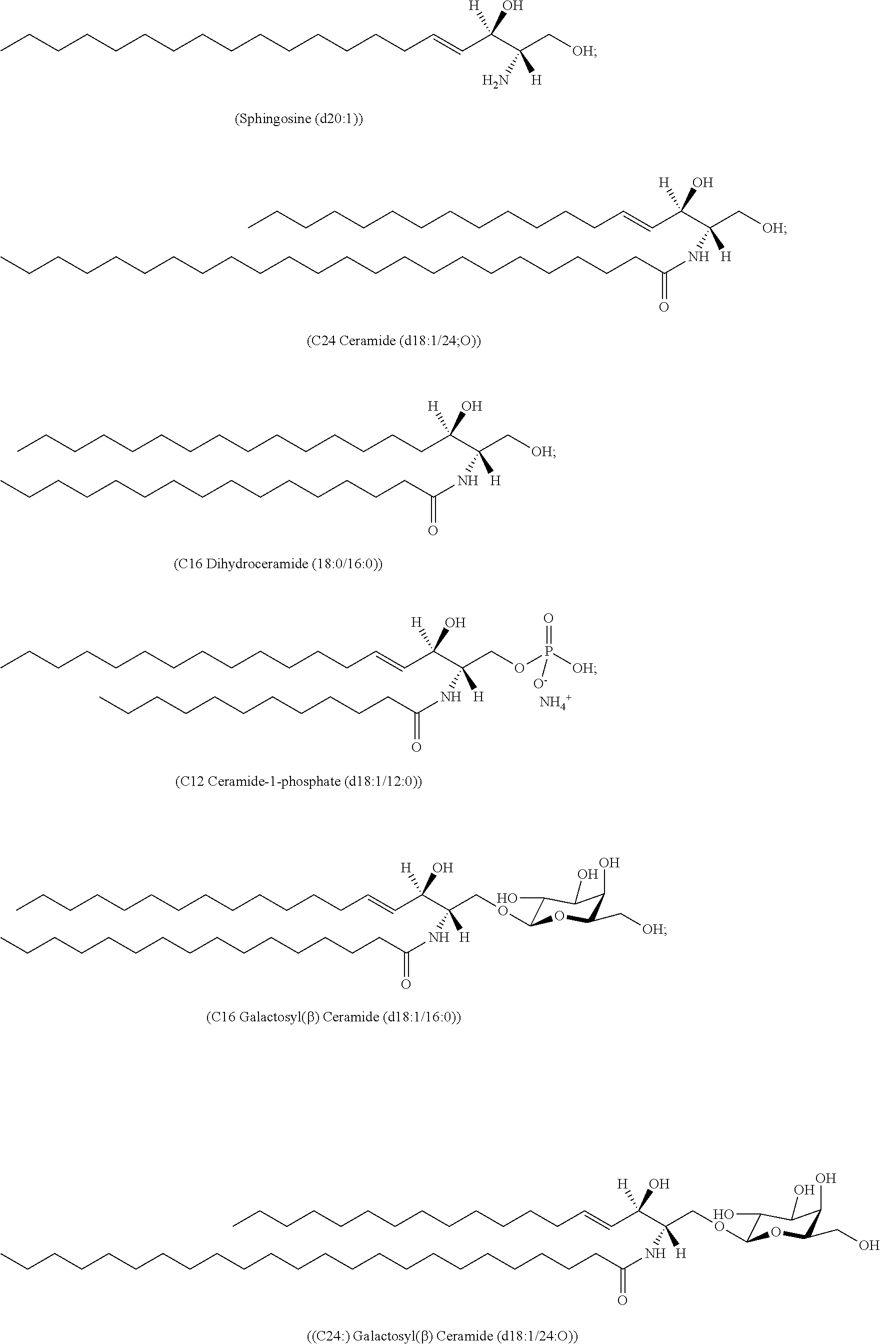

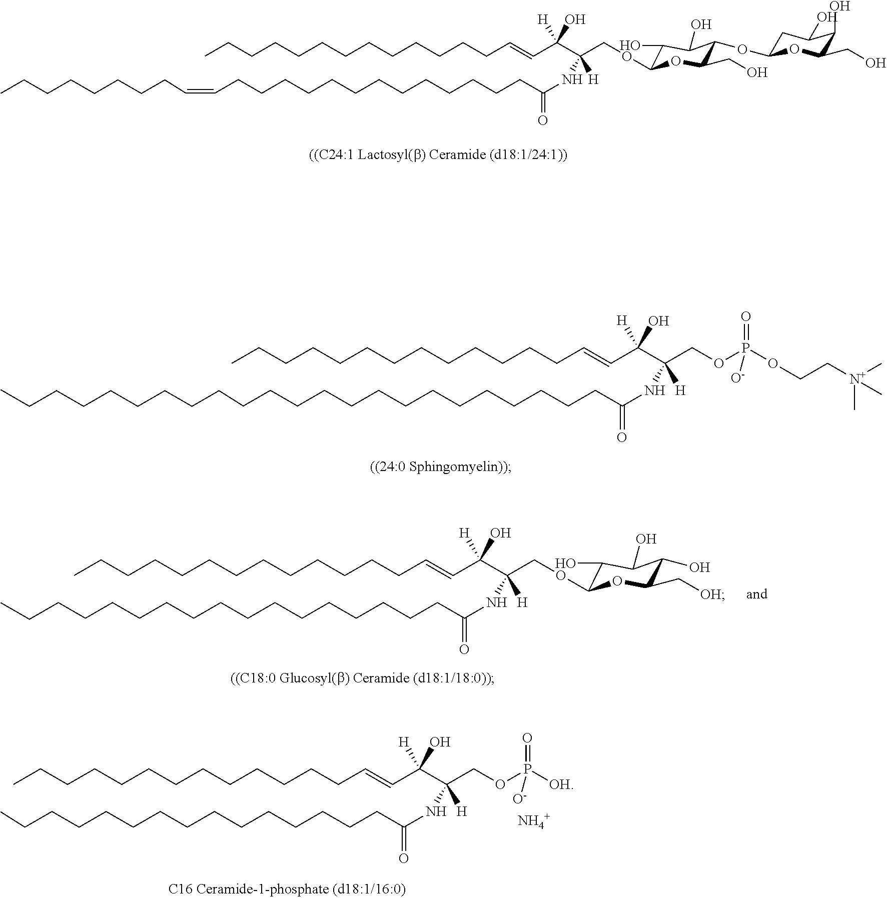

In embodiments of the nanobiologic compositions provided herein, the sphingolipid is:

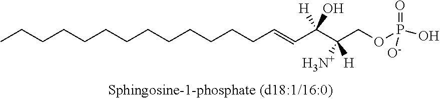 [SMILES: CCCCCCCCCCCCC/C=C/[C@@H](O)[C@@H]([NH3+])COP(=O)([O-])O]

In embodiments of the nanobiologic compositions provided herein, the sphingolipid is:

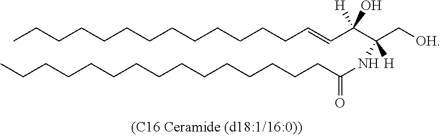 [SMILES: CCCCCCCCCCCCC/C=C/[C@@H](O)[C@H](CO)NC(=O)CCCCCCCCCCCCCCC]

In embodiments of the nanobiologic compositions provided herein, the sphingolipid is:

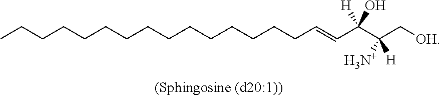 [SMILES: CCCCCCCCCCCCCCC/C=C/[C@@H](O)[C@@H]([NH3+])CO]

In embodiments of the nanobiologic compositions provided herein, the sphingolipid is:

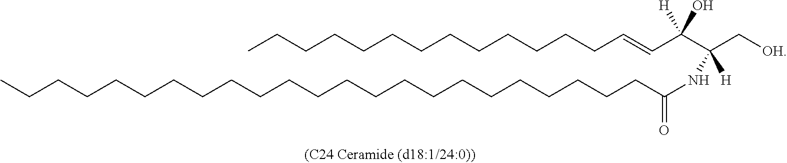 [SMILES: CCCCCCCCCCCCC/C=C/[C@@H](O)[C@H](CO)NC(=O)CCCCCCCCCCCCCCCCCCCCCCC]

In embodiments of the nanobiologic compositions provided herein, the sphingolipid is

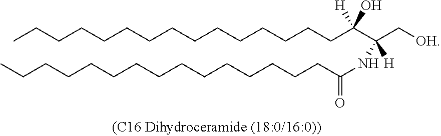 [SMILES: CCCCCCCCCCCCCCCC(=O)N[C@@H](CO)[C@H](O)CCCCCCCCCCCCCCC]

In embodiments of the nanobiologic compositions provided herein, the sphingolipid is

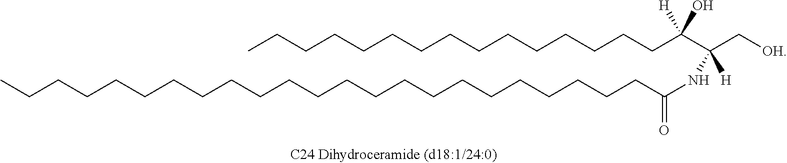 [SMILES: CCCCCCCCCCCCCCCCCCCCCCCC(=O)N[C@@H](CO)[C@H](O)CCCCCCCCCCCCCCC]

In embodiments of the nanobiologic compositions provided herein, the sphingolipid is

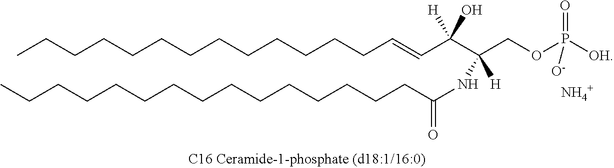 [SMILES: CCCCCCCCCCCCC/C=C/[C@@H](O)[C@H](COP(=O)([O-])O)NC(=O)CCCCCCCCCCCCCCC ; [NH4+]]

In embodiments of the nanobiologic compositions provided herein, the sphingolipid is

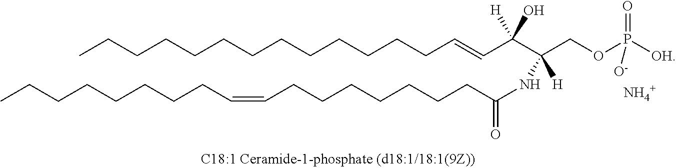 [SMILES: CCCCCCCC/C=C\CCCCCCCC(=O)N[C@@H](COP(=O)([O-])O)[C@H](O)/C=C/CCCCCCCCCCCCC ; [NH4+]]

In embodiments of the nanobiologic compositions provided herein, the sphingolipid is

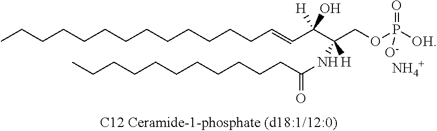 [SMILES: CCCCCCCCCCCCC/C=C/[C@@H](O)[C@H](COP(=O)([O-])O)NC(=O)CCCCCCCCCCC ; [NH4+]]

In embodiments of the nanobiologic compositions provided herein, the sphingolipid is

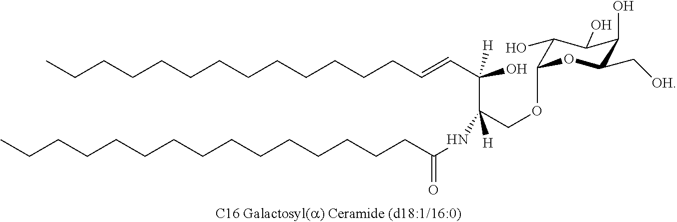

In embodiments of the nanobiologic compositions provided herein, the sphingolipid is

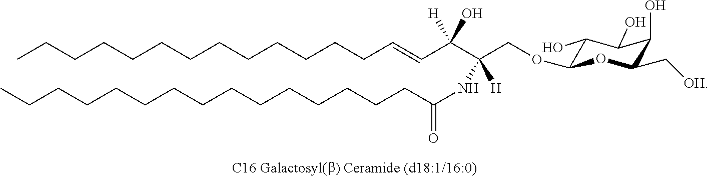

In embodiments of the nanobiologic compositions provided herein, the sphingolipid is

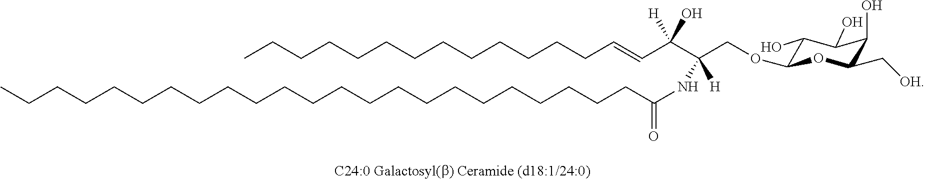

In embodiments of the nanobiologic compositions provided herein, the sphingolipid is

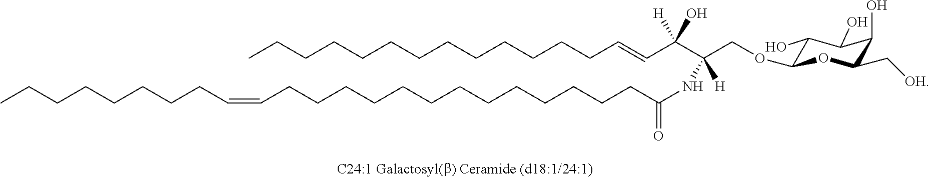

In embodiments of the nanobiologic compositions provided herein, the sphingolipid is

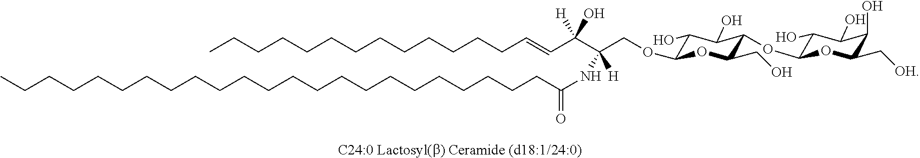

In embodiments of the nanobiologic compositions provided herein, the sphingolipid is

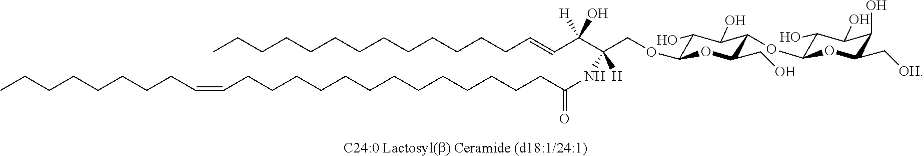

In embodiments of the nanobiologic compositions provided herein, the sphingolipid is

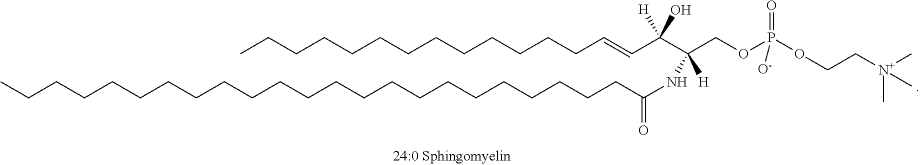 [SMILES: CCCCCCCCCCCCC/C=C/[C@@H](O)[C@H](COP(=O)([O-])OCC[N+](C)(C)C)NC(=O)CCCCCCCCCCCCCCCCCCCCCCC]

In embodiments of the nanobiologic compositions provided herein, the sphingolipid is

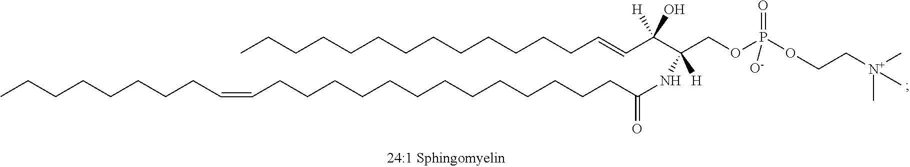 [SMILES: CCCCCCCC/C=C\CCCCCCCCCCCCCC(=O)N[C@@H](COP(=O)([O-])OCC[N+](C)(C)C)[C@H](O)/C=C/CCCCCCCCCCCCC]

In embodiments of the nanobiologic compositions provided herein, the sphingolipid is

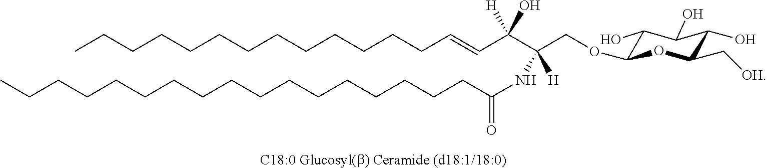

In embodiments of the nanobiologic compositions provided herein, the sphingolipid is

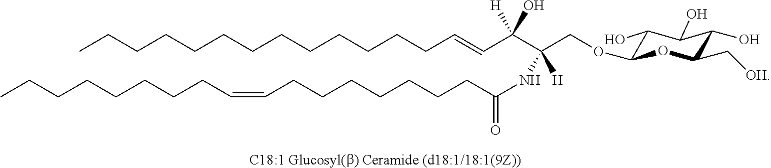

In embodiments of the nanobiologic compositions provided herein, the sphingolipid is present in about, or at least about, 1-100 mol % of the total lipid composition of the nanobiologic composition, including about or at least about 0.1 mol %, about or at least about 0.5 mol %, about or at least about 0.75 mol %, about or at least about 1% mol %, about or at least about 2 mol %, about or at least about 3 mol %, about, or at least about 4 mol %, about or at least about 5 mol %, about or at least about 6 mol %, about or at least about 7 mol %, about or at least about 8 mol %, about or at least about 9 mol %, about or at least about 10 mol %, about or at least about 11 mol %, about or at least about 12 mol %, about or at least about 13 mol %, about or at least about 14 mol %, about or at least about 15 mol %, about or at least about 16 mol %, about or at least about 17 mol %, about or at least about 18 mol %, about or at least about 19 mol %, about or at least about 20 mol %, about or at least about 21 mol %, about or at least about 22 mol %, about or at least about 23 mol %, about or at least about 24 mol %, about or at least about 25 mol %, about or at least about 26 mol %, about or at least about 27 mol %, about or at least about 28 mol %, about or at least about 29 mol %, about or at least about 30 mol %, about or at least about or at least about 35 mol %, about or at least about 40 mol %, about or at least about 45 mol %, about or at least about 50 mol %, about or at least about 55 mol %, about or at least about 60 mol %, about or at least about 65%, about or at least about 70%, about or at least about 75%, about or at least about 80%, about or at least about 85%, about or at least about 90%, about or at least about 95% to about 100%, including all values and subranges therebetween.

In embodiments of the nanobiologic compositions provided herein, the sphingolipid is present as about 10-25 mol % of the total lipid composition of the nanobiologic composition.

In embodiments of the nanobiologic compositions provided herein, the sphingolipid is present as about 20 mol % of the total lipid composition of the nanobiologic composition.

In embodiments, the nanobiologic composition comprises human apolipoprotein A-I (apoA-I); for example, the ApoA-I sequence may have the mature peptide sequence shown below, or may include either or both of a signal peptide (e.g., SEQ ID NO:347) or a pro-peptide portion (e.g., SEQ ID NO:348). The peptide may be manufactured by expressing a nucleic acid encoding all three of the signal peptide, pro-peptide portion and the mature peptide. During protein synthesis and maturation the mature peptide is released and can be purified from the host cell for use in the compositions herein. In certain host cells, such as bacteria, the encoded peptide may not be cleaved into a mature peptide. An N-terminal methionine (M) residue of the Apo-A1 peptide used herein may be a formyl-methionine (fM) residue. In embodiments, the ApoA-I used in compositions herein comprises or consists of the sequence of SEQ ID NO:349 or a sequence having 1, 2, 4, 5, 6, 7, 8, 9, 10, or up to 20 conservative substitutions thereto.

TABLE 2A
ApoA-I peptides

| Peptide | SEQ ID NO: | Sequence |
|---|---|---|
| Signal Peptide | 347 | MKAAVLTLAV LFLTGSQA |
| Pro-peptide | 348 | RH FWQQ |
| portion | | |
| Mature Peptide | 349 | DEPPQSPWDRVKDLATVYVDVLKDSGRDYVSQFEG |
| | | SALGKQLNLKLLDNWDSVTSTFSKLREQLGPVTQEF |
| | | WDNLEKETEGLRQEMSKDLEEVKAKVQPYLDDFQK |
| | | KWQEEMELYRQKVEPLRAELQEGARQKLHELQEKL |
| | | SPLGEEMRDRARAHVDALRTHLAPYSDELRQRLAAR |
| | | LEALKENGGARLAEYHAKATEHLSTLSEKAKPALED |
| | | LRQGLLPVLESFKVSFLSALEEYTKKLNTQ |

In embodiments, the nanobiologic composition comprises a peptide mimetic of apolipoprotein A-I (apoA-I). Suitable apoA-I mimetic polypeptides may have the sequence shown in Table 2B (SEQ ID NOS: 256 to 263, and 342 to 346) or in SEQ ID NOS: 1 to 341.

TABLE 2B
ApoA-I mimetics

| SEQ ID NO | Amino Acid Sequence |
|---|---|
| 256 | DWLKAFYDKVAEKLKEAF (18A) |
| 257 | Ac-DWLKAFYDKVAEKLKEAF-NH₂ (2F) |
| 260 | Ac-DWFKAFYDKVAEKFKEAF-NH₂ (4F) |
| 258 | Ac-DWFKAFYDKVAEKLKEAF-NH₂ (3^{F3}) |
| 259 | Ac-DWLKAFYDKVAEKFKEAF-NH₂ (3^{F14}) |
| 261 | Ac-DWLKAFYDKVFEKFKEFF-NH₂ (5F) |
| 262 | Ac-DWLKAFYDKFFEKFKEFF-NH₂ (6F |
| 263 | Ac-DWFKAFYDKFFEKFKEFF-NH₂ (7F) |
| 342 | Ac-FWLKAFYDKVAEKLKEAF-NH₂ (3F-1) |
| 343 | Ac-DFLKAFYDKVAEKLKEAF-NH₂ (3F-2) |
| 344 | Ac-DWFRAFYDKVAEKFREAF-NH₂ (4F-R)^{q} |
| 345 | Ac-DWFKAFYDRVAERFKEAF-NH₂ (4F-R′)^{q} |
| 346 | Ac-DWLXAFYDXVAEXLXEAF-NH₂ (2F′) |

In embodiments, the apoA-I mimetic is DWLKAFYDKVAEKLKEAF (SEQ ID NO. 256). In embodiments, the apoA-I mimetic is Ac-DWLKAFYDKVAEKLKEAF-NH₂ (SEQ ID NO. 257). In embodiments, the apoA-I mimetic is Ac-DWFKAFYDKVAEKFKEAF-NH₂ (SEQ ID NO. 260).

In embodiments, apoA-I mimetics are optionally acetylated on the N-terminus, or optionally amidated on the C-terminus. In embodiments, the apoA-I mimetics are acetylated on the N-terminus. In embodiments, the apoA-I mimetics are amidated on the C-terminus. In embodiments, the apoA-I mimetics are acetylated on the N-terminus and amidated on the C-terminus.

In embodiments, the nanobiologic compositions of the present disclosure comprise one or more phospholipids. Examples of suitable phospholipids include, without limitation, phosphatidylcholines, phosphatidylethanolamines, phosphatidylinositol, phosphatidylserines, as well as phospholipid-containing oils such as lecithin oils. Non-limiting examples of the phospholipids that may be used in the present composition include phosphatidylcholines (PC), phosphatidylglycerols (PG), phosphatidylserines (PS), phosphatidylethanolamines (PE), and phosphatidic acid/esters (PA).

In embodiments, the phospholipid is independently selected from the group consisting of a phosphatidylcholine, a phosphatidylethanolamine, a phosphatidylinositol, a phosphatidylserine, a phospholipid-containing oil, a phosphatidylglycerol, a phosphatidic acid, and combinations thereof.

In embodiments, the phospholipid is one or more of the following: DDPC CAS-3436-44-0 1,2-Didecanoyl-sn-glycero-3-phosphocholine, DEPA-NA CAS-80724-31-8 1,2-Dierucoy 1-sn-glycero-3-phosphate (Sodium Salt), DEPC CAS-56649-39-9 1,2-Dierucoyl-sn-glycero-3-phosphocholine, DEPE CAS-988-07-2 1,2-Dierucoyl-sn-glycero-3-phosphoethanolamine, DEPG-NA 1,2-Dierucoyl-sn-glycero-3-phospho-rac-(1-glycerol) (Sodium Salt), DLOPC CAS-998-06-1, 1,2-Dilinoleoyl-sn-glycero-3-phosphocholine, DLPA-NA 1,2-Dilauroyl-sn-glycero-3-phosphate (Sodium Salt), DLPC CAS-18194-25-7 1,2-Dilauroyl-sn-glycero-3-phosphocholine, DLPE 1,2-Dilauroyl-sn-glycero-3-phosphoethanolamine, DLPG-NA 1,2-Dilauroyl-sn-glycero-3-phospho-rac-(1-glycerol) (Sodium Salt), DLPG-NH4 1,2-Dilauroyl-sn-glycero-3-phospho-rac-(1-glycerol) (Ammonium Salt), DLPS-NA 1,2-Dilauroyl-sn-glycero-3-phosphoserine (Sodium Salt), DMPA-NA CAS-80724-3 1,2-Dimyristoyl-sn-glycero-3-phosphate (Sodium Salt), DMPC CAS-18194-24-6 1,2-Dimyristoyl-sn-glycero-3-phosphocholine, DMPE CAS-988-07-2 1,2-Dimyristoyl-sn-glycero-3-phosphoethanolamine, DMPG-NA CAS-67232-80-8 1,2-Dimyristoyl-sn-glycero-3-phospho-rac-(1-glycerol) (Sodium Salt), DMPG-NH4 1,2-Dimyristoyl-sn-glycero-3-phospho-rac-(1-glycerol) (Ammonium Salt), DMPG-NH4/NA 1,2-Dimyristoyl-sn-glycero-3-phospho-rac-(lglycerol) (Sodium/Ammonium Salt), DMPS-NA 1,2-Dimyristoyl-sn-glycero-3-phosphoserine (Sodium Salt), DOPA-NA 1,2-Dioleoyl-sn-glycero-3-phosphate (Sodium Salt), DOPC CAS-4235-95-4 1,2-Dioleoyl-sn-glycero-3-phosphocholine, DOPE CAS-4004-5-1 1,2-Dioleoyl-sn-glycero-3-phosphoethanolamine, DOPG-NA CAS-62700-69-0 1,2-Dioleoyl-sn-glycero-3-phospho-rac-(1-glycerol) (Sodium Salt), DOPS-NA CAS-70614-14-1, 1,2-Dioleoyl-sn-glycero-3-phosphoserine (Sodium Salt), DPPA-NA CAS-71065-87-7, 1,2-Dipalmitoyl-sn-glycero-3-phosphate (Sodium Salt), DPPC CAS-63-89-8, 1,2-Dipalmitoyl-sn-glycero-3-phosphocholine, DPPE CAS-923-61-5 1,2-Dipalmitoyl-sn-glycero-3-phosphoethanolamine, DPPG-NA CAS-67232-81-9 1,2-Dipalmitoyl-sn-glycero-3-phospho-rac-(1-glycerol) (Sodium Salt), DPPG-NH4 CAS-73548-70-6 1,2-Dipalmitoylsn-glycero-3-phospho-rac-(1-glycerol) (Ammonium Salt), DPPS-NA 1,2-Dipalmitoyl-sn-glycero-3-phosphoserine (Sodium Salt), DSPA-NA CAS-108321-18-2 1,2-Distearoyl-snglycero-3-phosphate (Sodium Salt), DSPC CAS-816-94-4 1,2-Distearoyl-sn-glycero-3-phosphocholine, DSPE CAS-1069-79-0 1,2-Distearoyl-sn-glycero-3-phosphoethanolamine, DSPG-NA CAS-67232-82-0 1,2-Distearoyl-sn-glycero-3-phospho-rac-(1-glycerol) (Sodium Salt), DSPG-NH4 CAS-108347-80-4 1,2-Distearoyl-sn-glycero-3-phospho-rac-(1-glycerol) (Ammonium Salt), DSPS-NA 1,2-Distearoyl-sn-glycero-3-phosphoserine (Sodium Salt), EPC Egg-PC, HEPC Hydrogenated Egg PC, HSPC Hydrogenated Soy PC, MPPC 1-Myristoyl-2-palmitoyl-sn-glycero 3-phosphocholine, MSPC 1-Myristoyl-2-stearoyl-sn-glycero-3-phosphocholine, PMPC 1-Palmitoyl-2-myristoyl-sn-glycero-3-phosphocholine, POPC CAS-26853-31-6 1-Palmitoyl-2-oleoyl-sn-glycero-3-phosphocholine, POPE 1-Palmitoyl-2-oleoyl-sn-glycero-3-phosphoethanolamine, POPG-NA CAS-81490 May 3 1-Palmitoyl-2-oleoyl-sn-glycero-3 [Phospho-rac-(1-glycerol)] (Sodium Salt), PSPC 1-Palmitoyl-2-stearoyl-sn-glycero-3-phosphocholine, SMPC 1-Stearoyl-2-myristoyl-snglycero-3-phosphocholine, SOPC 1-Stearoyl-2-oleoyl-sn-glycero-3-phosphocholine, SPPC1-Stearoyl 1-2-palmitoyl 1-sn-glycero-3-phosphocholine.

In embodiments, the phospholipid is dimyristoylphosphatidylcholine (DMPC), soy lecithin, dipalmitoylphosphatidylcholine (DPPC), distearoylphosphatidylcholine (DSPC), dilaurylolyphosphatidylcholine (DLPC), dioleoylphosphatidylcholine (DOPC), dilaurylolylphosphatidylglycerol (DLPG), dimyristoylphosphatidylglycerol (DMPG), dipalmitoylphosphatidylglycerol (DPPG), distearoylphosphatidylglycerol (DSPG), dioleoylphosphatidylglycerol (DOPG), dimyristoyl phosphatidic acid (DMPA), dimyristoyl phosphatidic acid (DMPA), dipalmitoyl phosphatidic acid (DPPA), dipalmitoyl phosphatidic acid (DPPA), dimyristoyl phosphatidylethanolamine (DMPE), dipalmitoyl phosphatidylethanolamine (DPPE), dimyristoyl phosphatidylserine (DMPS), dipalmitoyl phosphatidylserine (DPPS), and mixtures thereof.

In embodiments of the nanobiologic compositions provided herein, the phospholipid is selected from the group consisting of 1,2-dimyristoyl-sn-glycero-3-phosphocholine (DMPC), 1-palmitoyl-2-oleoyl-sn-glycero-3-phosphocholine (POPC), 1,2-dioleoyl-sn-glycero-3-phosphocholine (DOPC) and mixtures thereof.

In embodiments of the nanobiologic compositions provided herein, the phospholipid is 1,2-dimyristoyl-sn-glycero-3-phosphocholine (DMPC) or 1-palmitoyl-2-oleoyl-sn-glycero-3-phosphocholine (POPC).

In embodiments of the nanobiologic compositions provided herein, the phospholipid is DMPC.

In embodiments, the nanobiologic compositions comprises a phospholipid and a lysophospholipid.

In embodiments, the lysophospholipid is a lysophosphatidylcholine.

In embodiments, the lysophospholipid is LYSOPC MYRISTIC 1-Myristoyl-sn-glycero-3-phosphocholine, LYSOPC PALMITIC CAS-17364-16-8 1-Palmitoyl-sn-glycero-3-phosphocholine, or LYSOPC STEARIC CAS-19420-57-6 1-Stearoyl-sn-glycero-3-phosphocholine,

In embodiments of the nanobiologic compositions provided herein, the lysophospholipid is selected from the group consisting of 1-myristoyl-2-hydroxy-sn-glycero-3-phosphocholine (MHPC), 1-palmitoyl-2-hydroxy-sn-glycero-3-phosphocholine (PHPC), 1-stearoyl-2-hydroxy-sn-glycero-3-phosphocholine (SHPC), and mixtures thereof.

In embodiments of the nanobiologic compositions provided herein, the phospholipid is 1-palmitoyl-2-oleoyl-sn-glycero-3-phosphocholine (POPC) and the lysophospholipid is 1-palmitoyl-2-hydroxy-sn-glycero-3-phosphocholine (PHPC).

In embodiments, when the composition comprises two types of phospholipid, the weight ratio of two types of phospholipids ranges from about 1:10 to about 10:1, including about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, to about 10:1, including all values and ranges therebetween.

In embodiments, when the composition comprises a phospholipid and a lysophospholipid, the weight ratio of two phospholipid and lysophospholipid ranges from about 1:10 to about 10:1, including about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, to about 10:1, including all values and ranges therebetween.

In embodiments the nanobiologic compositions of the present disclosure comprise a sterol, such as cholesterol. Typically, the nanobiologic composition comprise from about 1 mol % to about 100 mol % of cholesterol relative to phospholipid, including about 1% mol %, about 2 mol %, about 3 mol %, about 4 mol %, about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, about 20 mol %, about 21 mol %, about 22 mol %, about 23 mol %, about 24 mol %, about 25 mol %, about 26 mol %, about 27 mol %, about 28 mol %, about 29 mol %, about 30 mol %, about 35 mol %, about 40 mol %, about 45 mol %, about 50 mol %, about 55 mol %, about 60 mol %, about 65 mol %, about 70 mol %, about 75 mol %, about 80 mol %, about 85 mol %, about 90 mol %, about 95 mol %, to about 100 mol % (i.e. 1:1 mol/mol mixture of cholesterol and phospholipid (e.g. DMPC)) including all ranges and values therebetween. In embodiments, the nanobiologic composition comprises from about 1 mol % to about 30 mol % cholesterol. In embodiments, the nanobiologic composition comprises from about 15 mol % to about 25 mol % cholesterol, relative to phospholipid. In embodiments, the nanobiologic composition comprises from about 20 mol % cholesterol, relative to phospholipid. In embodiments, the nanobiologic composition comprises from about 10 mol % to about 35 mol % cholesterol, relative to phospholipid. In embodiments, the nanobiologic composition comprises from about 15 mol % to about 30 mol % cholesterol, relative to phospholipid. In embodiments, the nanobiologic composition comprises from about 15 mol % to about 25 mol % cholesterol, relative to phospholipid. In embodiments, the nanobiologic composition comprises from about 28 mol % to about 23 mol % cholesterol, relative to phospholipid. In embodiments, the nanobiologic composition comprises from about 20 mol % to about 27 mol % cholesterol, relative to phospholipid.

Without being bound by theory, the addition of cholesterol may stabilize the nanobiologic composition and improve entrapment efficiency.

In embodiments of the nanobiologic compositions provided herein, the cholesterol is present in about 1-30 mol % relative to the phospholipid.

In embodiments of the nanobiologic compositions provided herein, the cholesterol is present in about 5-25 mol % relative to the phospholipid.

In embodiments of the nanobiologic compositions provided herein, the nanobiologic composition is cholesterol free.

In embodiments of the nanobiologic compositions provided herein, the molar ratio of cholesterol:phospholipid, in the nanobiologic composition is about 0:1, about 0.025:1, about 0.05:1, about 0.075:1, about 0.1:1, about 0.125:1, about 0.15:1, about 0.175:1, about 0.2:1, about 0.225:1, about 0.25:1, about 0.275:1, about 0.3:1, about 0.325:1, about 0.35:1, about 0.375:1, about 0.4:1, about 0.425:1, about 0.45:1, about 0.475:1 or about 0.5:1, including all values therebetween. In embodiments, the molar ratio of cholesterol:phospholipids ranges from about 0:1 to about 0.5:1, including about 0:1, about 0.025:1, about 0.05:1, about 0.075:1, about 0.1:1, about 0.125:1, about 0.15:1, about 0.175:1, about 0.2:1, about 0.225:1, about 0.25:1, about 0.275:1, about 0.3:1, about 0.325:1, about 0.35:1, about 0.375:1, about 0.4:1, about 0.425:1, about 0.45:1, about 0.475:1 to about 0.5:1, including all ranges therebetween. In embodiments, the molar ratio of cholesterol:phospholipids ranges from about 0.05:1 to about 0.25:1. In embodiments, the molar ratio of cholesterol is about 0.2:1.

In embodiments, the weight percentage of cholesterol ranges from about 0% (w/w) to about 15% (w/w) of the lipid, or nanobiologic composition, including from about 1% (w/w), about 1.5% (w/w), about 2% (w/w), about 2.5% (w/w), about 3% (w/w), about 3.5% (w/w), about 4% (w/w), about 4.5% (w/w), about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), about 10% (w/w), about 10.5% (w/w), about 11% (w/w/), about 11.5% (w/w), about 12% (w/w), about 12.5% (w/w), about 13% (w/w), about 13.5% (w/w), about 14% (w/w), about 14.5% (w/w), to about 15% (w/w). In embodiments, the weight percentage of cholesterol ranges from about 0% (w/w) to about 15%, (w/w) of the nanoparticle, lipid, or composition, including from about 1% (w/w), about 1.5% (w/w), about 2% (w/w), about 2.5% (w/w), about 3% (w/w), about 3.5% (w/w), about 4% (w/w), about 4.5% (w/w), about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), about 10% (w/w), about 10.5% (w/w), about 11% (w/w/), about 11.5% (w/w), about 12% (w/w), about 12.5% (w/w), about 13% (w/w), about 13.5% (w/w), about 14% (w/w), about 14.5% (w/w), to about 15% (w/w). In embodiments, the weight percentage is the weight percentage of cholesterol relative to phospholipids.). In embodiments, the weight percentage is the weight percentage of cholesterol relative to total lipids. In embodiments, the weight percentage is the weight percentage of cholesterol relative to the nanobiologic composition. In embodiments, the weight percentage of cholesterol ranges from about 1 to 10% cholesterol (w/w %) of the nanobiologic composition, the weight percentage of cholesterol ranges from about 2 to 8% cholesterol (w/w %) of the nanobiologic composition. In embodiments, the weight percentage of cholesterol ranges from about 3.5 to 7.5% cholesterol (w/w %) of the nanobiologic composition. In embodiments, the weight percentage of cholesterol ranges from about 5 to 10% cholesterol (w/w %) of the nanobiologic composition. In embodiments, the weight percentage of cholesterol is about 3.6 (w/w %) of the nanobiologic composition. In embodiments, the weight percentage of cholesterol is about 7.2 (w/w %) of the nanobiologic composition. In embodiments, the weight percentage of cholesterol is about 5.9 (w/w %) of the nanobiologic composition.

In embodiments, the weight percentage of cholesterol in the nanobiologic compositions of the present disclosure ranges from about 0% (w/w) to about 15% (w/w) of cholesterol relative to phospholipids including from about 1% (w/w), about 1.5% (w/w), about 2% (w/w), about 2.5% (w/w), about 3% (w/w), about 3.5% (w/w), about 4% (w/w), about 4.5% (w/w), about 5% (w/w), about 5.5% (w/w), about 6% (w/w), about 6.5% (w/w), about 7% (w/w), about 7.5% (w/w), about 8% (w/w), about 8.5% (w/w), about 9% (w/w), about 9.5% (w/w), about 10% (w/w), about 10.5% (w/w), about 11% (w/w/), about 11.5% (w/w), about 12% (w/w), about 12.5% (w/w), about 13% (w/w), about 13.5% (w/w), about 14% (w/w), about 14.5% (w/w), to about 15% (w/w).

In embodiments, the size and circulating time of the nanoparticles can be modulated, for example, by controlling the ratio of lipids to ApoA-I and the ratio of lipids to polymer or lipids to triglyceride.

In embodiments, the nanobiologic composition comprises from about a 5:1 to 1000:1 ratio (e.g., on a molar basis) of phospholipids and/or sphingolipids: ApoA-I or a mimetic of apoA-I, including about 5:1, about 10:1, about 20:1, about 30:1, about 40:1, about 50:1, about 60:1, about 70:1, about 80:1, about 90:1, about 100:1, about 110:1, about 120:1, about 130:1, about 140:1, about 150:1, about 160:1, about 170:1, about 180:1, about 190:1, about 200:1, about 210:1, about 220:1 about 230:1, about 240:1, about 250:1, about 260:1, about 270:1, about 280:1, about 290:1, about 300:1, about 310:1, about 320:1, about 330:1, about 340:1, about 350:1, about 360:1, about 370:1, about 380:1, about 390:1, about 400:1, about 410:1, about 420:1, about 430:1, about 440:1, about 450:1, about 460:1, about 470:1, about 480:1, about 490:1, about 500:1, about 510:1, about 520:1, about 530:1, about 540:1, about 550:1, about 560:1, about 570:1, about 580:1, about 590:1, about 600:1, about 610:1, about 620:1, about 630:1, about 640:1, about 650:1, about 660:1, about 670:1, about 680:1, about 690:1, about 700:1, about 710:1, about 720:1 about 730:1, about 740:1, about 750:1, about 760:1, about 770:1, about 780:1, about 790:1, about 800:1, about 810:1, about 820:1, about 830:1, about 840:1, about 850:1, about 860:1, about 870:1, about 880:1, about 890:1, about 900:1, about 910:1, about 920:1, about 930:1, about 940:1, about 950:1, about 960:1, about 970:1, about 980:1, about 990:1, to about 1000:1, including all subranges and values therebetween.

In embodiments, the nanobiologic composition from about a 70:1 to 125:1 ratio (e.g., on a molar basis) of lipid:apoA-I. In embodiments, the HDL-derived the nanoparticle comprises from about a 5:1 to 10:1 ratio (e.g., on a molar basis) of lipid:mimetic of apoA-I.

In embodiments, the HDL-derived nanoparticle comprises from about a 2:1 to 3:1 ratio by weight of lipids:apoA-I or a mimetic of apoA-I.

In embodiments of the nanobiologic compositions provided herein, the phospholipid, sphingolipid, and cholesterol are present in a molar ratio of about 1:0.05-0.25:0.05-0.25.

In embodiments of the nanobiologic compositions provided herein, the phospholipid, sphingolipid, and cholesterol are present in a molar ratio of about 1:0.15-0.25:0.1-0.25.

In embodiments of the nanobiologic compositions provided herein, the phospholipid, sphingolipid, and cholesterol are present in a molar ratio of about 1:0.2:0.1-0.3.

In embodiments of the nanobiologic compositions provided herein, the phospholipid, the phospholipid, sphingolipid, and cholesterol are present in a molar ratio of about 1:0.2:0.2.

In embodiments, the nanobiologic composition comprises i) apoA-I or a peptide mimetic of apoA-I; ii) a phospholipid; iii) a lysophospholipid, and iv) cholesterol.

In embodiments, the nanobiologic composition comprises i) apoA-I or a peptide mimetic of apoA-I; ii) a phospholipid; and iii) cholesterol.

In embodiments, the nanobiologic composition comprises i) apoA-I or a peptide mimetic of apoA-I; ii) a phospholipid; iii) a lysophospholipid, iv) a hydrophobic matrix core and v) cholesterol.

In embodiments, the structure and properties of the HDL-derived nanoparticles (e.g., particle size, rigidity, viscosity, loading, etc.) can be modified by incorporating a hydrophobic matrix. As used herein, hydrophobic matrix refers to a core or filler or structural modifier of the nanobiologic. Non-limiting examples of suitable hydrophobic matrix molecules include, triglycerides, fatty acid esters, hydrophobic polymers, sterol esters, or combinations thereof.

Any suitable synthetic or natural fatty acid or fatty acid ester, known in the art are contemplated for use in the nanobiologic compositions of the present disclosure. Non-limiting examples of fatty acids of use include: arachidonic acid, oleic acid, arachidic acid, lauric acid, sad, capric acid, myristic acid, Palmic acid, stearic acid, linoleic acid, linolenic acid, dicaprate, three decanoins, glycerin mono-fatty acid ester, Dilaurin, 1-Sunsoft 767, laurocapram (1-dodecyl-aza-cycloheptane-2-ketone), acylcarnitines, acyl group choline or C₁-C₁₀arrcostab (such as isopropyl myristate IPM), monoglyceride, diglyceride or its pharmaceutically acceptable salt.

Any suitable synthetic or natural triglycerides, known in the art are contemplated for use in the nanobiologic compositions of the present disclosure. Non-limiting examples of triglycerides of use include: tricaprylin, tristearin, triolein, tripalmitin, 1,2-dipalmitoolein, 1,3-dipalmitoolein, 1-palmito-3-stearo-2-olein, 1-palmito-2-stearo-3-olein, 2-palmito-1-stearo-3-olein, trilinolein, 1,2-dipalmitolinolein, 1-palmito-dilinolein, 1-stearo-dilinolein, 1,2-diacetopalmitin, 1,2-distearo-olein, 1,3-distearo-olein, trimyristin, trilaurin and combinations thereof. Suitable triglycerides may be added to the present compositions in neat form. Additionally, or alternatively, oils and/or processed oils containing suitable triglycerides may be added to the compositions. Non-limiting examples of oils include coconut oil, corn germ oil, olive oil, palm seed oil, cottonseed oil, palm oil, rapeseed oil, sunflower oil, whale oil, soybean oil, peanut oil, linseed oil, tall oil, and combinations thereof.

In embodiments of the nanobiologic compositions provided herein, the nanobiologic composition has a PDI of about 0.01 to about 0.5, including about 0.01, about 0.02, about 0.03, about 0.04, about 0.05, about 0.06, about 0.07, about 0.08, about 0.09, about 0.1, about 0.2, about 0.3, about 0.4 to about 0.5, including all subranges and values therebetween.

In embodiments of the nanobiologic compositions provided herein, the nanobiologic composition has a PDI of about 0.1 to about 0.3.

In embodiments, the nanobiologic composition ranges from about 5 nm to about 400 nm in diameter, including about 5 nm, about 6 nm, about 7 nm, about 8 nm, about 9 nm, about 10 nm, about 11 nm, about 12 nm, about 13 nm, about 14 nm, about 15 nm, about 16 nm, about 17 nm, about 18 nm, about 19 nm, about 20 nm, about 21 nm, about 22 nm, about 23 nm, about 24, about 25 nm, about 26 nm, about 27 nm, about 28 nm, about 29, about 30 nm, about 31 nm, about 32 nm, about 33 nm, about 34, about 35 nm, about 36 nm, about 37 nm, about 38 nm, about 39, about 40 nm, about 41 nm, about 42 nm, about 43 nm, about 44 nm, about 45 nm, about 46 nm, about 47 nm, about 48 nm, about 49 nm, about 50 nm, about 51 nm, about 52 nm, about 53 nm, about 54 nm, about 55 nm, about 56 nm, about 57 nm, about 58 nm, about 59 nm, about 60 nm, about 61 nm, about 62 nm, about 63 nm, about 64, about 65 nm, about 66 nm, about 67 nm, about 68 nm, about 69 nm, about 70 nm, about 71 nm, about 72 nm, about 73 nm, about 74 nm, about 75 nm, about 76 nm, about 77 nm, about 78 nm, about 79 nm, about 80 nm, about 81 nm, about 82 nm, about 83 nm, about 84 nm, about 85 nm, about 86 nm, about 87 nm, about 88 nm, about 89, about 90 nm, about 91 nm, about 92 nm, about 93 nm, about 94, about 95 nm, about 96 nm, about 97 nm, about 98 nm, about 99 nm, about 100 nm, about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm, about 115 nm, about 116 nm, about 117 nm, about 118 nm, about 119 nm, about 120 nm, about 121 nm, about 122 nm, about 123 nm, about 124 nm, about 125 nm, about 126 nm, about 127 nm, about 128 nm, about 129 nm, about 130 nm, about 131 nm, about 132 nm, about 133 nm, about 134 nm, about 135 nm, about 136 nm, about 137 nm, about 138 nm, about 139 nm, about 140 nm, about 141 nm, about 142 nm, about 143 nm, about 144 nm, about 145 nm, about 146 nm, about 147 nm, about 148 nm, about 149 nm, about 150 nm, about 160 nm, about 170 nm, about 180 nm, about 190 nm, about 200 nm, about 210 nm, about 220 nm, about 230 nm, about 240 nm, about 250 nm, about 260 nm, about 270 nm, about 280 nm, about 290 nm, about 300 nm, about 310 nm, about 320 nm, about 330 nm, about 340 nm, about 350 nm, about 360 nm, about 370 nm, about 380 nm, about 390 nm, to about 400 nm including all ranges and values therebetween. In embodiments of the nanobiologic compositions provided herein, the nanobiologic composition is about, 5 nm to about 30 nm in diameter, 5 nm to about 150 nm in diameter, about 20 nm to about 150 nm in diameter, about 15 nm to about 250 nm in diameter, or about 305 nm to about 100 nm in diameter. In embodiments of the nanobiologic compositions provided herein, the nanobiologic composition is about 20 nm to about 100 nm in diameter. In embodiments of the nanobiologic compositions provided herein, the nanobiologic composition is about 25 nm to about 60 nm in diameter. In embodiments, the nanobiologic composition diameters are measured by dynamic light scattering (DLS).

In embodiments, the nanobiologic compositions of the present disclosure are discoidal.

In embodiments, the nanobiologic compositions of the present disclosure are spherical.

In embodiments, the nanobiologic compositions of the present disclosure comprise one or more promoters of trained immunity, or inhibitors of trained immunity inhibitors disclosed in US2019/0290593, US2020/0253884, US2020/0376146, WO2018/071549, and US2022-0332762 which are hereby incorporated by reference in their entireties for all purposes.

In embodiments, the nanobiologic compositions of the present disclosure comprise trained immunity promoter. In embodiments, the trained immunity promoter is a Dectin-1 receptor agonist. In embodiments, the trained immunity promoter is a NOD2 agonist, such as a muramyl dipeptide (MDP) a muramyl tripeptide (MTP) or a derivative or pro-drug thereof (e.g., mifamurtide). In embodiments, the trained immunity promoter is a derivative of muramyl dipeptide (MDP). In embodiments, the trained immunity promoter is a muramyl dipeptide phosphatidylethanolamine.

In embodiments, the nanobiologic composition comprises N—(N-Acetylmuramoyl)-L-alanyl-D-alpha-glutaminyl-N-[(7R)-4-hydroxy-4-oxido-10-oxo-7-[(1-oxohexadecyl)oxy]-3,5,9-trioxa-4-phosphapentacos-1-yl]-L-alaninamide.

In embodiments, the nanobiologic compositions of the present disclosure comprise trained immunity inhibitor. In embodiments, the trained immunity inhibitor is one or more trained immunity inhibitors disclosed in U.S. 2020/0376146, which is incorporated by reference herein. In embodiments, the trained immunity inhibitor is a mTOR inhibitor. In embodiments, the mTOR inhibitor is rapamycin or a prodrug thereof.

In embodiments, the mTOR inhibitor is CCL-779, RAD001 AP23573, C20-methallylrapamycin (C20-Marap), Cl6-(S) butysulfonamidorapamycin (Cl6-BSrap), Cl6-(S)-3-methylindolerapamycin (Cl6-iRap) (Bayle et al. Chemistry & Biology 2006, 13:99-107)), AZD8055, BEZ235 (NVP-BEZ235), chrysophanic acid chrysophanol), deforolimus (MK-8669), everolimus (RAD0001), KU-0063794, PI-103, PP242, temsirolimus, and WYE-354. In embodiments, the trained immunity inhibitor is a rapamycin derivative, e.g., disclosed in U.S. Pat. Nos. 5,665,772, 6,440,990, 5,985,890, or 6,200,985, each of which is hereby incorporated herein by reference. In embodiments, the compounds are 32-deoxorapamycin, 16-pent-2-ynyloxy-32-deoxorapambycin, 16-pent-2-ynyloxy-32 (S)-dihydro-rapamycin, 16-pent-2-ynyloxy-32 (S) dihydro-40-O-(2-hydroxyethyl)-rapamycin, or 40-O-(2-hydroxyethyl) rapamycin. In embodiments, the nanobiologic comprises a compound described in WO 94/09010, WO 95/16691 or WO 96/41807, and found to be useful e.g., as immunosuppressants.

In embodiments, sphingolipids of the present disclosure are formulated with any of the nanobiologic compositions disclosed in US2019/0290593, US2020/0253884, US2020/0376146 and WO2018/071549 which are hereby incorporated by reference in their entireties for all purposes.

### Pharmaceutical Formulations Containing Nanobiologic Compositions

When employed as pharmaceuticals, the nanobiologic compositions of the present disclosure are typically administered in the form of a pharmaceutical composition. Such compositions can be prepared in a manner well known in the pharmaceutical art and comprise at least one active compound. In some embodiments, the pharmaceutical composition comprises a nanobiologic composition of the present disclosure, and a pharmaceutically acceptable carrier. Pharmaceutical compositions disclosed herein may contain more than one type of nanobiologic; for example, a first nanobiologic may have a spherical structure and a second nanobiologic may have a discoidal structure.

Generally, the nanobiologic compositions of the disclosure are administered in a pharmaceutically effective amount. The amount of the compound actually administered will typically be determined by a physician, in the light of the relevant circumstances, including the condition to be treated, the chosen route of administration, the actual compound-administered, the age, weight, and response of the individual patient, the severity of the patient's symptoms, and the like.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions.

### Methods of Treatment

Provided herein are methods of treating a subject susceptible to or afflicted with immune-related diseases and conditions, including, for example, infectious diseases, immunoparalysis in sepsis and infections, cell proliferation disorders (such as cancer), autoimmune and autoinflammatory disorders, transplantation, cardiovascular diseases, neurodegenerative diseases, allergies and other immune-related diseases and conditions.

Examples of autoimmune disease include coeliac disease, type I diabetes, multiple sclerosis, thyroiditis, Grave's disease, systemic lupus erythematosus, scleroderma, psoriasis, arthritis, rheumatoid arthritis, alopecia greata, ankylosing spondylitis, Churg-Strauss Syndrome, autoimmune hemolytic anemia, autoimmune hepatitis, Behcet's disease, Crohn's disease, dermatomyositis, glomerulonephritis, Guillain-Barre syndrome, IBD, lupus nephritis, myasthenia gravis, myocarditis, pemphigus/pemphigoid, pernicious anemia, polyarteritis nodosa, polymyositis, primary biliary cirrhosis, rheumatic fever, sarcoidosis, Sjogren's syndrome, ulcerative colitis, uveitis, vitiligo, and Wegener's granulomatosis.

In embodiments, the nanobiologic compositions used herein may be used to treat an atherosclerotic lesion, such as coronary atherosclerosis, diabetic atherosclerosis, atherosclerosis, acute coronary syndrome myocardial infarction, angina pectoris, peripheral vascular disease, intermittent claudication.

In embodiments, the present disclosure provides methods for inducing transplant tolerance in a subject in need thereof, comprising administering to the subject in need thereof a therapeutically effective amount of a nanobiologic composition of the present disclosure. In embodiments, the present disclosure provides methods for the prophylaxis of organ or tissue rejection in a subject in need thereof, comprising administering to the subject a therapeutically effective amount of a nanobiologic composition of the present disclosure.

In embodiments, present disclosure provides methods for treating a subject with a viral, fungal, mycoplasma, bacterial, or protozoan infection, comprising administering to the subject a therapeutically effective amount of a nanobiologic composition of the present disclosure.

In embodiments, the present disclosure provides methods for improving the survival of an organ or tissue transplant in a patient in need thereof.

In embodiments, the transplanted tissue is lung tissue, heart tissue, kidney tissue, liver tissue, retinal tissue, corneal tissue, skin tissue, pancreatic tissue, intestinal tissue, genital tissue, ovary tissue, bone tissue, tendon tissue, bone marrow, or vascular tissue. In embodiments, the transplanted tissue is an intact organ. In embodiments, the subject is human, and the organ or tissue transplant is an allogeneic tissue or organ transplant. In embodiments, the nanobiologic composition is administered prior to, in conjunction with, or after the performance of an allogeneic tissue or organ transplant. In certain embodiments, the present method further comprises administering to the patient one or more immunosuppressant agents.

In embodiments, the present disclosure provides methods for treating cancer, comprising administering to a subject in need thereof a therapeutically effective amount of a nanobiologic composition of the present disclosure.

The cancer may be selected from the group consisting of bladder cancer, bone cancer, brain cancer (e.g., glioblastoma multiforme, glioma, astrocytoma), breast cancer, cervical cancer, colon cancer, endometrial cancer, esophageal cancer, eye cancer, head cancer, kidney cancer, liver cancer, lung cancer, mouth cancer, neck cancer, ovarian cancer, pancreatic cancer, prostate cancer (e.g., neuroendocrine prostate cancer), rectal cancer, colorectal cancer, gastric cancer, skin cancer, stomach cancer, testicular cancer, throat cancer, thyroid cancer, urothelial cancers, and uterine cancer.

In embodiments, the cancer is selected from the group consisting of bladder cancer, cancer of the blood vessels, bone cancer, brain cancer, breast cancer, cervical cancer, chest cancer, colon cancer, endometrial cancer, esophageal cancer, eye cancer, head cancer, kidney cancer, liver cancer, cancer of the lymph nodes, lung cancer, mouth cancer, neck cancer, ovarian cancer, pancreatic cancer, prostate cancer, rectal cancer, colorectal cancer, skin cancer, stomach cancer, testicular cancer, throat cancer, thyroid cancer, urothelial cancers, and uterine cancer.

In embodiments, the cancer is selected from the group consisting of breast cancer, prostate cancer, melanoma, colorectal cancer, lung cancer, pancreatic cancer, and glioblastoma.

The nanobiologic compositions of the present disclosure are contemplated for administration by a variety of routes including oral, rectal, intraocular, transdermal, subcutaneous, intravenous, intra-arterial, intramuscular, intraperitoneal, intradermal, directly into cerebrospinal fluid, intratracheal, and intranasal. Often, administration is intravenous.

The trained immunity promoter or trained immunity inhibitor (e.g., as disclosed herein) may be incorporated into the nanobiologic or may be physically discrete from the nanobiologic. When physically discrete the trained immunity promoter or trained immunity inhibitor and nanobiologic may be administered together, for example in a suspension, or may be administered separately by the same route of administration, or by different routes of administration. When administered separately, the trained immunity promoter or trained immunity inhibitor and nanobiologic may be administered within up to 20 minutes, up to an hour, up to 2 hour or within up to 1, 2, 3, 4, 5, 6,7, 8, 9, 10, 11, 12, 13, or 14 days of each other. In embodiments, the trained immunity promoter and nanobiologic may be administered at least 12 h, at least 1 day, at least two days, at least 3 days, at least 7 days or at least 2 weeks apart.

The compositions and methods disclosed herein provide two inputs into enhancing trained immunity. The sphingolipid containing nanobiologics compositions may also contain a trained immunity promoter or may be administered with a trained immunity promoter at a time-point sufficiently close to obtain the combined effect of both on the innate immune system. In embodiments, provided herein are methods of inducing or augmenting a trained immune response, the method comprising administering a nanobiologic composition of the present disclosure. In embodiments, inducing trained immunity is characterized by enhanced IL-6 production in sphingolipid nanobiologic treated PBMCs upon LPS stimulation compared to control PBMCs incubated with culture medium (RPMI) in an in vitro trained immunity assay e.g., as described herein. In embodiments, augmenting trained immunity is characterized by enhanced IL-6 production in sphingolipid nanobiologic and HKCA treated PBMCs upon LPS restimulation compared to control PBMCs treated with HKCA alone in an in vitro trained immunity assay e.g., as described herein.

In embodiments, provided herein are methods of suppressing a trained immune response, the method comprising administering a nanobiologic composition of the present disclosure. In embodiments, suppressing or inhibiting trained immunity is characterized by suppressed TNF production in sphingolipid nanobiologic treated PBMCs upon LPS restimulation compared to control PBMCs incubated with culture medium (RPMI) in an in vitro trained immunity assay e.g., as described herein. In embodiments, suppressing or inhibiting trained immunity is characterized by suppressed TNF and/or IL-6 production in sphingolipid nanobiologic and HKCA treated PBMCs upon LPS restimulation compared to control PBMCs treated with HKCA alone in an in vitro trained immunity assay e.g., as described herein.

In embodiments, the present disclosure provides a method of stimulating or inhibiting a trained immunity response in a subject comprising administering
(i) a nanobiologic composition comprising:
   (a) a apolipoprotein A-I (apoA-I) or a peptide mimetic of apoA-I;
   (b) a phospholipid; and
   (c) cholesterol;
   (d) a sphingolipid
   wherein the sphingolipid is present as about 1-50 mol % of the total lipid composition; and wherein said nanobiologic composition is between about 8 nm and about 150 nm in diameter; and
(ii) a trained immune promoter or inhibitor.

A nanobiologic composition described herein can be provided in a kit. In some embodiment the kit includes (a) a nanobiologic composition described herein, and, optionally (b) informational material. The informational material can be descriptive, instructional, marketing or other material that relates to the methods described herein and/or the use of a composition described herein for the methods described herein. In embodiments, the informational material can include information about production of the nanobiologic composition. In some embodiments, the informational material relates to methods for administering the nanobiologic composition. In embodiments, the informational material can include instructions to administer a nanobiologic composition described herein in a suitable manner to perform the methods described herein, e.g., in a suitable dose, dosage form, or mode of administration (e.g., a dose, dosage form, or mode of administration described herein). In some embodiments, the informational material can include instructions to administer a compound described herein to a suitable subject, e.g., a human, e.g., a human having or at risk for a disorder described herein.

The kit can include one or more containers for the composition containing a nanobiologic composition described herein. In some embodiments, the kit contains separate containers, dividers or compartments for the composition and informational material. For example, the composition can be contained in a bottle, vial, or syringe, and the informational material can be contained in a plastic sleeve or packet. In some embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a nanobiologic composition described herein.

In embodiments, provided herein is a nanobiologic composition prepared according to the methods disclosed herein.

### EXAMPLES

### Example 1: Formulating Sphingolipid-Loaded Nanobiologics

1,2-dimyristoyl-sn-glycero-3-phosphocholine (DMPC), cholesterol and sphingolipids were obtained from Avanti Lipids with a purity >99%. ApoA-1 was isolated from human HDL concentrate (Biosource Technology) as previously reported (Braza, M. S., et al., Immunity, 2018. 49 (5): p. 819-828 e6). DMPC (2.50 mg, 3.69 μmol, 1.0 eq.), cholesterol (0.29 mg, 0.74 μmol, 0.2 eq.), and sphingolipid (0.74 μmol, 0.2 eq.) were placed in a 20 mL vial and dissolved in chloroform (2.0 mL). For the unloaded-nanobiologics, used as controls, the sphingolipids were substituted by an additional 0.2 eq. DMPC. The solvent was evaporated under vacuum to create a lipid film, followed by the addition of apoA-1 (1.0 mg, 33 nM) in PBS (5.0 mL). The suspension was sonicated using a Branson Digital Sonifier SFX150 working at 60% power output for 7 minutes while being cooled in an ice-water bath. The slightly opaque solution was concentrated by centrifugal filtration using Vivaspin tubes (Sartorius Biotech, 10 kDa molecular weight cut-off at 4000 rpm and 4° C.) until a volume of approximately 1.0 mL remained. PBS (2.0 mL) was added and the sample again centrifuged until 1.0 mL remained; this was repeated once more. The resulting solution was filtered through a 0.22 μm polyethersulfone (PES) syringe filter (Celltreat) to obtain the sphingolipids-loaded nanobiologics as a white emulsion.

### Characterizing the Size and Sphingolipid-Loading of the Sphingolipid-Loaded Nanobiologics

Particle size was determined by dynamic light scattering (DLS) using a Brookhaven Instrument Corporation ZetaPALS analyzer. An aliquot (20 μL) of the nanobiologics was diluted with 1.0 mL PBS and filtered using a 0.22 μm PES syringe filter to remove any dust. Six separate runs of 1 minute each were recorded, and the mean of the number average size distribution reported. The sphingolipid concentration in the nanobiologic emulsions was determined by 1H NMR. An aliquot of the formulated sphingolipid-loaded nanobiologics (˜0.25 mL) was freeze-dried and resuspended in a mixture of deuterated chloroform and methanol (50:50 vol. %, 0.5 mL total) containing a known concentration of DMPC. The sample was analyzed by 1H NMR and the intensities of signals characteristic for DMPC (CH₂—COO) and sphingolipid (double bonds) used to determine the sphingolipid concentration.

nanobiologic compositions loaded with the sphingolipids listed in Table 3 below were prepared. Sizes are reported as the mean of the number average size distribution. Both nanoparticle size and dispersity index were determined by dynamic light scattering.

TABLE 3
Sphingolipid-loaded nanobiologics.

| | | | Dispersity | |
|---|---|---|---|---|
| | | Size | index | |
| | | (nm) of | of | |
| | Sphingolipid | Nano- | Nano- | |
| # | name | particle | particle | Structure |
| 1 | Sphingosine-1- phosphate (d18:1/16:0) | 29.8 | 0.239 | 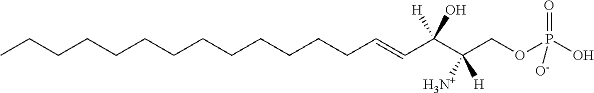 [SMILES: CCCCCCCCCCCCC/C=C/[C@@H](O)[C@@H]([NH3+])COP(=O)([O-])O] |
| 2 | Sphingosine (d20:1) | 45.3 | 0.196 | 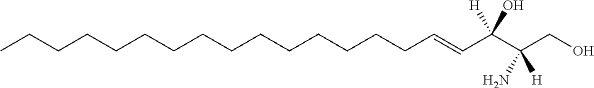 [SMILES: CCCCCCCCCCCCCCC/C=C/[C@@H](O)[C@@H](N)CO] |
| 3 | C24 Ceramide (d18:1/24:0) | 59.5 | 0.208 | 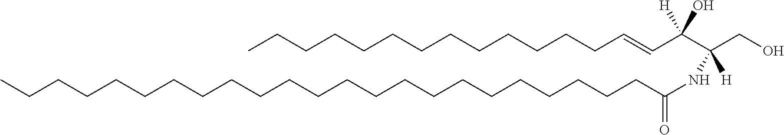 [SMILES: CCCCCCCCCCCCC/C=C/[C@@H](O)[C@H](CO)NC(=O)CCCCCCCCCCCCCCCCCCCCCCC] |
| 4 | C16 Ceramide (d18:1/16:0) | 32.7 | 0.242 | 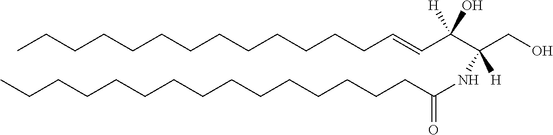 [SMILES: CCCCCCCCCCCCC/C=C/[C@@H](O)[C@H](CO)NC(=O)CCCCCCCCCCCCCCC] |
| 5 | C16 Dihydroceramide (18:0/16:0) | 59.7 | 0.165 | 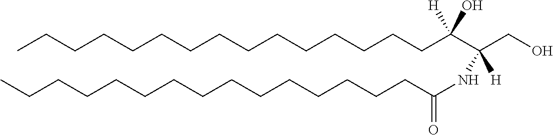 [SMILES: CCCCCCCCCCCCCCCC(=O)N[C@@H](CO)[C@H](O)CCCCCCCCCCCCCCC] |
| 6 | C24 Dihydroceramide (d18:0/24:0) | 47.3 | 0.180 | 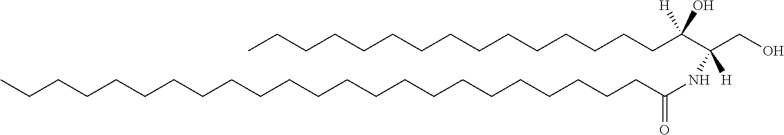 [SMILES: CCCCCCCCCCCCCCCCCCCCCCCC(=O)N[C@@H](CO)[C@H](O)CCCCCCCCCCCCCCC] |
| 7 | C16 Ceramide- 1-phosphate (d18:1/16:0) | 42.5 | 0.209 | 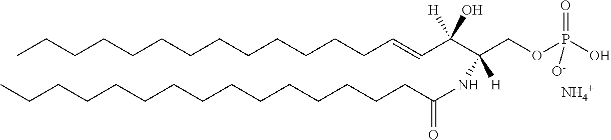 [SMILES: CCCCCCCCCCCCC/C=C/[C@@H](O)[C@H](COP(=O)([O-])O)NC(=O)CCCCCCCCCCCCCCC ; [NH4+]] |
| 8 | C18:1 Ceramide-1- phosphate (d18:1/18:1(9Z)) | 44.0 | 0.225 | 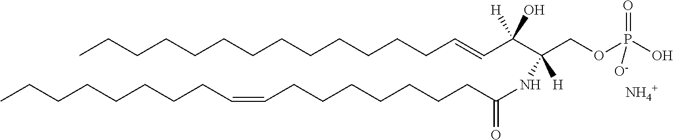 [SMILES: CCCCCCCC/C=C\CCCCCCCC(=O)N[C@@H](COP(=O)([O-])O)[C@H](O)/C=C/CCCCCCCCCCCCC ; [NH4+]] |
| 9 | C12 Ceramide- 1-phosphate (d18:1/12:0) | 34.0 | 0.251 | 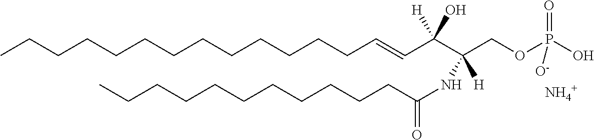 [SMILES: CCCCCCCCCCCCC/C=C/[C@@H](O)[C@H](COP(=O)([O-])O)NC(=O)CCCCCCCCCCC ; [NH4+]] |
| 10 | C16 Galactosyl(α) Ceramide (d18:1/16:0) | 35.7 | 0.226 | 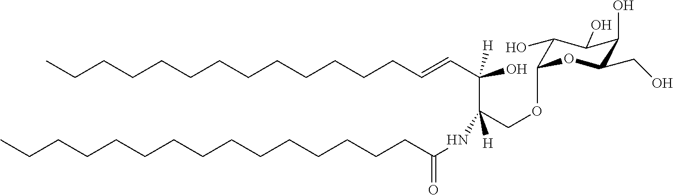 |
| 11 | C16 Galactosyl(β) Ceramide (d18:1/16:0) | 48.3 | 0.204 | 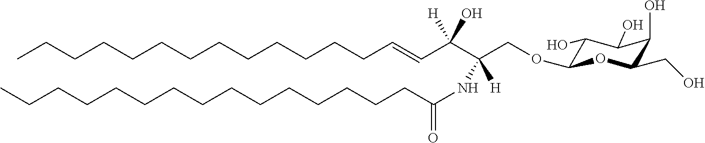 |
| 12 | C24:0 Galactosyl(β) Ceramide (d18:1/24:0) | 43.7 | 0.226 | 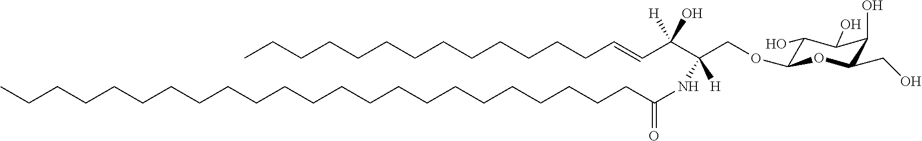 |
| 13 | C24:1 Galactosyl(β) Ceramide (d18:1/24:1) | 51.9 | 0.169 | 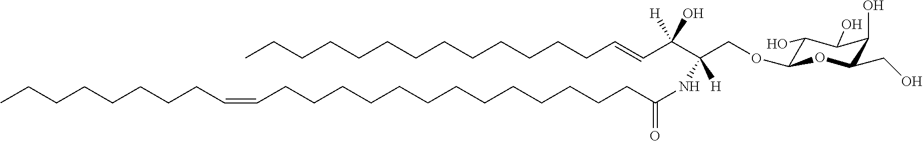 |
| 14 | C24:0 Lactosyl(β) Ceramide (d18:1/24:0) | 33.5 | 0.211 | 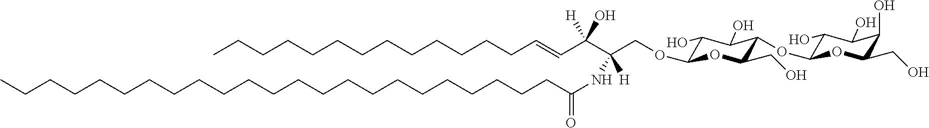 |
| 15 | C24:1 Lactosyl(β) Ceramide (d18:1/24:1) | 30.1 | 0.232 | 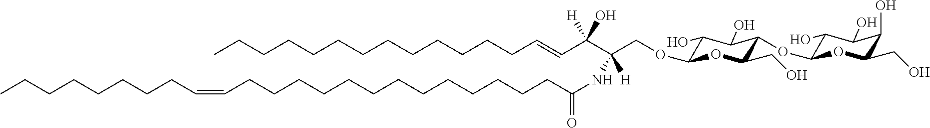 |
| 16 | 24:0 Sphingomyelin | 96.3 | 0.105 | 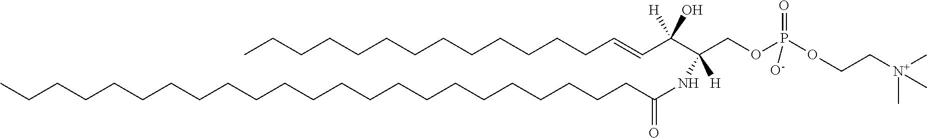 [SMILES: CCCCCCCCCCCCC/C=C/[C@@H](O)[C@H](COP(=O)([O-])OCC[N+](C)(C)C)NC(=O)CCCCCCCCCCCCCCCCCCCCCCC] |
| 17 | 24:1 Sphingomyelin | 40.6 | 0.217 | 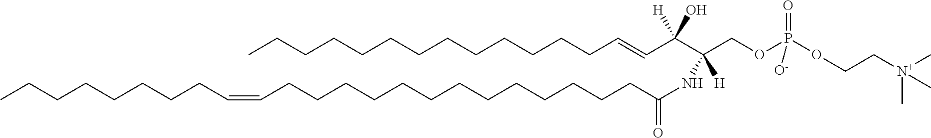 [SMILES: CCCCCCCC/C=C\CCCCCCCCCCCCCC(=O)N[C@@H](COP(=O)([O-])OCC[N+](C)(C)C)[C@H](O)/C=C/CCCCCCCCCCCCC] |
| 18 | C18:0 Glucosyl(β) Ceramide (d18:1/18:0) | 39.2 | 0.207 | 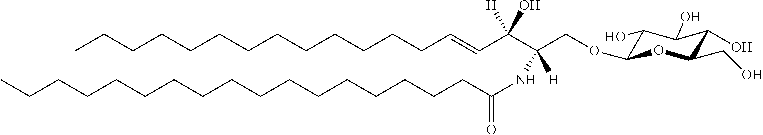 |
| 19 | C18:1 Glucosyl(β) Ceramide (d18:1/18:1(9Z)) | 41.1 | 0.184 | 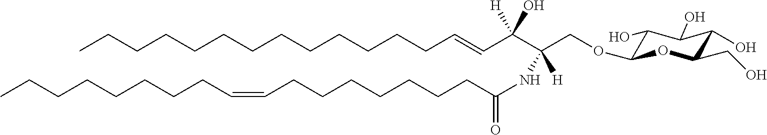 |
| Ctrl | Unloaded | 21.5 | 0.293 | NA |
| | nanobiologics | | | |

### Example 2: In Vitro Evaluation of the Effect of Exogenously Administered Sphingolipids on Innate Immune Memory in Human Primary Monocytes

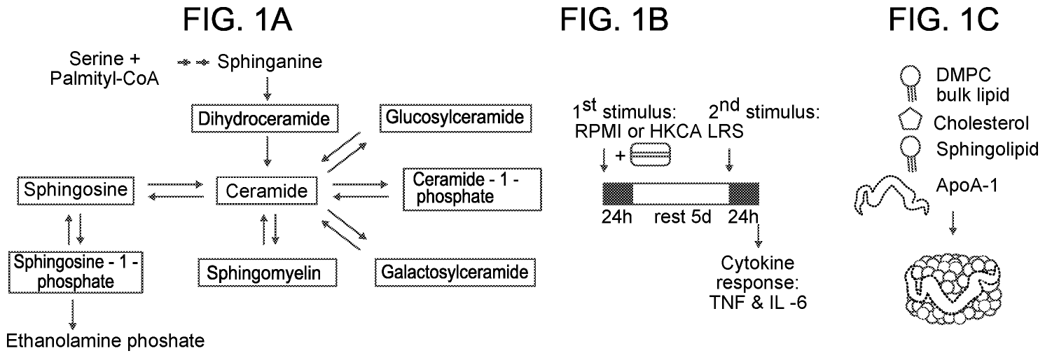

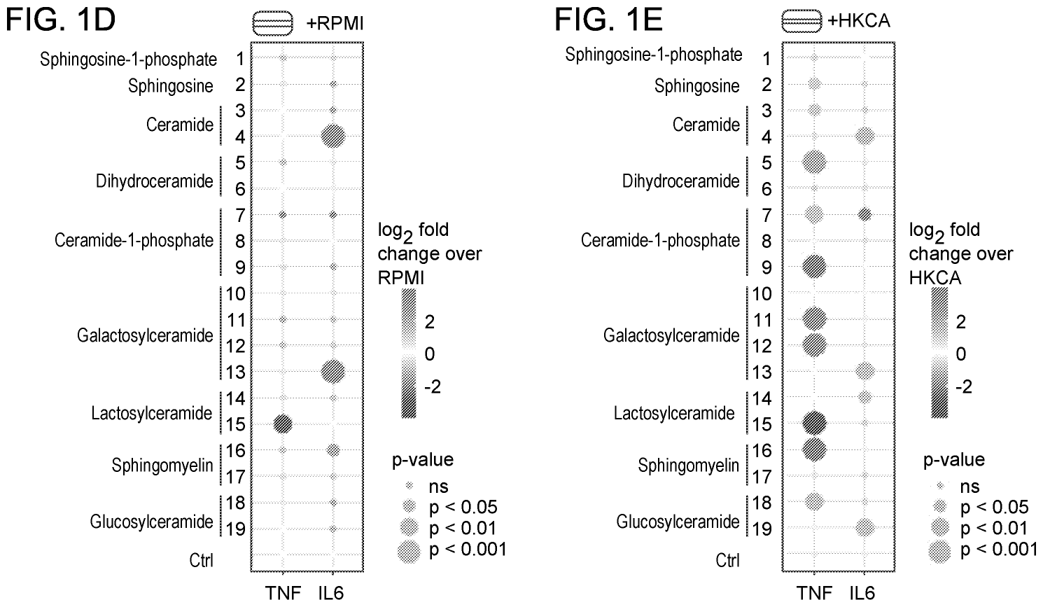

This study investigated the effect of exogenously administered sphingolipids on innate immune memory in human primary monocytes. To facilitate efficient delivery of sphingolipids to monocytes, the sphingolipids were formulated into the nanobiologic delivery platform described in Example 1 and FIG. 1C, which targets myeloid cells with high efficiency.

### Methods

### Human PBMC Isolation

PBMCs were isolated by differential centrifugation over Ficoll-Paque (Lymphoprep, StemCell Technologies, Inc.). Cells were washed two times in PBS. PBMCs were resuspended in RPMI culture medium supplemented with 2 mM glutamax, 1 mM pyruvate and penicillin/streptomycin (all from Thermo Fisher Scientific) and counted on a Casy counter (Innovatis).

### Lactate Dehydrogenase Measurements to Assess Cellular Toxicity

LDH concentration was measured in supernatants of PBMCs after 24-hour incubation with small molecule inhibitors or nanoparticles using CyQuant LDH Cytotoxicity Assay (Thermo Fisher Scientific). LDH concentration is calculated as percentage of maximal possible LDH concentration in completely lysed cells according to formula:

$$LDH ( \% of max ) = ( \frac{Compound induced LDH - Spontaneous LDH}{Maximum LDH - Spontaneous LDH} ) \times 100.$$

Training Experiments with Sphingolipid-Nanoparticles

Human PBMCs were isolated and plated as described above. After washing, cells were incubated with culture medium only as negative control, or treated with sphingolipid-loaded nanobiologics for 1 hour at 37° C. Cells were then incubated with 10⁵ cells/ml HKCA (Invivogen) together with the respective sphingolipid-loaded nanoparticle (50 μM) for 24 hours at 37° C. HKCA is a heat-killed preparation of C. albicans. HKCA activates the β-glucan specific dectin-1 receptor, which is expressed on phagocytes. Here, we used HKCA to promote trained immunity Subsequently, cells were washed, and rested for five days in RPMI culture medium containing 10% FBS. After the resting period cells were stimulated with either RPMI as negative control, 10 ng/ml LPS (Invivogen) or 1 μg/ml Pam3CSK4 (Invivogen).

### Monocyte Isolation

Monocytes were isolated using negative MACS isolation with the Pan monocyte isolation kit (Miltenyi Biotech). Briefly, stimulated PBMCs were washed with PBS and incubated with versene solution (0.48 mM EDTA, Sigma Aldrich) for 30 minutes at 37° C. Cells were scraped from the plates, counted, spun down and resuspended in MACS isolation buffer (PBS with 0.5% BSA and 2 mM EDTA). Monocyte isolation was performed according to manufacturer's instructions.

### Cytokine Measurements

Cytokine production was measured in supernatants using commercial ELISA kits for human TNF, IL-6, IFNγ and IL-1ß (R&D systems) according to manufacturer's instruction.

### Results

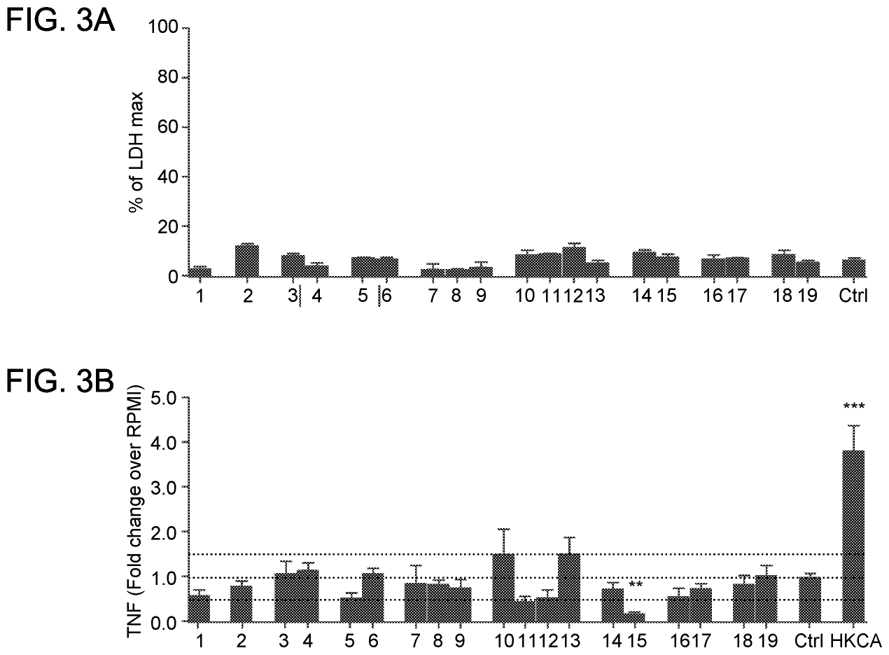

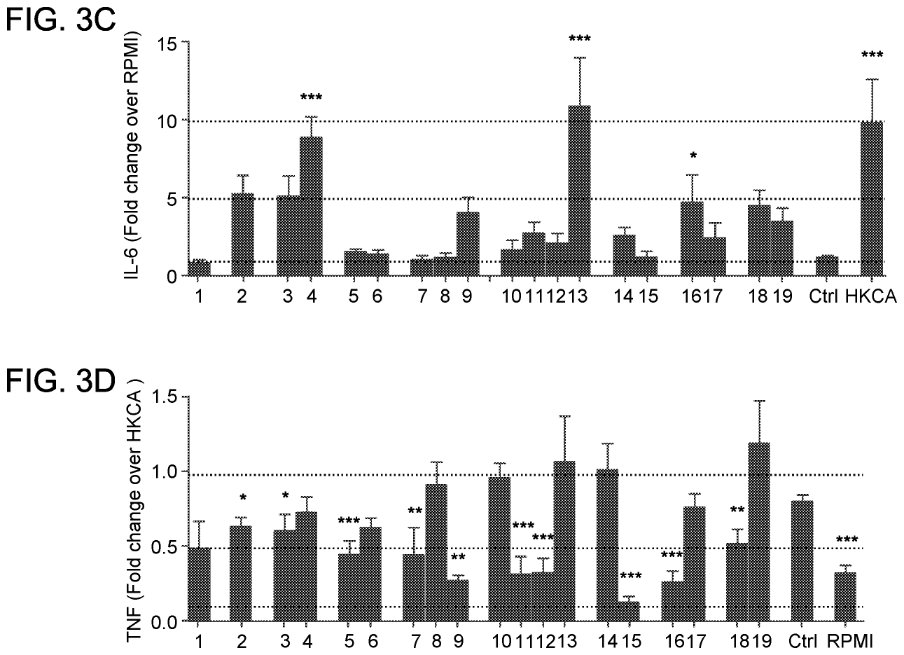

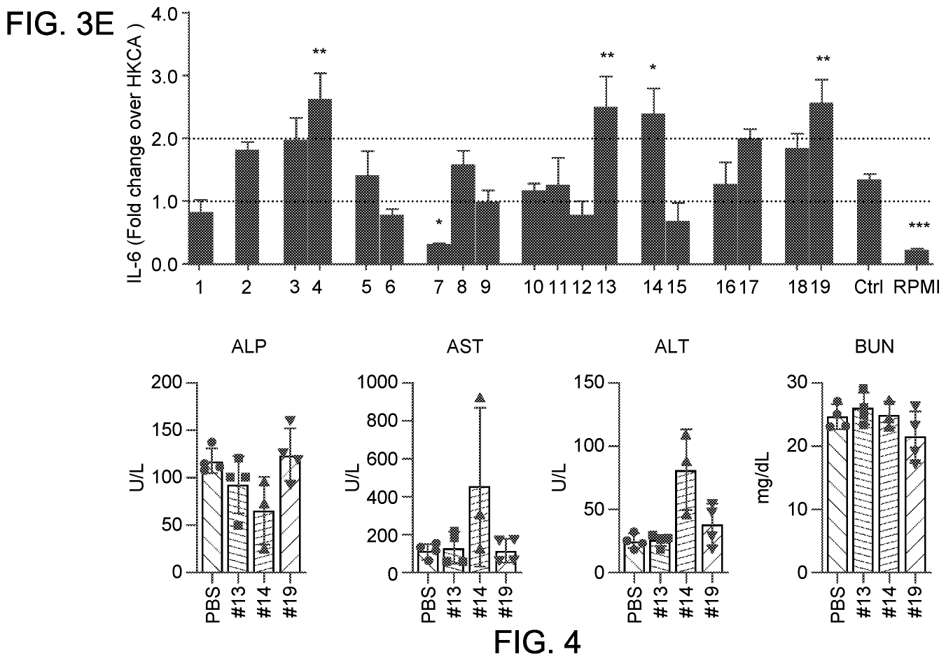

All tested nanobiologic formulations were non-toxic to PBMCs at the concentrations used (FIG. 3A).

PBMCs were either stimulated with sphingolipid-nanobiologics alone (FIG. 1D, FIG. 3B and FIG. 3C) or in combination with HKCA (FIG. 1E, FIG. 3D and FIG. 3E) for 24 hours and restimulated with LPS five days later.

Three sphingolipid-nanobiologics (d18:1/16:0 ceramide (Table 3, #4), d18:1/24:1 galactosyl(ß) ceramide (Table 3, #13) and 24:0 sphingomyelin (Table 3, #16)) enhanced the IL-6 production upon restimulation, indicating these nanobiologics induced trained immunity (FIG. 1D, FIG. 3C). Four sphingolipid-nanobiologics (d18:1/16:0 ceramide (Table 3, #4), d18:1/24:1 galactosyl(ß) ceramidase (Table 3, #13), d18:1/24:0 lactosyl (B) ceramide (Table 3, #14), and d18:1/18:1 glucosyl(ß) ceramide (Table 3, #19)) augmented the HKCA induced trained immune response for IL-6 (FIG. 1E, FIG. 3E).

Other sphingolipid-nanobiologics had the opposite effect. d18:1/24:1 lactosyl (ß) ceramide (Table 3, #15) suppressed the TNF response upon LPS stimulation five days later (FIG. 1D, FIG. 3C). Nine sphingolipid-nanobiologics (20:1 sphingosine (Table 3, #2), d18:1/24:0 ceramide (Table 3, #3), d18:0/16:0 dihydroceramide (Table 3, #5), d18:1/12:0 ceramide-1-phosphate (Table 3, #9), d18:1/16:0 and d18:1/24:0 galactosyl(ß) ceramide (Table 3, #11 and #12), d18:1/24:1 lactosyl (B) ceramide (Table 3, #15), 24:0 sphingomyelin (Table 3, #16) and d18:1/18:0 glucosyl(ß) ceramide (Table 3, #18)) inhibited the HKCA induced trained immune response for TNF. 18:1/16:0 ceramide-1-phosphate (Table 3, #7) suppressed HKCA-training for both the TNF and IL-6 response (FIG. 1E, FIG. 3D and FIG. 3E).

These data indicate that exogenously administered sphingolipid loaded nanobiologic compositions can affect innate immune memory. Even a short exposure to exogenously administered sphingolipid loaded nanobiologic compositions can induce long-term changes (e.g., about a week, about a month or more) in the function of innate immune cells, indicative of a trained immunity response. Interestingly, this process depends not only on the type of sphingolipid head-group, but also the saturation status of its fatty acid residue. Species with a saturated fatty acid chain (Table 3, #12, #14, #18) had an opposite effect on innate immune memory compared to their cis-monounsaturated counterparts (Table 3, #13, #15, #19). Additionally, inhibition of trained immunity by sphingolipid-nanobiologics had stronger effects on TNF production compared to IL-6 secretion. In contrast, induction of trained immunity was most clearly observed in the IL-6 response.

### Example 3: In Vivo Evaluation of Sphingolipid Nanobiologic Composition in a B16F10 Mouse Melanoma Model

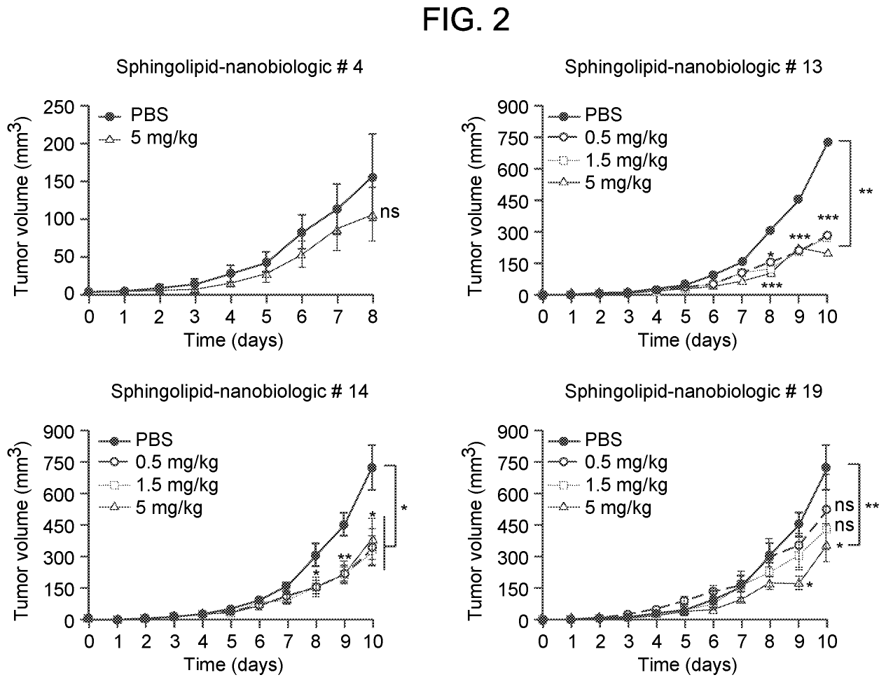

In this study nanobiologic formulations containing sphingolipids C_{24:1} Galactosyl(ß) Ceramide (d18:1/24:1 (15Z)) (#13), C16 Ceramide (d18:1/16:0) (d18:1/24:0) (#4), C24:0 Lactosyl(ß) Ceramide (d18:1/24:0) (#14) and C18:1 Glucosyl(ß) Ceramide (d18:1/18:1 (9Z)) (#19) were tested in a B16F10 melanoma model. In all cases, tumors were inoculated on day-7 and animals treated on days 0, 2, 4 (n=10) with intravenous injections of sphingolipid nanobiologics at doses of 0.5, 1.5, 5.0 mg sphingolipid/kg mouse. The black asterisks indicate significance in tumor size on that specific day, as determined by one-way Anova with Dunnett's multiple comparison analysis. The asterisks in rectangular box indicate significance in tumor growth rate. P values were calculated using a Mann-Whitney U test or an unpaired t-test. * p<0.05, ** p<0.01, *** p<0.001, ns=not significant as determined as described by Hather et. al, Cancer Inform. 2014, 134, 13974. The results are shown in FIG. 2. The data show significant tumor growth reducing properties of sphingolipid nanobiologics containing Galactosyl(ß) Ceramide (d18:1/24:1 (15Z)) (#13) ** p<0.01; C24:0 Lactosyl(ß) Ceramide (d18:1/24:0) (#14) *p<0.05; and C18:1 Glucosyl(ß) Ceramide (d18:1/18:1 (9Z)) ** p<0.01 (#19) and at 3 different doses.

Naïve C57BL/6 mice were given nanobiologics i.v. on days 0, 2, and 4 at a dose of 0.5 mg/kg for nanobiologic formulations #13 and #14 and 5 mg/kg for #19. Two days after the last injection, the animals were sacrificed, and blood chemistry analyses performed. Results are displayed in FIG. 4 and showed that formulations #13 and #19 are highly biocompatible, while formulations #14 induced mild liver toxicity, as indicated by elevated AST and ALT levels. ALP=alkaline phosphatase, AST=aspartate aminotransferase, ALT=alanine transaminase, BUN=blood urea nitrogen, n=4.

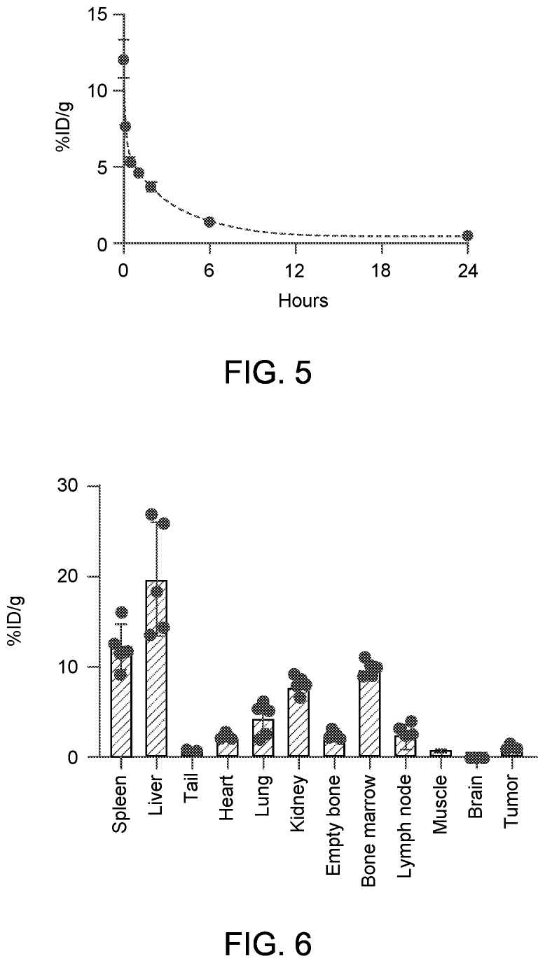

Formulation #13 was radiolabeled with ⁸⁹Zr (using the procedure reported in van Leent, M. M. T. et al. Sci Adv 7, 1-12, 2021) and i.v. administered to B16F10 melanoma-bearing mice. The radiolabeled nanobiologics' were subsequently assessed for pharmacokinetics (FIG. 5) and biodistribution (FIG. 6) at 24 hours by ex vivo gamma counting, (n=5).

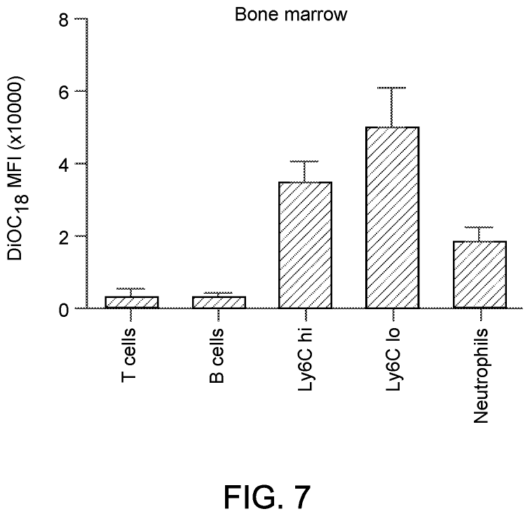

Formulation #13 loaded with the lipophilic fluorophore DiOC18 was injected i.v. in the B16F10 melanoma mouse model and the animals' bone marrow analyzed by flow cytometry after 24 hours (n=5) (FIG. 7). Results show that nanobiologic loaded with formulation #13 display high uptake in myeloid cells and the hematopoietic organs, (n=5). Data are mean±SD and mean±SEM for tumor growth experiments.

### EMBODIMENTS

1. A nanobiologic composition comprising:
   (a) a apolipoprotein A-I (apoA-I) or a peptide mimetic of apoA-I;
   (b) a sphingolipid; and
   (c) cholesterol;
   wherein the composition is a nanoparticle having a diameter between about 8 nm and about 150 nm.
2. The nanobiologic composition of embodiment 1, wherein the sphingolipid is selected from the group consisting of a ceramide, sphingomyelin, dihydroceramide, glucosylceramide, sphingosine, sphingosine-1-phosphate, galactosylceramide, ceramide-1-phosphate, lactosylceramide, and mixtures thereof.
3. The nanobiologic composition of embodiment 1 or 2, wherein the sphingolipid is selected from the group consisting of:

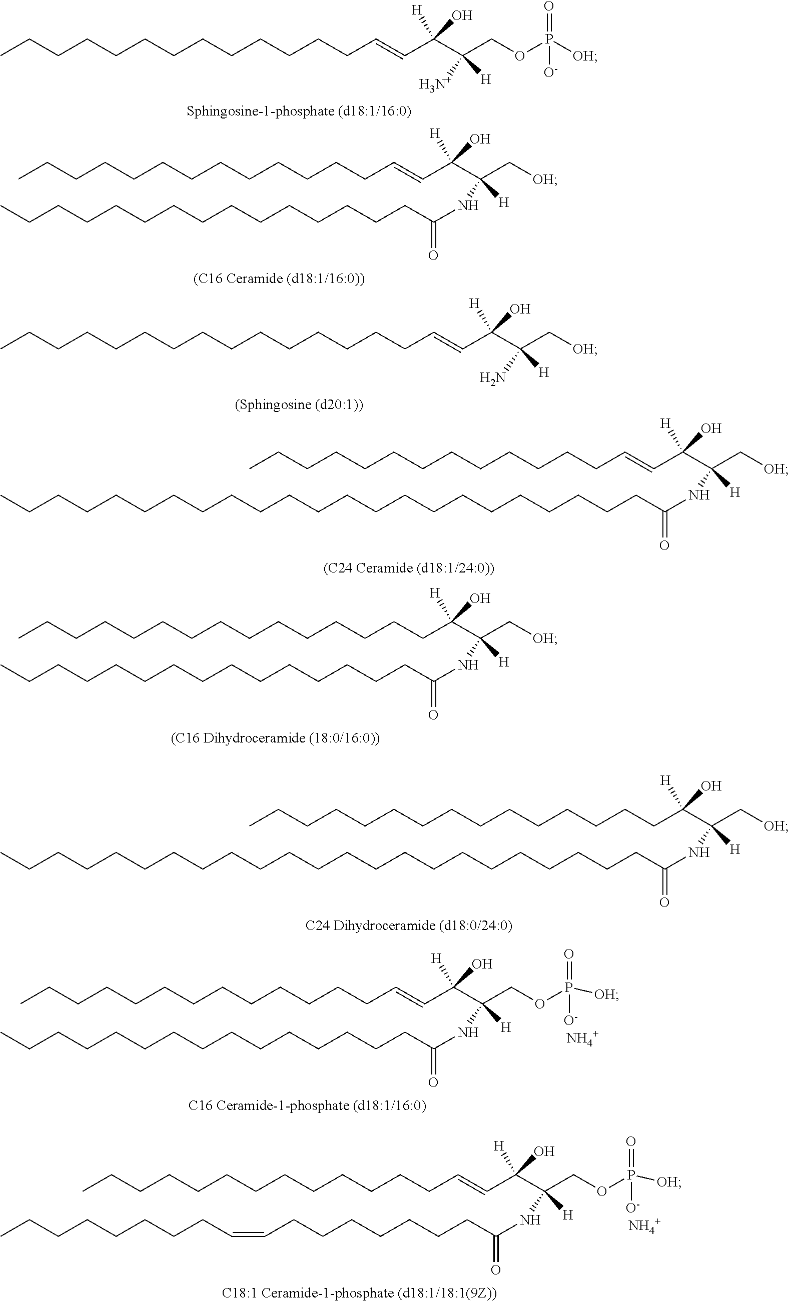

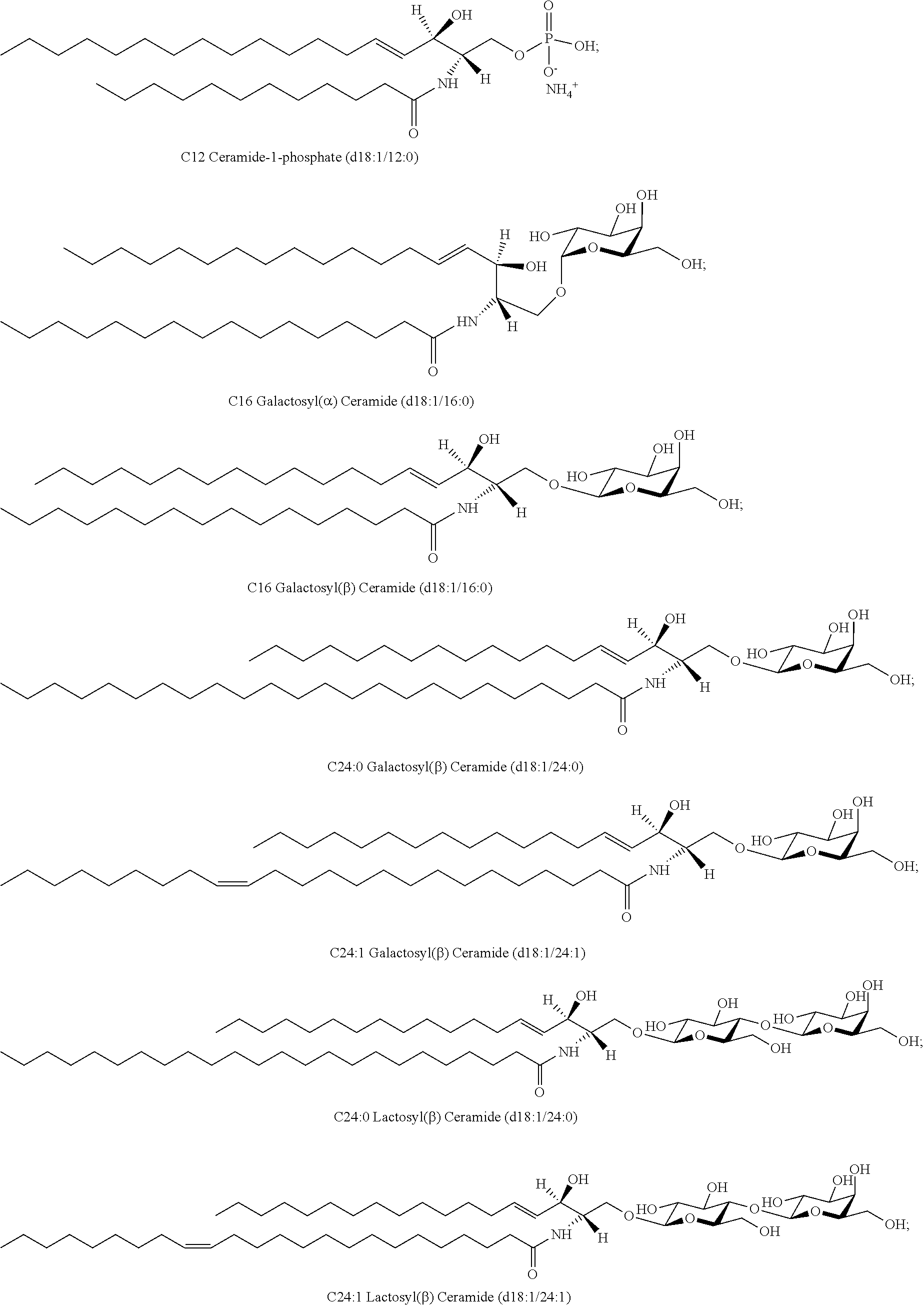

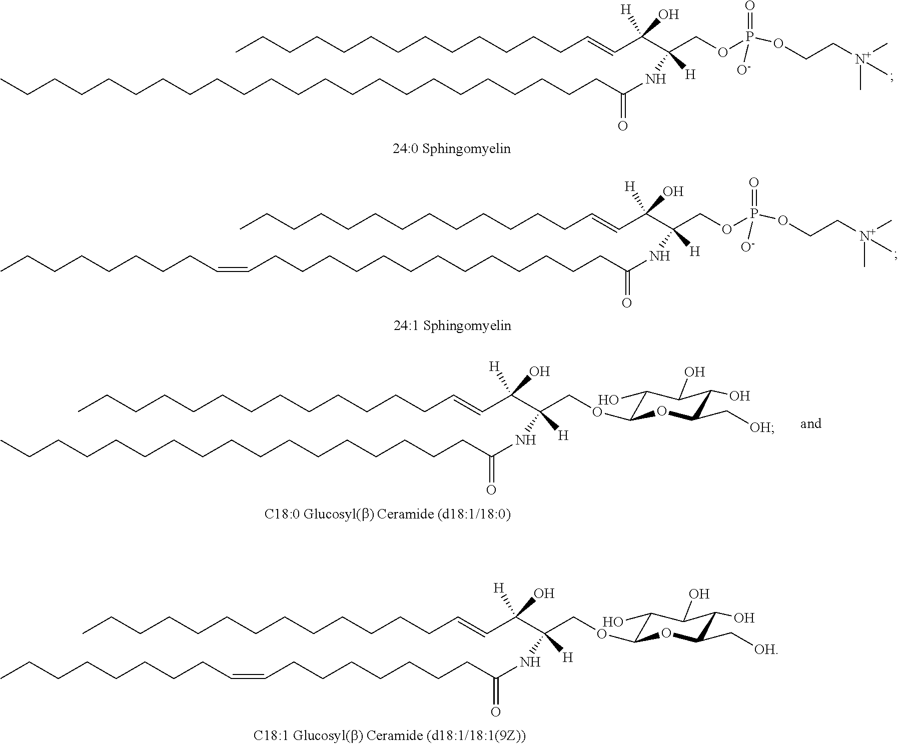

4. The nanobiologic composition of embodiment 3, wherein the sphingolipid is selected from the group consisting of:

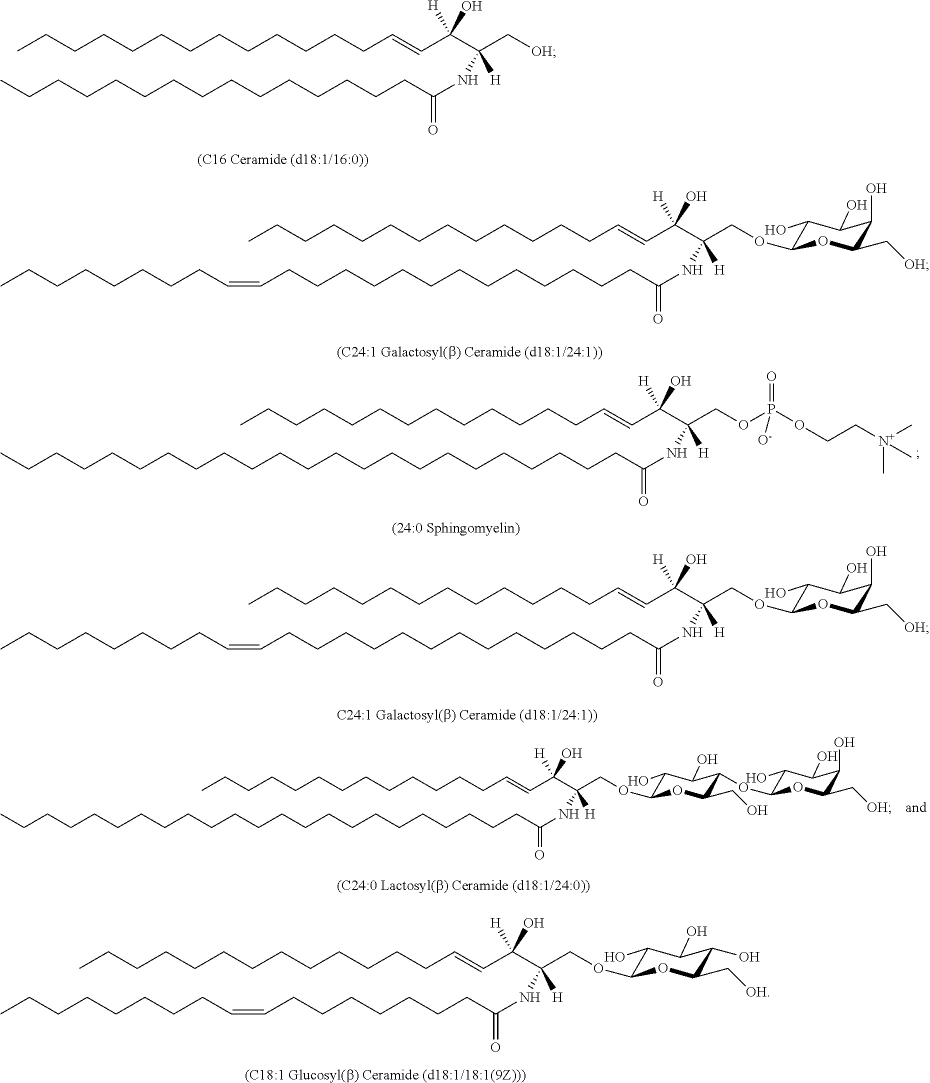

5. The nanobiologic composition of embodiment 3, wherein the sphingolipid is selected from the group consisting of:

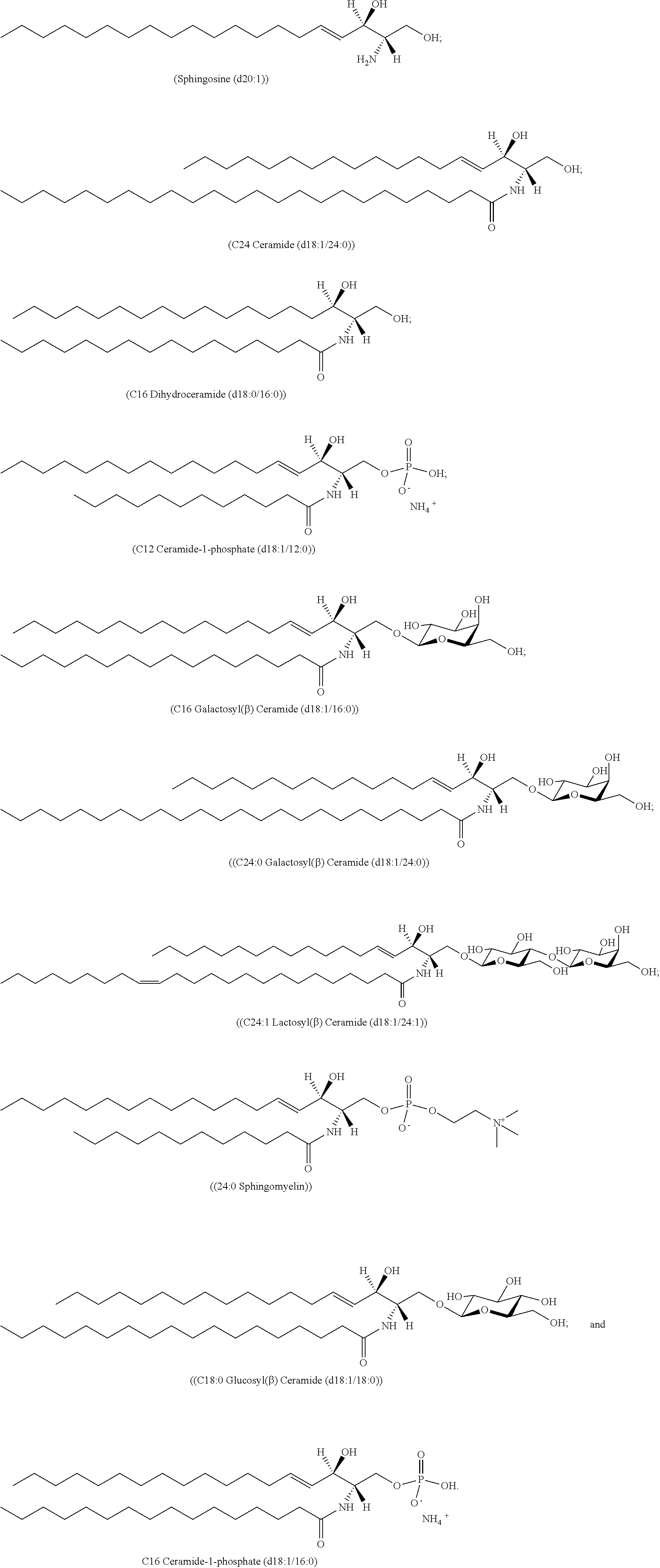

6. The nanobiologic composition of any one of embodiments 1-5, comprising a phospholipid.
7. The nanobiologic composition of embodiment 6, wherein the phospholipid is selected from the group consisting of 1,2-dimyristoyl-sn-glycero-3-phosphocholine (DMPC), 1-palmitoyl-2-oleoyl-sn-glycero-3-phosphocholine (POPC), 1,2-dioleoyl-sn-glycero-3-phosphocholine (DOPC) and mixtures thereof.
8. The nanobiologic composition of embodiment 7, wherein the phospholipid is DMPC.
9. The nanobiologic composition of any one of embodiments 1-5, comprising a phospholipid and a lysophospholipid.
10. The nanobiologic composition of any one of embodiments 1-9, wherein the phospholipid is 1,2-dimyristoyl-sn-glycero-3-phosphocholine (DMPC) or 1-palmitoyl-2-oleoyl-sn-glycero-3-phosphocholine (POPC).
11. The nanobiologic composition of embodiment 9, wherein the lysophospholipid is selected from the group consisting of 1-myristoyl-2-hydroxy-sn-glycero-3-phosphocholine (MHPC), 1-palmitoyl-2-hydroxy-sn-glycero-3-phosphocholine (PHPC), 1-stearoyl-2-hydroxy-sn-glycero-3-phosphocholine (SHPC), and mixtures thereof.
12. The nanobiologic composition of embodiment 9, wherein the phospholipid is 1-palmitoyl-2-oleoyl-sn-glycero-3-phosphocholine (POPC) and the lysophospholipid is 1-palmitoyl-2-hydroxy-sn-glycero-3-phosphocholine (PHPC).
13. The nanobiologic composition of any one of embodiments 1-12, wherein the nanobiologic composition comprises human apolipoprotein A-I (apoA-I).
14. The nanobiologic composition of any one of embodiments 6-13, wherein the sphingolipid is present in about 1-50 mol % of the total lipid composition.
15. The nanobiologic composition of embodiment 14, wherein the sphingolipid is present in about 10-25 mol % of the total lipid composition.
16. The nanobiologic composition of embodiment 15, wherein the sphingolipid is present in about 20 mol % of the total lipid composition.
17. The nanobiologic composition of any one of embodiments 1-16, wherein the cholesterol is present in about 1-30 mol % relative to the phospholipid.
18. The nanobiologic composition of embodiment 17, wherein the cholesterol is present in about 5-25 mol % relative to the phospholipid.
19. The nanobiologic composition of any one of embodiments 1-18, wherein the phospholipid, sphingolipid, and cholesterol are present in a molar ratio of about 1:0.05-0.25:0.05-0.25.
20. The nanobiologic composition of any one of embodiments 1-19, wherein the nanobiologic composition has a PDI of about 0.1 to about 0.3.
21. The nanobiologic composition of any one of embodiments 1-20, wherein the nanoparticle has a diameter between about 20 nm to about 100 nm.
22. The nanobiologic composition of embodiment 21, wherein the nanoparticle has a diameter between about 25 nm to about 60 nm.
23. The nanobiologic composition of any one of embodiments 1-22, wherein the nanoparticle is spherical.
24. The nanobiologic composition of any one of embodiments 1-22, wherein the nanoparticle is discoidal.
25. The nanobiologic composition of any one of embodiments 1-24, wherein the nanobiologic composition is suitable for intravenous or intra-arterial administration.
26. A method for treating a cancer in a subject in need thereof, comprising administering to the subject an effective amount of a nanobiologic composition of any one of embodiments 1-25.
27. The method of embodiment 26, wherein the cancer is selected from the group consisting of bladder cancer, cancer of the blood vessels, bone cancer, brain cancer, breast cancer, cervical cancer, chest cancer, colon cancer, endometrial cancer, esophageal cancer, eye cancer, head cancer, kidney cancer, liver cancer, cancer of the lymph nodes, lung cancer, mouth cancer, neck cancer, ovarian cancer, pancreatic cancer, prostate cancer, rectal cancer, colorectal cancer, skin cancer, stomach cancer, testicular cancer, throat cancer, thyroid cancer, urothelial cancers, and uterine cancer.
28. The method of embodiment 27, wherein the cancer is selected from the group consisting of breast cancer, prostate cancer, melanoma, colorectal cancer, lung cancer, pancreatic cancer, and glioblastoma.
29. The method of any one of embodiments 26-28, wherein the nanobiologic composition is administered intravenously or intra-arterially.
30. The method of any one of embodiments 26-29, wherein the subject is a human.
31. A method of stimulating a trained immunity response in a subject comprising administering
   (i) a nanobiologic composition of any one of embodiments 1-25; and
   (ii) a trained immune promoter.
32. The method of embodiment 31 wherein the trained immunity promoter is a Dectin-1 receptor agonist, a NOD-2 agonist, or a combination thereof.
33. The method of embodiment 31 wherein the nanobiologic composition comprises the trained immunity promoter.
34. The method of embodiment 31 wherein the nanobiologic composition and the trained immunity promoter are administered in separate compositions.
35. The method of embodiment 34 wherein the nanobiologic composition and trained immunity promoter are administered within up to 1, 2, 3, 4, 5, 6, or 7 days of each other.
36. The method of embodiment 34 or 35 wherein the routes of administration are the same.
37. The method of embodiment 36 wherein the route is intravenous, 38. The method of embodiment 34 or 35 wherein the routes of administration are different.
39. The method of embodiment 38 wherein the nanobiologic composition is administered intravenously.
40. The method of embodiment 31 wherein the trained immunity promoter is a Dectin-1 receptor agonist.
41. The method of embodiment 40 wherein the Dectin-1 receptor agonist is an HKCA (heat-killed Candida albicans).
42. The method of embodiment 31 wherein the trained immunity promoter is a NOD-2 agonist.
43. The method of embodiment 42 wherein the NOD2-agonist is a muramyl dipeptide (MDP) a muramyl tri-peptide (MTP) or a derivative or pro-drug thereof

1. A nanobiologic composition comprising:
(a) a apolipoprotein A-I (apoA-I) or a peptide mimetic of apoA-I;
(b) a sphingolipid; and
(c) cholesterol;
wherein the composition is a nanoparticle having a diameter between about 8 nm and about 150 nm.

2. The nanobiologic composition of claim 1, wherein the sphingolipid is selected from the group consisting of a ceramide, sphingomyelin, dihydroceramide, glucosylceramide, sphingosine, sphingosine-1-phosphate, galactosylceramide, ceramide-1-phosphate, lactosylceramide, and mixtures thereof.

3. The nanobiologic composition of claim 2, wherein the sphingolipid is selected from the group consisting of a glucosylceramide, galactosylceramide, or lactosylceramide.

4. The nanobiologic composition of claim 3, wherein the sphingolipid is a glucosylceramide or a galactosylceramide.

5. The nanobiologic composition of claim 4, wherein the sphingolipid is a glucosylceramide.

6. The nanobiologic composition of claim 4, wherein the sphingolipid is a galactosylceramide.

7. The nanobiologic composition of claim 1, wherein the sphingolipid is of Formula (I): 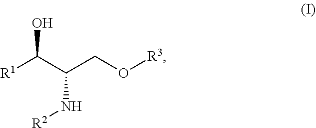
or a pharmaceutically acceptable salt thereof, wherein:
R¹ is a saturated aliphatic chain or an unsaturated aliphatic chain;
R² is H, R⁴, or C(O)R⁴;
R³ is H, a sugar, P(O)(OH)₂, or P(O)(OH)O(CH₂)ₙR⁵ where n=1-10;
R⁴ is a saturated aliphatic chain or an unsaturated aliphatic chain; and
R⁵ is H, NH₂, N(CH₃)₃⁺, OH, or a sugar.

8. The nanobiologic composition of claim 7, wherein
R¹ is a unsaturated C₁₄₋₂₄ aliphatic chain.
R² is C(O)R⁴;
R³ is 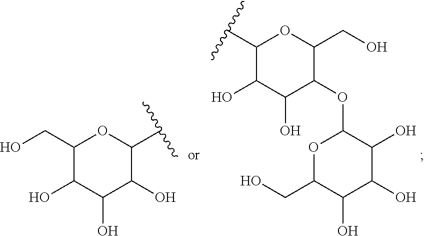 [SMILES: *C1OC(CO)C(O)C(O)C1O ; *C1OC(CO)C(OC2OC(CO)C(O)C(O)C2O)C(O)C1O]
and
R⁴ is a C₁₁₋₃₀ unsaturated aliphatic chain or a C₁₁₋₃₀ saturated aliphatic chain.

9. The nanobiologic composition of claim 8, wherein R¹ is a unsaturated C₁₅₋₁₇ aliphatic chain.

10. The nanobiologic composition of claim 9, wherein
R¹ is a unsaturated C₁₅₋₁₇ aliphatic chain;
R² is C(O)R⁴;
R³ is 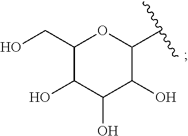 [SMILES: *C1OC(CO)C(O)C(O)C1O]
and
R⁴ is a C₁₁₋₃₀ unsaturated aliphatic chain.

11. The nanobiologic composition of any one of claims 7-10, wherein R¹ is a Cis alkenyl.

12. The nanobiologic composition of any one of claims 7-11, wherein R⁴ is a C₁₄₋₂₄ unsaturated aliphatic chain or a C₁₄₋₂₄ saturated aliphatic chain.

13. The nanobiologic composition of claim 12, wherein R⁴ is a C₁₇ alkenyl or a C₂₃ alkenyl.

14. The nanobiologic composition of any one of claims 7-13, wherein R³ is

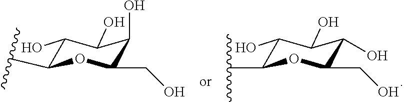

15. The nanobiologic composition of claim 1 or 2, wherein the sphingolipid is selected from the group consisting of:

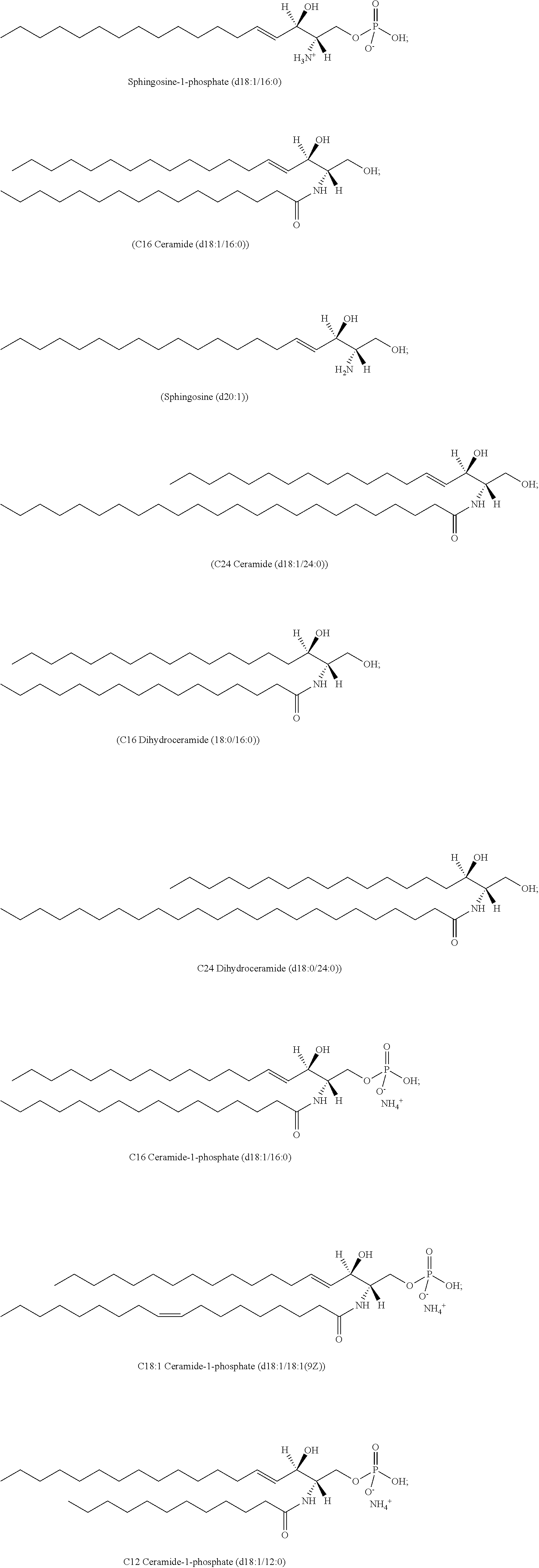

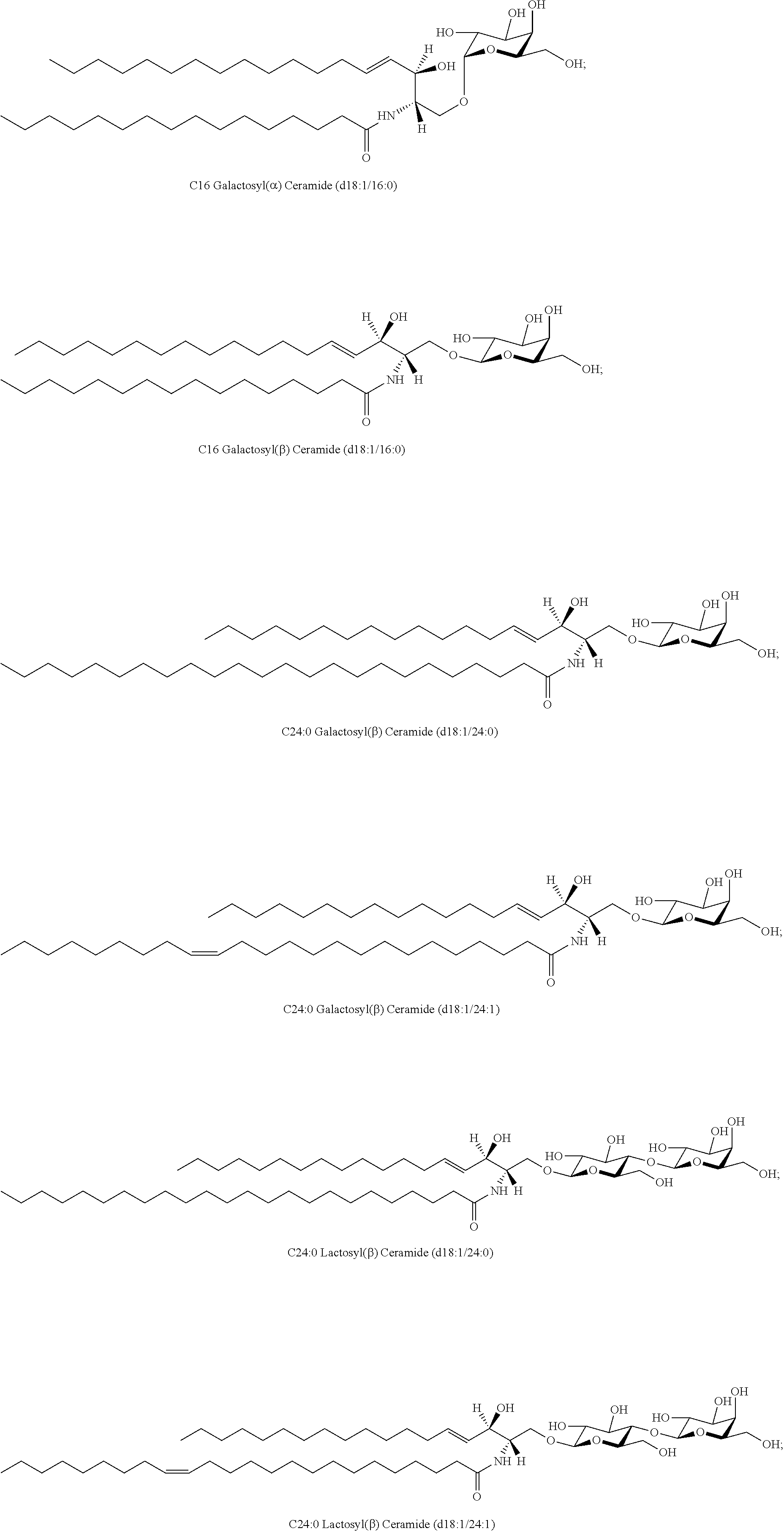

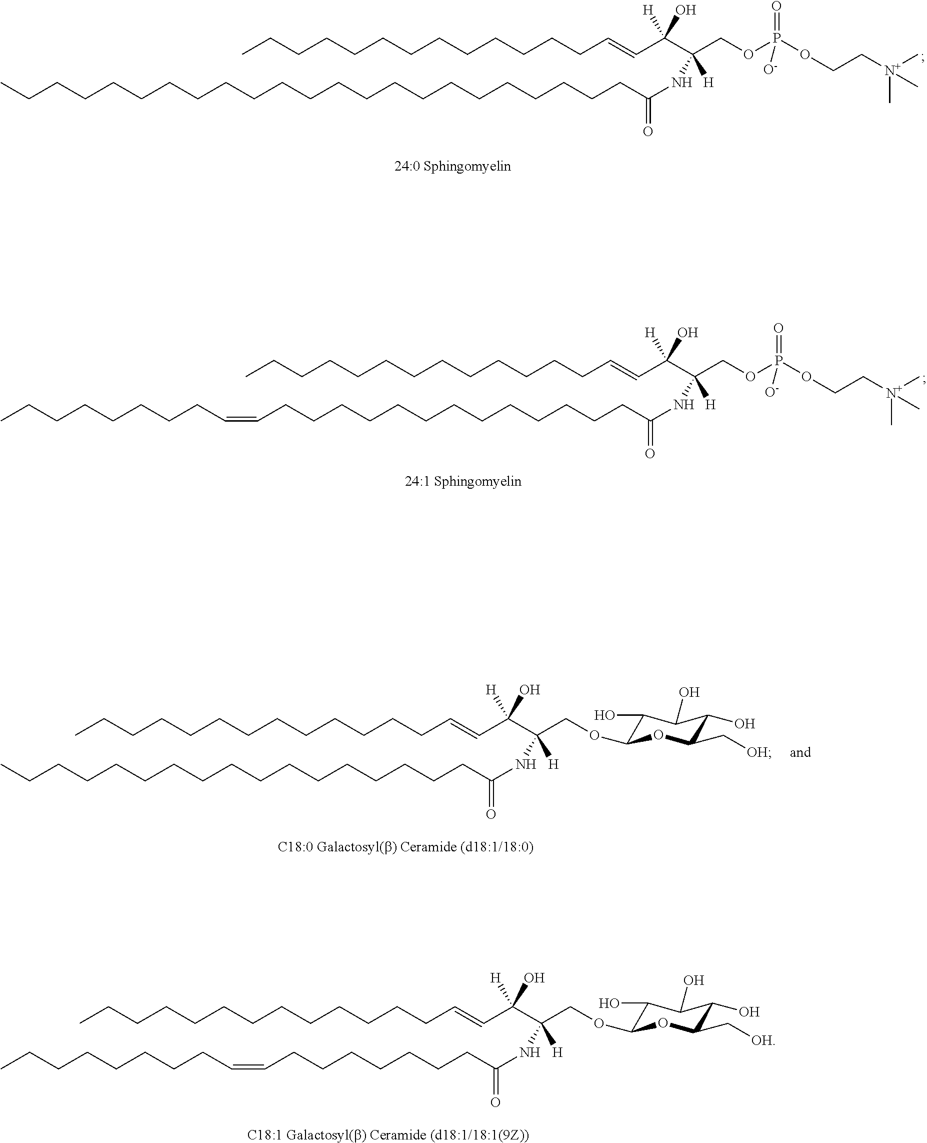

16. The nanobiologic composition of claim 15, wherein the sphingolipid is selected from the group consisting of:

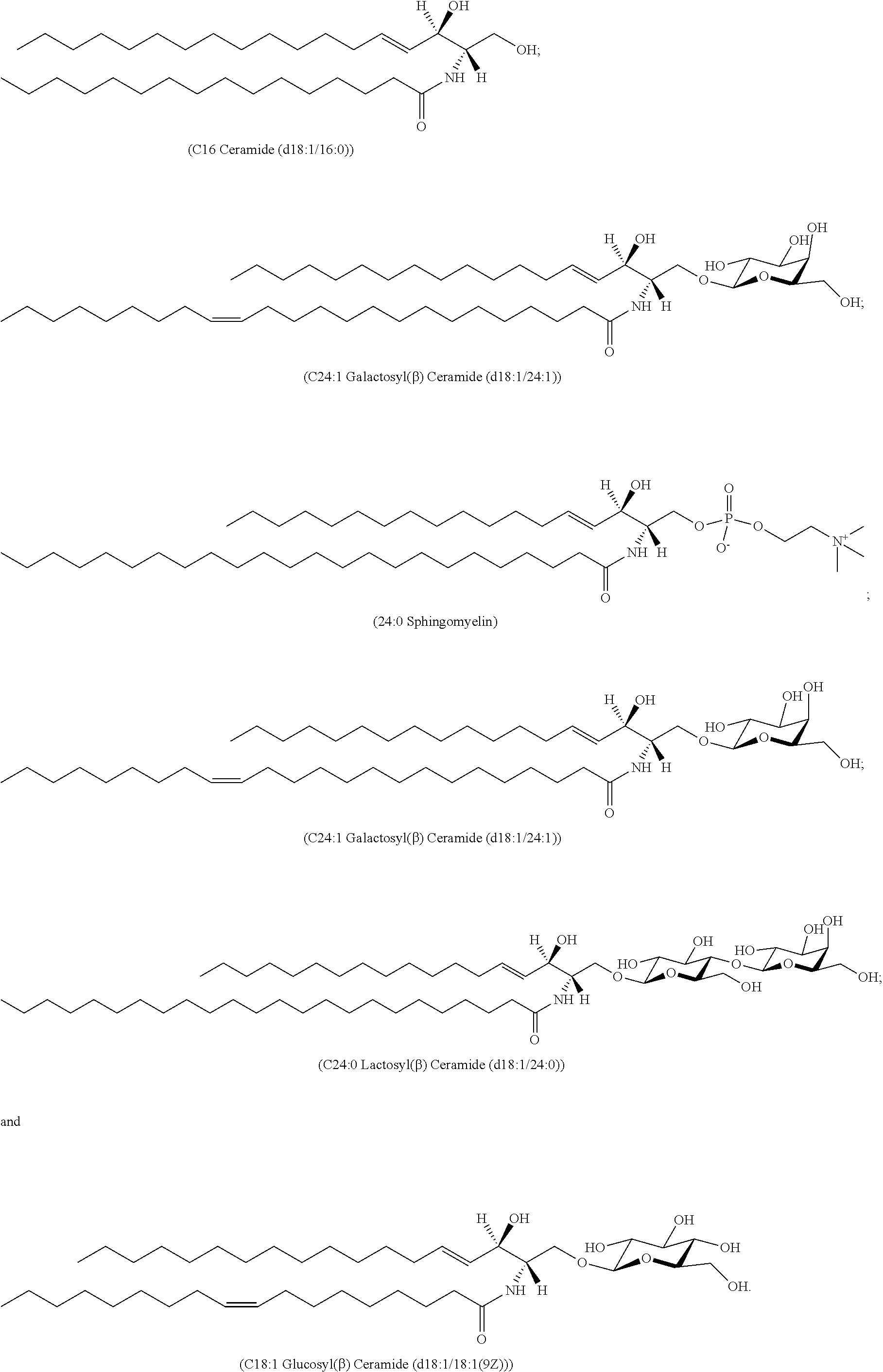

17. The nanobiologic composition of claim 15, wherein the sphingolipid is selected from the group consisting of:

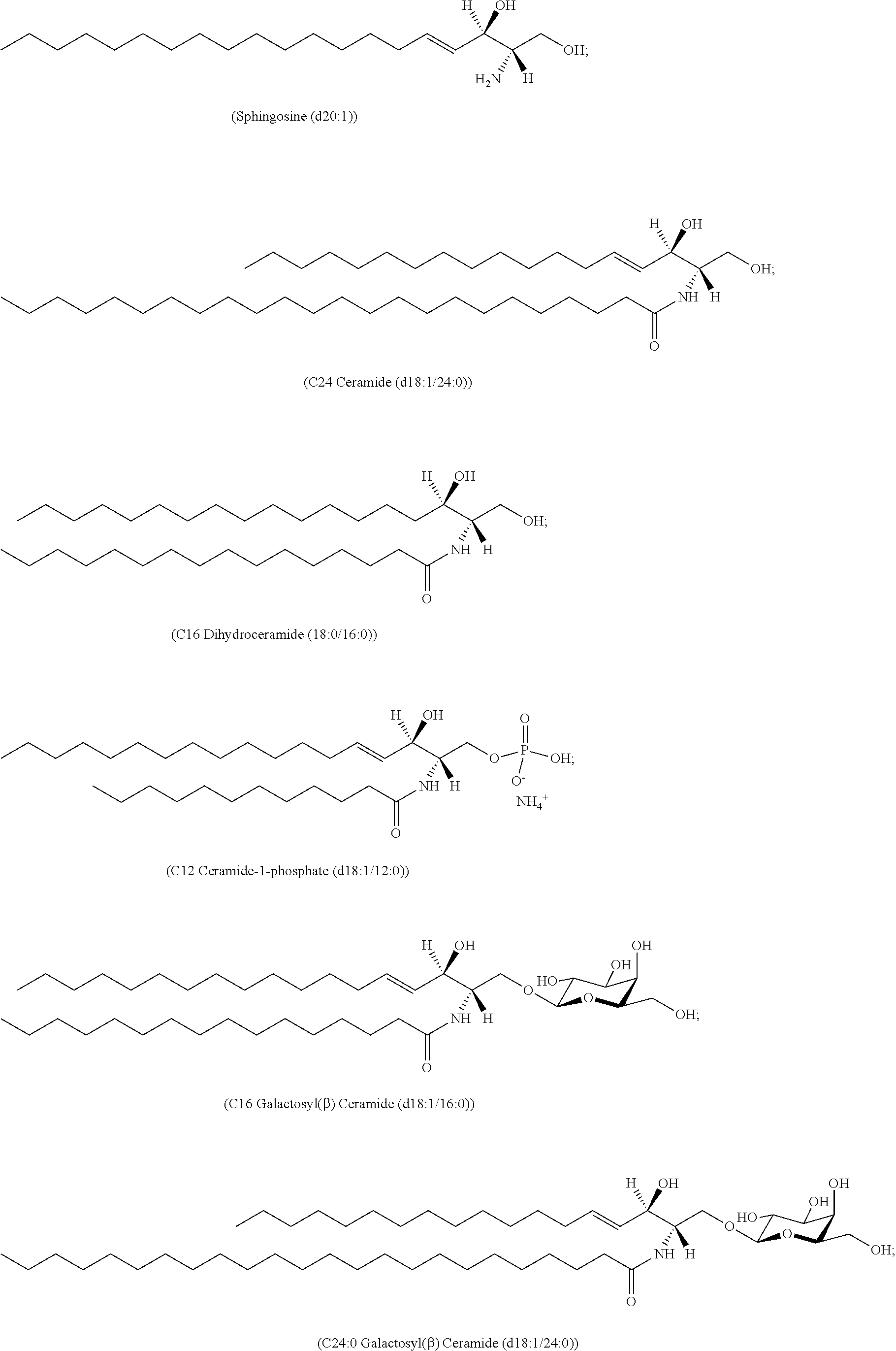

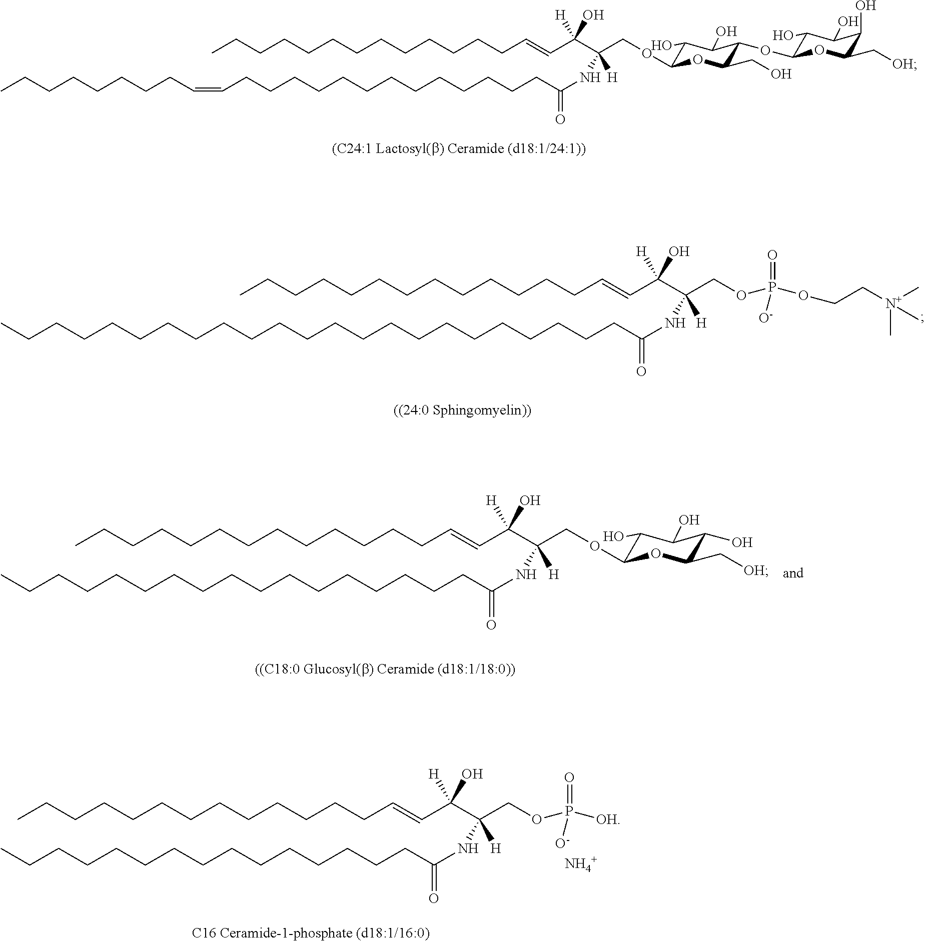

18. The nanobiologic composition of any one of claims 1-17, comprising a phospholipid.

19. The nanobiologic composition of claim 18, wherein the phospholipid is selected from the group consisting of 1,2-dimyristoyl-sn-glycero-3-phosphocholine (DMPC), 1-palmitoyl-2-oleoyl-sn-glycero-3-phosphocholine (POPC), 1,2-dioleoyl-sn-glycero-3-phosphocholine (DOPC) and mixtures thereof.

20. The nanobiologic composition of claim 19, wherein the phospholipid is DMPC.

21. The nanobiologic composition of any one of claims 1-17, comprising a phospholipid and a lysophospholipid.

22. The nanobiologic composition of any one of claims 1-21, wherein the phospholipid is 1,2-dimyristoyl-sn-glycero-3-phosphocholine (DMPC) or 1-palmitoyl-2-oleoyl-sn-glycero-3-phosphocholine (POPC).

23. The nanobiologic composition of claim 21, wherein the lysophospholipid is selected from the group consisting of 1-myristoyl-2-hydroxy-sn-glycero-3-phosphocholine (MHPC), 1-palmitoyl-2-hydroxy-sn-glycero-3-phosphocholine (PHPC), 1-stearoyl-2-hydroxy-sn-glycero-3-phosphocholine (SHPC), and mixtures thereof.

24. The nanobiologic composition of claim 21, wherein the phospholipid is 1-palmitoyl-2-oleoyl-sn-glycero-3-phosphocholine (POPC) and the lysophospholipid is 1-palmitoyl-2-hydroxy-sn-glycero-3-phosphocholine (PHPC).

25. The nanobiologic composition of any one of claims 1-24, wherein the nanobiologic composition comprises human apolipoprotein A-I (apoA-I).

26. The nanobiologic composition of any one of claims 18-25, wherein the sphingolipid is present in about 1-50 mol % of the total lipid composition.

27. The nanobiologic composition of claim 26, wherein the sphingolipid is present in about 10-25 mol % of the total lipid composition.

28. The nanobiologic composition of claim 27, wherein the sphingolipid is present in about 20 mol % of the total lipid composition.

29. The nanobiologic composition of any one of claims 1-28, wherein the cholesterol is present in about 1-30 mol % relative to the phospholipid.

30. The nanobiologic composition of claim 29, wherein the cholesterol is present in about 5-25 mol % relative to the phospholipid.

31. The nanobiologic composition of any one of claims 1-30, wherein the phospholipid, sphingolipid, and cholesterol are present in a molar ratio of about 1:0.05-0.25:0.05-0.25.

32. The nanobiologic composition of any one of claims 1-31, wherein the nanobiologic composition has a PDI of about 0.1 to about 0.3.

33. The nanobiologic composition of any one of claims 1-32, wherein the nanoparticle has a diameter between about 20 nm to about 100 nm.

34. The nanobiologic composition of claim 33, wherein the nanoparticle has a diameter between about 25 nm to about 60 nm.

35. The nanobiologic composition of any one of claims 1-34, wherein the nanoparticle is spherical.

36. The nanobiologic composition of any one of claims 1-35, wherein the nanoparticle is discoidal.

37. The nanobiologic composition of any one of claims 1-36, wherein the nanobiologic composition is suitable for intravenous or intra-arterial administration.

38. A method for treating a cancer in a subject in need thereof, comprising administering to the subject an effective amount of a nanobiologic composition of any one of claims 1-37.

39. The method of claim 38, wherein the cancer is selected from the group consisting of bladder cancer, cancer of the blood vessels, bone cancer, brain cancer, breast cancer, cervical cancer, chest cancer, colon cancer, endometrial cancer, esophageal cancer, eye cancer, head cancer, kidney cancer, liver cancer, cancer of the lymph nodes, lung cancer, mouth cancer, neck cancer, ovarian cancer, pancreatic cancer, prostate cancer, rectal cancer, colorectal cancer, skin cancer, stomach cancer, testicular cancer, throat cancer, thyroid cancer, urothelial cancers, and uterine cancer.

40. The method of claim 39, wherein the cancer is selected from the group consisting of breast cancer, prostate cancer, melanoma, colorectal cancer, lung cancer, pancreatic cancer, and glioblastoma.

41. The method of any one of claims 38-40, wherein the nanobiologic composition is administered intravenously or intra-arterially.

42. The method of any one of claims 38-41, wherein the subject is a human.

43. A method of stimulating a trained immunity response in a subject comprising administering
(i) a nanobiologic composition of any one of claims 1-37; and
(ii) a trained immune promoter.

44. The method of claim 43, wherein the trained immunity promoter is a Dectin-1 receptor agonist, a NOD-2 agonist, or a combination thereof.

45. The method of claim 43, wherein the nanobiologic composition comprises the trained immunity promoter.

46. The method of claim 43, wherein the nanobiologic composition and the trained immunity promoter are administered in separate compositions.

47. The method of claim 46, wherein the nanobiologic composition and trained immunity promoter are administered within up to 1, 2, 3, 4, 5, 6, or 7 days of each other.

48. The method of claim 46 or 47, wherein the routes of administration are the same.

49. The method of claim 48, wherein the route is intravenous,

50. The method of claim 46 or 47, wherein the routes of administration are different.

51. The method of claim 50, wherein the nanobiologic composition is administered intravenously.

52. The method of claim 43, wherein the trained immunity promoter is a Dectin-1 receptor agonist.

53. The method of claim 52, wherein the Dectin-1 receptor agonist is an HKCA (heat-killed Candida albicans).

54. The method of claim 43, wherein the trained immunity promoter is a NOD-2 agonist.

55. The method of claim 54, wherein the NOD2-agonist is a muramyl dipeptide (MDP) a muramyl tri-peptide (MTP) or a derivative or pro-drug thereof.

56. The method of claim 54, wherein NOD2-agonist is a muramyl tripeptide phosphatidylethanolamine.


SEQUENCE LISTING (349 sequences)

SEQ ID NO: 1 - protein, 22 aa, synthetic construct
  REGION 1..22: note = synthetic
PVLDLFRELLNELLEZLKQKLK

SEQ ID NO: 2 - protein, 23 aa, synthetic construct
  REGION 1..23: note = synthetic
GVLDLFRELLNELLEALKQKLKK

SEQ ID NO: 3 - protein, 22 aa, synthetic construct
  REGION 1..22: note = synthetic
PVLDLFRELLNELLEWLKQKLK

SEQ ID NO: 4 - protein, 22 aa, synthetic construct
  REGION 1..22: note = synthetic
PVLDLFRELLNELLEALKQKLK

SEQ ID NO: 5 - protein, 23 aa, synthetic construct
  REGION 1..23: note = synthetic
PVLDLFRELLNELLEALKQKLKK

SEQ ID NO: 6 - protein, 22 aa, synthetic construct
  VARIANT 1..22: note = Xaa = Aib
PVLDLFRELLNEXLEALKQKLK

SEQ ID NO: 7 - protein, 22 aa, synthetic construct
  REGION 1..22: note = synthetic
PVLDLFKELLNELLEALKQKLK

SEQ ID NO: 8 - protein, 22 aa, synthetic construct
  REGION 1..22: note = synthetic
PVLDLFRELLNEGLEALKQKLK

SEQ ID NO: 9 - protein, 22 aa, synthetic construct
  REGION 1..22: note = synthetic
PVLDLFRELGNELLEALKQKLK

SEQ ID NO: 10 - protein, 22 aa, synthetic construct
  REGION 1..22: note = synthetic
PVLDLFRELLNELLEAZKQKLK

SEQ ID NO: 11 - protein, 22 aa, synthetic construct
  REGION 1..22: note = synthetic
PVLDLFKELLQELLEALKQKLK

SEQ ID NO: 12 - protein, 22 aa, synthetic construct
  REGION 1..22: note = synthetic
PVLDLFRELLNELLEAGKQKLK

SEQ ID NO: 13 - protein, 22 aa, synthetic construct
  REGION 1..22: note = synthetic
GVLDLFRELLNEGLEALKQKLK

SEQ ID NO: 14 - protein, 22 aa, synthetic construct
  VARIANT 1..22: note = Xaa = Orn
PVLDLFRELLNELLEALXQXLX

SEQ ID NO: 15 - protein, 22 aa, synthetic construct
  REGION 1..22: note = synthetic
PVLDLFRELWNELLEALKQKLK

SEQ ID NO: 16 - protein, 22 aa, synthetic construct
  REGION 1..22: note = synthetic
PVLDLLRELLNELLEALKQKLK

SEQ ID NO: 17 - protein, 22 aa, synthetic construct
  REGION 1..22: note = synthetic
PVLELFKELLQELLEALKQKLK

SEQ ID NO: 18 - protein, 22 aa, synthetic construct
  REGION 1..22: note = synthetic
GVLDLFRELLNELLEALKQKLK

SEQ ID NO: 19 - protein, 22 aa, synthetic construct
  REGION 1..22: note = synthetic
PVLDLFRELLNEGLEALKQKLK

SEQ ID NO: 20 - protein, 22 aa, synthetic construct
  REGION 1..22: note = synthetic
PVLDLFREGLNELLEALKQKLK

SEQ ID NO: 21 - protein, 22 aa, synthetic construct
  REGION 1..22: note = synthetic
PVLDLFRELLNELLEALKQKLK

SEQ ID NO: 22 - protein, 22 aa, synthetic construct
  REGION 1..22: note = synthetic
PVLDLFRELLNELLEGLKQKLK

SEQ ID NO: 23 - protein, 22 aa, synthetic construct
  REGION 1..22: note = synthetic
PLLELFKELLQELLEALKQKLK

SEQ ID NO: 24 - protein, 22 aa, synthetic construct
  REGION 1..22: note = synthetic
PVLDLFRELLNELLEALQKKLK

SEQ ID NO: 25 - protein, 22 aa, synthetic construct
  VARIANT 1..22: note = Xaa = Aib
PVLDFFRELLNEXLEALKQKLK

SEQ ID NO: 26 - protein, 22 aa, synthetic construct
  REGION 1..22: note = synthetic
PVLDLFRELLNELLELLKQKLK

SEQ ID NO: 27 - protein, 22 aa, synthetic construct
  REGION 1..22: note = synthetic
PVLDLFRELLNELZEALKQKLK

SEQ ID NO: 28 - protein, 22 aa, synthetic construct
  REGION 1..22: note = synthetic
PVLDLFRELLNELWEALKQKLK

SEQ ID NO: 29 - protein, 22 aa, synthetic construct
  REGION 1..22: note = synthetic
AVLDLFRELLNELLEALKQKLK

SEQ ID NO: 30 - protein, 22 aa, synthetic construct
  REGION 1..22: note = synthetic
PVLDLPRELLNELLEALKQKLK

SEQ ID NO: 31 - protein, 22 aa, synthetic construct
  VARIANT 1..22: note = Xaa = Aib
PVLDLFLELLNEXLEALKQKLK

SEQ ID NO: 32 - protein, 22 aa, synthetic construct
  VARIANT 1..22: note = Xaa = Aib
XVLDLFRELLNELLEALKQKLK

SEQ ID NO: 33 - protein, 22 aa, synthetic construct
  REGION 1..22: note = synthetic
PVLDLFREKLNELLEALKQKLK

SEQ ID NO: 34 - protein, 22 aa, synthetic construct
  REGION 1..22: note = synthetic
PVLDZFRELLNELLEALKQKLK

SEQ ID NO: 35 - protein, 22 aa, synthetic construct
  REGION 1..22: note = synthetic
PVLDWFRELLNELLEALKQKLK

SEQ ID NO: 36 - protein, 22 aa, synthetic construct
  REGION 1..22: note = synthetic
PLLELLKELLQELLEALKQKLK

SEQ ID NO: 37 - protein, 22 aa, synthetic construct
  REGION 1..22: note = synthetic
PVLDLFREWLNELLEALKQKLK

SEQ ID NO: 38 - protein, 22 aa, synthetic construct
  VARIANT 1..22: note = Xaa = Aib
PVLDLFRELLNEXLEAWKQKLK

SEQ ID NO: 39 - protein, 22 aa, synthetic construct
  REGION 1..22: note = synthetic
PVLDLFRELLEELLKALKKKLK

SEQ ID NO: 40 - protein, 22 aa, synthetic construct
  REGION 1..22: note = synthetic
PVLDLFNELLRELLEALQKKLK

SEQ ID NO: 41 - protein, 22 aa, synthetic construct
  VARIANT 1..22: note = Xaa = Aib
PVLDLWRELLNEXLEALKQKLK

SEQ ID NO: 42 - protein, 22 aa, synthetic construct
  VARIANT 1..22: note = Xaa = Aib
PVLDEFREKLNEXWEALKQKLK

SEQ ID NO: 43 - protein, 22 aa, synthetic construct
  VARIANT 1..22: note = Xaa = Aib
PVLDEFREKLWEXLEALKQKLK

SEQ ID NO: 44 - protein, 22 aa, synthetic construct
  VARIANT 1..22: note = Xaa = Aib
PVLDEFREKLNEXLEALKQKLK

SEQ ID NO: 45 - protein, 22 aa, synthetic construct
  VARIANT 1..22: note = Xaa = Aib
PVLDEFREKLNEXLEALKQKLK

SEQ ID NO: 46 - protein, 22 aa, synthetic construct
  VARIANT 1..22: note = Xaa = Aib
PVLDLFREKLNEXLEALKQKLK

SEQ ID NO: 47 - protein, 21 aa, synthetic construct
  REGION 1..21: note = synthetic
VLDLFRELLNEGLEALKQKLK

SEQ ID NO: 48 - protein, 22 aa, synthetic construct
  REGION 1..22: note = synthetic
PVLDLFRELLNELLEALKQKLK

SEQ ID NO: 49 - protein, 22 aa, synthetic construct
  REGION 1..22: note = synthetic
PVLDLFRNLLEKLLEALEQKLK

SEQ ID NO: 50 - protein, 22 aa, synthetic construct
  VARIANT 1..22: note = Xaa = Aib
PVLDLFRELLWEXLEALKQKLK

SEQ ID NO: 51 - protein, 22 aa, synthetic construct
  VARIANT 1..22: note = Xaa = Aib
PVLDLFWELLNEXLEALKQKLK

SEQ ID NO: 52 - protein, 22 aa, synthetic construct
  VARIANT 1..22: note = Xaa = Aib
PVWDEFREKLNEXLEALKQKLK

SEQ ID NO: 53 - protein, 22 aa, synthetic construct
  REGION 1..22: note = synthetic
VVLDLFRELLNELLEALKQKLK

SEQ ID NO: 54 - protein, 22 aa, synthetic construct
  REGION 1..22: note = synthetic
PVLDLFRELLNEWLEALKQKLK

SEQ ID NO: 55 - protein, 19 aa, synthetic construct
  REGION 1..19: note = synthetic
PLFRELLNELLEALKQKLK

SEQ ID NO: 56 - protein, 22 aa, synthetic construct
  REGION 1..22: note = synthetic
PVLDLFRELLNELLEALKQKKK

SEQ ID NO: 57 - protein, 22 aa, synthetic construct
  REGION 1..22: note = synthetic
PVLDLFRNLLEELLKALEQKLK

SEQ ID NO: 58 - protein, 21 aa, synthetic construct
  VARIANT 1..21: note = Xaa = Aib
PVLDEFREKLNEXLEALKQKL

SEQ ID NO: 59 - protein, 22 aa, synthetic construct
  REGION 1..22: note = synthetic
LVLDLFRELLNELLEALKQKLK

SEQ ID NO: 60 - protein, 19 aa, synthetic construct
  REGION 1..19: note = synthetic
PVLDLFRELLNELLEALKQ

SEQ ID NO: 61 - protein, 22 aa, synthetic construct
  VARIANT 1..22: note = Xaa = Aib
PVLDEFRWKLNEXLEALKQKLK

SEQ ID NO: 62 - protein, 22 aa, synthetic construct
  VARIANT 1..22: note = Xaa = Aib
PVLDEWREKLNEXLEALKQKLK

SEQ ID NO: 63 - protein, 22 aa, synthetic construct
  VARIANT 1..22: note = Xaa = Aib
PVLDFFREKLNEXLEALKQKLK

SEQ ID NO: 64 - protein, 22 aa, synthetic construct
  VARIANT 1..22: note = Xaa = Aib
PWLDEFREKLNEXLEALKQKLK

SEQ ID NO: 65 - protein, 21 aa, synthetic construct
  VARIANT 1..21: note = Xaa = Aib
VLDEFREKLNEXLEALKQKLK

SEQ ID NO: 66 - protein, 22 aa, synthetic construct
  REGION 1..22: note = synthetic
PVLDLFRNLLEELLEALQKKLK

SEQ ID NO: 67 - protein, 21 aa, synthetic construct
  REGION 1..21: note = synthetic
VLDLFRELLNELLEALKQKLK

SEQ ID NO: 68 - protein, 22 aa, synthetic construct
  VARIANT 1..22: note = Xaa = Aib
PVLDEFRELLKEXLEALKQKLK

SEQ ID NO: 69 - protein, 22 aa, synthetic construct
  VARIANT 1..22: note = Xaa = Aib
PVLDEFRKKLNEXLEALKQKLK

SEQ ID NO: 70 - protein, 22 aa, synthetic construct
  VARIANT 1..22: note = Xaa = Aib
PVLDEFRELLYEXLEALKQKLK

SEQ ID NO: 71 - protein, 22 aa, synthetic construct
  VARIANT 1..22: note = Xaa = Aib
PVLDEFREKLNELXEALKQKLK

SEQ ID NO: 72 - protein, 22 aa, synthetic construct
  VARIANT 1..22: note = Xaa = Aib
PVLDLFRELLNEXLWALKQKLK

SEQ ID NO: 73 - protein, 22 aa, synthetic construct
  VARIANT 1..22: note = Xaa = Aib
PVLDEFWEKLNEXLEALKQKLK

SEQ ID NO: 74 - protein, 22 aa, synthetic construct
  VARIANT 1..22: note = Xaa = Aib
PVLDKFREKLNEXLEALKQKLK

SEQ ID NO: 75 - protein, 22 aa, synthetic construct
  REGION 1..22: note = synthetic
PVLDEFREKLNEELEALKQKLK

SEQ ID NO: 76 - protein, 22 aa, synthetic construct
  VARIANT 1..22: note = Xaa = Aib
PVLDEFRELLFEXLEALKQKLK

SEQ ID NO: 77 - protein, 22 aa, synthetic construct
  VARIANT 1..22: note = Xaa = Aib
PVLDEFREKLNKXLEALKQKLK

SEQ ID NO: 78 - protein, 22 aa, synthetic construct
PVLDEFRDKLNEXLEALKQKLK

SEQ ID NO: 79 - protein, 22 aa, synthetic construct
PVLDEFRELLNELLEALKQKLK

SEQ ID NO: 80 - protein, 22 aa, synthetic construct
PVLDLFERLLNELLEALQKKLK

SEQ ID NO: 81 - protein, 22 aa, synthetic construct
  VARIANT 1..22: note = Xaa = Aib
PVLDEFREKLNWXLEALKQKLK

SEQ ID NO: 82 - protein, 20 aa, synthetic construct
  VARIANT 1..20: note = Xaa = Aib
LDEFREKLNEXLEALKQKLK

SEQ ID NO: 83 - protein, 22 aa, synthetic construct
  REGION 1..22: note = synthetic
  REGION 1..22: note = misc_feature - Xaa = Aib
  SITE 13: note = misc_feature - Xaa can be any naturally occurring amino acid
PVLDEFREKLNEXLEALWQKLK

SEQ ID NO: 84 - protein, 22 aa, synthetic construct
  REGION 1..22: note = synthetic
PVLDEFREKLNELLEALKQKLK

SEQ ID NO: 85 - protein, 21 aa, synthetic construct
  REGION 1..21: note = synthetic
PLDLFRELLNELLEALKQKLK

SEQ ID NO: 86 - protein, 22 aa, synthetic construct
  REGION 1..22: note = synthetic
PVLELFERLLDELLNALQKKLK

SEQ ID NO: 87 - protein, 22 aa, synthetic construct
  REGION 1..22: note = synthetic
PLLELLKELLQELLEALKQKLK

SEQ ID NO: 88 - protein, 22 aa, synthetic construct
  VARIANT 1..22: note = Xaa = Aib
PVLDKFRELLNEXLEALKQKLK

SEQ ID NO: 89 - protein, 22 aa, synthetic construct
  VARIANT 1..22: note = Xaa = Aib
PVLDEFREKLNEXLWALKQKLK

SEQ ID NO: 90 - protein, 19 aa, synthetic construct
  VARIANT 1..19: note = Xaa = Aib
DEFREKLNEXLEALKQKLK

SEQ ID NO: 91 - protein, 22 aa, synthetic construct
  VARIANT 1..22: note = Xaa = Aib
PVLDEFRELLNEXLEALKQKLK

SEQ ID NO: 92 - protein, 22 aa, synthetic construct
  VARIANT 1..22: note = Xaa = Aib
PVLDEFRELYNEXLEALKQKLK

SEQ ID NO: 93 - protein, 22 aa, synthetic construct
  VARIANT 1..22: note = Xaa = Aib
PVLDEFREKLNEXLKALKQKLK

SEQ ID NO: 94 - protein, 22 aa, synthetic construct
  REGION 1..22: note = synthetic
PVLDEFREKLNEALEALKQKLK

SEQ ID NO: 95 - protein, 22 aa, synthetic construct
  VARIANT 1..22: note = Xaa = Aib
PVLDLFRELLNLXLEALKQKLK

SEQ ID NO: 96 - protein, 22 aa, synthetic construct
  VARIANT 1..22: note = Xaa = Aib
PVLDLFRELLNEXLEALKQKLK

SEQ ID NO: 97 - protein, 15 aa, synthetic construct
  REGION 1..15: note = synthetic
PVLDLFRELLNELLE

SEQ ID NO: 98 - protein, 22 aa, synthetic construct
  REGION 1..22: note = synthetic
PVLDLFRELLNEELEALKQKLK

SEQ ID NO: 99 - protein, 22 aa, synthetic construct
  REGION 1..22: note = synthetic
KLKQKLAELLENLLERFLDLVP

SEQ ID NO: 100 - protein, 22 aa, synthetic construct
  REGION 1..22: note = synthetic
PVLDLFRELLNELLEALKQKLK

SEQ ID NO: 101 - protein, 22 aa, synthetic construct
  VARIANT 1..22: note = Xaa = Aib
PVLDLFRELLNWXLEALKQKLK

SEQ ID NO: 102 - protein, 22 aa, synthetic construct
  VARIANT 1..22: note = Xaa = Aib
PVLDLFRELLNLXLEALKEKLK

SEQ ID NO: 103 - protein, 22 aa, synthetic construct
  REGION 1..22: note = synthetic
PVLDEFRELLNEELEALKQKLK

SEQ ID NO: 104 - protein, 15 aa, synthetic construct
  REGION 1..15: note = synthetic
PLLNELLEALKQKLK

SEQ ID NO: 105 - protein, 22 aa, synthetic construct
  REGION 1..22: note = synthetic
PAADAFREAANEAAEAAKQKAK

SEQ ID NO: 106 - protein, 22 aa, synthetic construct
  REGION 1..22: note = synthetic
PVLDLFREKLNEELEALKQKLK

SEQ ID NO: 107 - protein, 22 aa, synthetic construct
  REGION 1..22: note = synthetic
KLKQKLAELLENLLERFLDLVP

SEQ ID NO: 108 - protein, 22 aa, synthetic construct
  VARIANT 1..22: note = Xaa = Aib
PVLDLFRWLLNEXLEALKQKLK

SEQ ID NO: 109 - protein, 22 aa, synthetic construct
  REGION 1..22: note = synthetic
PVLDEFREKLNERLEALKQKLK

SEQ ID NO: 110 - protein, 22 aa, synthetic construct
  VARIANT 1..22: note = Xaa = Aib
PVLDEFREKLNEXXEALKQKLK

SEQ ID NO: 111 - protein, 22 aa, synthetic construct
  VARIANT 1..22: note = Xaa = Aib
PVLDEFREKLWEXWEALKQKLK

SEQ ID NO: 112 - protein, 22 aa, synthetic construct
  VARIANT 1..22: note = Xaa = Aib
PVLDEFREKLNEXSEALKQKLK

SEQ ID NO: 113 - protein, 22 aa, synthetic construct
  REGION 1..22: note = synthetic
PVLDEFREKLNEPLEALKQKLK

SEQ ID NO: 114 - protein, 22 aa, synthetic construct
  VARIANT 1..22: note = Xaa = Aib
PVLDEFREKLNEXMEALKQKLK

SEQ ID NO: 115 - protein, 22 aa, synthetic construct
  VARIANT 1..22: note = Xaa = Aib
PKLDEFREKLNEXLEALKQKLK

SEQ ID NO: 116 - protein, 22 aa, synthetic construct
  VARIANT 1..22: note = Xaa = Aib
PHLDEFREKLNEXLEALKQKLK

SEQ ID NO: 117 - protein, 22 aa, synthetic construct
  VARIANT 1..22: note = Xaa = Aib
PELDEFREKLNEXLEALKQKLK

SEQ ID NO: 118 - protein, 22 aa, synthetic construct
  VARIANT 1..22: note = Xaa = Aib
PVLDEFREKLNEXLEALEQKLK

SEQ ID NO: 119 - protein, 22 aa, synthetic construct
  VARIANT 1..22: note = Xaa = Aib
PVLDEFREKLNEELEAXKQKLK

SEQ ID NO: 120 - protein, 22 aa, synthetic construct
  VARIANT 1..22: note = Xaa = Aib
PVLDEFREKLNEELEXLKQKLK

SEQ ID NO: 121 - protein, 22 aa, synthetic construct
  REGION 1..22: note = synthetic
PVLDEFREKLNEELEALWQKLK

SEQ ID NO: 122 - protein, 22 aa, synthetic construct
  REGION 1..22: note = synthetic
PVLDEFREKLNEELEWLKQKLK

SEQ ID NO: 123 - protein, 22 aa, synthetic construct
  REGION 1..22: note = synthetic
QVLDLFRELLNELLEALKQKLK

SEQ ID NO: 124 - protein, 22 aa, synthetic construct
  VARIANT 1..22: note = Xaa = Orn
PVLDLFXELLNELLEALXQXLX

SEQ ID NO: 125 - protein, 22 aa, synthetic construct
  REGION 1..22: note = synthetic
NVLDLFRELLNELLEALKQKLK

SEQ ID NO: 126 - protein, 22 aa, synthetic construct
  REGION 1..22: note = synthetic
PVLDLFRELLNELGEALKQKLK

SEQ ID NO: 127 - protein, 22 aa, synthetic construct
  REGION 1..22: note = synthetic
PVLDLFRELLNELLELLKQKLK

SEQ ID NO: 128 - protein, 22 aa, synthetic construct
  REGION 1..22: note = synthetic
PVLDLFRELLNELLEFLKQKLK

SEQ ID NO: 129 - protein, 22 aa, synthetic construct
  REGION 1..22: note = synthetic
PVLELFNDLLRELLEALQKKLK

SEQ ID NO: 130 - protein, 22 aa, synthetic construct
  REGION 1..22: note = synthetic
PVLELFNDLLRELLEALKQKLK

SEQ ID NO: 131 - protein, 22 aa, synthetic construct
  REGION 1..22: note = synthetic
PVLELFKELLNELLDALRQKLK

SEQ ID NO: 132 - protein, 22 aa, synthetic construct
  REGION 1..22: note = synthetic
PVLDLFRELLENLLEALQKKLK

SEQ ID NO: 133 - protein, 22 aa, synthetic construct
  REGION 1..22: note = synthetic
PVLELFERLLEDLLQALNKKLK

SEQ ID NO: 134 - protein, 22 aa, synthetic construct
  VARIANT 1..22: note = Xaa = Orn
PVLELFERLLEDLLKALNXKLK

SEQ ID NO: 135 - protein, 22 aa, synthetic construct
  REGION 1..22: note = synthetic
DVLDLFRELLNELLEALKQKLK

SEQ ID NO: 136 - protein, 22 aa, synthetic construct
  REGION 1..22: note = synthetic
PALELFKDLLQELLEALKQKLK

SEQ ID NO: 137 - protein, 22 aa, synthetic construct
  REGION 1..22: note = synthetic
PVLDLFRELLNEGLEAZKQKLK

SEQ ID NO: 138 - protein, 22 aa, synthetic construct
  REGION 1..22: note = synthetic
PVLDLFRELLNEGLEWLKQKLK

SEQ ID NO: 139 - protein, 22 aa, synthetic construct
  REGION 1..22: note = synthetic
PVLDLFRELWNEGLEALKQKLK

SEQ ID NO: 140 - protein, 22 aa, synthetic construct
  VARIANT 1..22: note = Xaa = Orn
PVLDLFRELLNEGLEALXQXLX

SEQ ID NO: 141 - protein, 22 aa, synthetic construct
  REGION 1..22: note = synthetic
PVLDFFRELLNEGLEALKQKLK

SEQ ID NO: 142 - protein, 22 aa, synthetic construct
  REGION 1..22: note = synthetic
PVLELFRELLNEGLEALKQKLK

SEQ ID NO: 143 - protein, 22 aa, synthetic construct
  REGION 1..22: note = synthetic
PVLDLFRELLNEGLEALKQKLK

SEQ ID NO: 144 - protein, 22 aa, synthetic construct
  REGION 1..22: note = synthetic
PVLELFENLLERLLDALQKKLK

SEQ ID NO: 145 - protein, 22 aa, synthetic construct
  REGION 1..22: note = synthetic
GVLELFENLLERLLDALQKKLK

SEQ ID NO: 146 - protein, 22 aa, synthetic construct
  REGION 1..22: note = synthetic
PVLELFENLLERLLDALQKKLK

SEQ ID NO: 147 - protein, 22 aa, synthetic construct
  REGION 1..22: note = synthetic
PVLELFENLLERLFDALQKKLK

SEQ ID NO: 148 - protein, 22 aa, synthetic construct
  REGION 1..22: note = synthetic
PVLELFENLLERLGDALQKKLK

SEQ ID NO: 149 - protein, 22 aa, synthetic construct
  REGION 1..22: note = synthetic
PVLELFENLWERLLDALQKKLK

SEQ ID NO: 150 - protein, 22 aa, synthetic construct
  REGION 1..22: note = synthetic
PLLELFENLLERLLDALQKKLK

SEQ ID NO: 151 - protein, 22 aa, synthetic construct
  REGION 1..22: note = synthetic
PVLELFENLGERLLDALQKKLK

SEQ ID NO: 152 - protein, 22 aa, synthetic construct
  REGION 1..22: note = synthetic
PVFELFENLLERLLDALQKKLK

SEQ ID NO: 153 - protein, 22 aa, synthetic construct
  REGION 1..22: note = synthetic
AVLELFENLLERLLDALQKKLK

SEQ ID NO: 154 - protein, 22 aa, synthetic construct
  REGION 1..22: note = synthetic
PVLELFENLLERGLDALQKKLK

SEQ ID NO: 155 - protein, 22 aa, synthetic construct
  REGION 1..22: note = synthetic
PVLELFLNLWERLLDALQKKLK

SEQ ID NO: 156 - protein, 22 aa, synthetic construct
  REGION 1..22: note = synthetic
PVLELFLNLLERLLDALQKKLK

SEQ ID NO: 157 - protein, 22 aa, synthetic construct
  REGION 1..22: note = synthetic
PVLEFFENLLERLLDALQKKLK

SEQ ID NO: 158 - protein, 22 aa, synthetic construct
  REGION 1..22: note = synthetic
PVLELFLNLLERLLDWLQKKLK

SEQ ID NO: 159 - protein, 22 aa, synthetic construct
  REGION 1..22: note = synthetic
PVLDLFENLLERLLDALQKKLK

SEQ ID NO: 160 - protein, 22 aa, synthetic construct
  REGION 1..22: note = synthetic
PVLELFENLLERLLDWLQKKLK

SEQ ID NO: 161 - protein, 22 aa, synthetic construct
  REGION 1..22: note = synthetic
PVLELFENLLERLLEALQKKLK

SEQ ID NO: 162 - protein, 22 aa, synthetic construct
  REGION 1..22: note = synthetic
PVLELFENWLERLLDALQKKLK

SEQ ID NO: 163 - protein, 22 aa, synthetic construct
  REGION 1..22: note = synthetic
PVLELFENLLERLWDALQKKLK

SEQ ID NO: 164 - protein, 22 aa, synthetic construct
  REGION 1..22: note = synthetic
PVLELFENLLERLLDAWQKKLK

SEQ ID NO: 165 - protein, 22 aa, synthetic construct
  REGION 1..22: note = synthetic
PVLELFENLLERLLDLLQKKLK

SEQ ID NO: 166 - protein, 22 aa, synthetic construct
  REGION 1..22: note = synthetic
PVLELFLNLLEKLLDALQKKLK

SEQ ID NO: 167 - protein, 22 aa, synthetic construct
  REGION 1..22: note = synthetic
PVLELFENGLERLLDALQKKLK

SEQ ID NO: 168 - protein, 22 aa, synthetic construct
  REGION 1..22: note = synthetic
PVLELFEQLLEKLLDALQKKLK

SEQ ID NO: 169 - protein, 22 aa, synthetic construct
  REGION 1..22: note = synthetic
PVLELFENLLEKLLDALQKKLK

SEQ ID NO: 170 - protein, 22 aa, synthetic construct
  VARIANT 1..22: note = Xaa = Orn
PVLELFENLLEXLLDALQXXLX

SEQ ID NO: 171 - protein, 22 aa, synthetic construct
  REGION 1..22: note = synthetic
PVLELFENLLEKLLDLLQKKLK

SEQ ID NO: 172 - protein, 22 aa, synthetic construct
  REGION 1..22: note = synthetic
PVLELFLNLLERLGDALQKKLK

SEQ ID NO: 173 - protein, 22 aa, synthetic construct
  REGION 1..22: note = synthetic
PVLDLFDNLLDRLLDLLNKKLK

SEQ ID NO: 174 - protein, 22 aa, synthetic construct
  REGION 1..22: note = synthetic
PVLELFENLLERLLDALQKKLK

SEQ ID NO: 175 - protein, 22 aa, synthetic construct
  REGION 1..22: note = synthetic
PVLELFENLLERLLELLNKKLK

SEQ ID NO: 176 - protein, 22 aa, synthetic construct
  REGION 1..22: note = synthetic
PVLELWENLLERLLDALQKKLK

SEQ ID NO: 177 - protein, 22 aa, synthetic construct
  REGION 1..22: note = synthetic
GVLELFLNLLERLLDALQKKLK

SEQ ID NO: 178 - protein, 22 aa, synthetic construct
  REGION 1..22: note = synthetic
PVLELFDNLLEKLLEALQKKLR

SEQ ID NO: 179 - protein, 22 aa, synthetic construct
  REGION 1..22: note = synthetic
PVLELFDNLLERLLDALQKKLK

SEQ ID NO: 180 - protein, 22 aa, synthetic construct
  REGION 1..22: note = synthetic
PVLELFDNLLDKLLDALQKKLR

SEQ ID NO: 181 - protein, 22 aa, synthetic construct
  REGION 1..22: note = synthetic
PVLELFENLLERWLDALQKKLK

SEQ ID NO: 182 - protein, 22 aa, synthetic construct
  REGION 1..22: note = synthetic
PVLELFENLLEKLLEALQKKLK

SEQ ID NO: 183 - protein, 22 aa, synthetic construct
  REGION 1..22: note = synthetic
PLLELFENLLEKLLDALQKKLK

SEQ ID NO: 184 - protein, 22 aa, synthetic construct
  REGION 1..22: note = synthetic
PVLELFLNLLERLLDAWQKKLK

SEQ ID NO: 185 - protein, 22 aa, synthetic construct
  VARIANT 1..22: note = Xaa = Orn
PVLELFENLLERLLDALQXXLX

SEQ ID NO: 186 - protein, 22 aa, synthetic construct
  REGION 1..22: note = synthetic
PVLELFEQLLERLLDALQKKLK

SEQ ID NO: 187 - protein, 22 aa, synthetic construct
  REGION 1..22: note = synthetic
PVLELFENLLERLLDALNKKLK

SEQ ID NO: 188 - protein, 22 aa, synthetic construct
  REGION 1..22: note = synthetic
PVLELFENLLDRLLDALQKKLK

SEQ ID NO: 189 - protein, 22 aa, synthetic construct
  REGION 1..22: note = synthetic
DVLELFENLLERLLDALQKKLK

SEQ ID NO: 190 - protein, 22 aa, synthetic construct
  REGION 1..22: note = synthetic
PVLEFWDNLLDKLLDALQKKLR

SEQ ID NO: 191 - protein, 18 aa, synthetic construct
  REGION 1..18: note = synthetic
PVLDLLRELLEELKQKLK

SEQ ID NO: 192 - protein, 18 aa, synthetic construct
  REGION 1..18: note = synthetic
PVLDLFKELLEELKQKLK

SEQ ID NO: 193 - protein, 18 aa, synthetic construct
  REGION 1..18: note = synthetic
PVLDLFRELLEELKQKLK

SEQ ID NO: 194 - protein, 18 aa, synthetic construct
  REGION 1..18: note = synthetic
PVLELFRELLEELKQKLK

SEQ ID NO: 195 - protein, 18 aa, synthetic construct
  REGION 1..18: note = synthetic
PVLELFKELLEELKQKLK

SEQ ID NO: 196 - protein, 18 aa, synthetic construct
  REGION 1..18: note = synthetic
PVLDLFRELLEELKNKLK

SEQ ID NO: 197 - protein, 18 aa, synthetic construct
  REGION 1..18: note = synthetic
PLLDLFRELLEELKQKLK

SEQ ID NO: 198 - protein, 18 aa, synthetic construct
  REGION 1..18: note = synthetic
GVLDLFRELLEELKQKLK

SEQ ID NO: 199 - protein, 18 aa, synthetic construct
  REGION 1..18: note = synthetic
PVLDLFRELWEELKQKLK

SEQ ID NO: 200 - protein, 18 aa, synthetic construct
  REGION 1..18: note = synthetic
NVLDLFRELLEELKQKLK

SEQ ID NO: 201 - protein, 18 aa, synthetic construct
  REGION 1..18: note = synthetic
PLLDLFKELLEELKQKLK

SEQ ID NO: 202 - protein, 18 aa, synthetic construct
  REGION 1..18: note = synthetic
PALELFKDLLEELRQKLR

SEQ ID NO: 203 - protein, 18 aa, synthetic construct
  REGION 1..18: note = synthetic
AVLDLFRELLEELKQKLK

SEQ ID NO: 204 - protein, 18 aa, synthetic construct
  REGION 1..18: note = synthetic
PVLDFFRELLEELKQKLK

SEQ ID NO: 205 - protein, 18 aa, synthetic construct
  REGION 1..18: note = synthetic
PVLDLFREWLEELKQKLK

SEQ ID NO: 206 - protein, 18 aa, synthetic construct
  REGION 1..18: note = synthetic
PLLELLKELLEELKQKLK

SEQ ID NO: 207 - protein, 18 aa, synthetic construct
  REGION 1..18: note = synthetic
PVLELLKELLEELKQKLK

SEQ ID NO: 208 - protein, 18 aa, synthetic construct
  REGION 1..18: note = synthetic
PALELFKDLLEELRQRLK

SEQ ID NO: 209 - protein, 18 aa, synthetic construct
  REGION 1..18: note = synthetic
PVLDLFRELLNELLQKLK

SEQ ID NO: 210 - protein, 18 aa, synthetic construct
  REGION 1..18: note = synthetic
PVLDLFRELLEELKQKLK

SEQ ID NO: 211 - protein, 18 aa, synthetic construct
  VARIANT 1..18: note = Xaa = Orn
PVLDLFRELLEELXQXLX

SEQ ID NO: 212 - protein, 18 aa, synthetic construct
  VARIANT 1..18: note = Xaa = Orn
PVLDLFXELLEELXQXLK

SEQ ID NO: 213 - protein, 18 aa, synthetic construct
  REGION 1..18: note = synthetic
PALELFKDLLEEFRQRLK

SEQ ID NO: 214 - protein, 18 aa, synthetic construct
  REGION 1..18: note = synthetic
PVLDLFRELLEELKQKLK

SEQ ID NO: 215 - protein, 18 aa, synthetic construct
  REGION 1..18: note = synthetic
PVLDLFRELLEEWKQKLK

SEQ ID NO: 216 - protein, 18 aa, synthetic construct
  REGION 1..18: note = synthetic
PVLELFKELLEELKQKLK

SEQ ID NO: 217 - protein, 18 aa, synthetic construct
  REGION 1..18: note = synthetic
PVLDLFRELLELLKQKLK

SEQ ID NO: 218 - protein, 18 aa, synthetic construct
  REGION 1..18: note = synthetic
PVLDLFRELLNELLQKLK

SEQ ID NO: 219 - protein, 18 aa, synthetic construct
  REGION 1..18: note = synthetic
PVLDLFRELLNELWQKLK

SEQ ID NO: 220 - protein, 18 aa, synthetic construct
  REGION 1..18: note = synthetic
PVLDLFRELLEELQKKLK

SEQ ID NO: 221 - protein, 18 aa, synthetic construct
  REGION 1..18: note = synthetic
DVLDLFRELLEELKQKLK

SEQ ID NO: 222 - protein, 18 aa, synthetic construct
  REGION 1..18: note = synthetic
PVLDAFRELLEALLQLKK

SEQ ID NO: 223 - protein, 18 aa, synthetic construct
  REGION 1..18: note = synthetic
PVLDAFRELLEALAQLKK

SEQ ID NO: 224 - protein, 18 aa, synthetic construct
  REGION 1..18: note = synthetic
PVLDLFREGWEELKQKLK

SEQ ID NO: 225 - protein, 18 aa, synthetic construct
  REGION 1..18: note = synthetic
PVLDAFRELAEALAQLKK

SEQ ID NO: 226 - protein, 18 aa, synthetic construct
  REGION 1..18: note = synthetic
PVLDAFRELGEALLQLKK

SEQ ID NO: 227 - protein, 18 aa, synthetic construct
  REGION 1..18: note = synthetic
PVLDLFRELGEELKQKLK

SEQ ID NO: 228 - protein, 18 aa, synthetic construct
  REGION 1..18: note = synthetic
PVLDLFREGLEELKQKLK

SEQ ID NO: 229 - protein, 18 aa, synthetic construct
  REGION 1..18: note = synthetic
PVLDLFRELLEEGKQKLK

SEQ ID NO: 230 - protein, 18 aa, synthetic construct
  REGION 1..18: note = synthetic
PVLELFERLLEDLQKKLK

SEQ ID NO: 231 - protein, 18 aa, synthetic construct
  REGION 1..18: note = synthetic
PVLDLFRELLEKLEQKLK

SEQ ID NO: 232 - protein, 18 aa, synthetic construct
  REGION 1..18: note = synthetic
PLLELFKELLEELKQKLK

SEQ ID NO: 233 - protein, 18 aa, synthetic construct
  REGION 1..18: note = synthetic
LDDLLQKWAEAFNQLLKK

SEQ ID NO: 234 - protein, 18 aa, synthetic construct
  REGION 1..18: note = synthetic
EWLKAFYEKVLEKLKELF

SEQ ID NO: 235 - protein, 18 aa, synthetic construct
  REGION 1..18: note = synthetic
EWLEAFYKKVLEKLKELF

SEQ ID NO: 236 - protein, 18 aa, synthetic construct
  REGION 1..18: note = synthetic
DWLKAFYDKVAEKLKEAF

SEQ ID NO: 237 - protein, 18 aa, synthetic construct
  REGION 1..18: note = synthetic
DWFKAFYDKVFEKFKEFF

SEQ ID NO: 238 - protein, 18 aa, synthetic construct
  REGION 1..18: note = synthetic
GIKKFLGSIWKFIKAFVG

SEQ ID NO: 239 - protein, 18 aa, synthetic construct
  REGION 1..18: note = synthetic
DWFKAFYDKVAEKFKEAF

SEQ ID NO: 240 - protein, 18 aa, synthetic construct
  REGION 1..18: note = synthetic
DWLKAFYDKVAEKLKEAF

SEQ ID NO: 241 - protein, 18 aa, synthetic construct
  REGION 1..18: note = synthetic
DWLKAFYDKVFEKFKEFF

SEQ ID NO: 242 - protein, 18 aa, synthetic construct
  REGION 1..18: note = synthetic
EWLEAFYKKVLEKLKELP

SEQ ID NO: 243 - protein, 18 aa, synthetic construct
  REGION 1..18: note = synthetic
DWFKAFYDKFFEKFKEFF

SEQ ID NO: 244 - protein, 18 aa, synthetic construct
  REGION 1..18: note = synthetic
EWLKAFYEKVLEKLKELF

SEQ ID NO: 245 - protein, 18 aa, synthetic construct
  REGION 1..18: note = synthetic
EWLKAEYEKVEEKLKELF

SEQ ID NO: 246 - protein, 18 aa, synthetic construct
  REGION 1..18: note = synthetic
EWLKAEYEKVLEKLKELF

SEQ ID NO: 247 - protein, 18 aa, synthetic construct
  REGION 1..18: note = synthetic
EWLKAFYKKVLEKLKELF

SEQ ID NO: 248 - protein, 15 aa, synthetic construct
  REGION 1..15: note = synthetic
PVLDLFRELLEQKLK

SEQ ID NO: 249 - protein, 16 aa, synthetic construct
  REGION 1..16: note = synthetic
PVLDLFRELLEELKQK

SEQ ID NO: 250 - protein, 16 aa, synthetic construct
  REGION 1..16: note = synthetic
PVLDLFRELLEKLKQK

SEQ ID NO: 251 - protein, 15 aa, synthetic construct
  REGION 1..15: note = synthetic
PVLDLFRELLEKLQK

SEQ ID NO: 252 - protein, 16 aa, synthetic construct
  REGION 1..16: note = synthetic
PVLDLFRELLEALKQK

SEQ ID NO: 253 - protein, 16 aa, synthetic construct
  REGION 1..16: note = synthetic
PVLDLFENLLERLKQK

SEQ ID NO: 254 - protein, 16 aa, synthetic construct
  REGION 1..16: note = synthetic
PVLDLFRELLNELKQK

SEQ ID NO: 255 - protein, 18 aa, synthetic construct
  REGION 1..18: note = synthetic
DWLKAFYDKVAEKLKEAF

SEQ ID NO: 256 - protein, 18 aa, synthetic construct
  REGION 1..18: note = synthetic
DWLKAFYDKVAEKLKEAF

SEQ ID NO: 257 - protein, 18 aa, synthetic construct
  REGION 1..18: note = synthetic
DWLKAFYDKVAEKLKEAF

SEQ ID NO: 258 - protein, 18 aa, synthetic construct
  REGION 1..18: note = synthetic
DWFKAFYDKVAEKLKEAF

SEQ ID NO: 259 - protein, 18 aa, synthetic construct
  REGION 1..18: note = synthetic
DWLKAFYDKVAEKFKEAF

SEQ ID NO: 260 - protein, 18 aa, synthetic construct
  REGION 1..18: note = synthetic
DWFKAFYDKVAEKFKEAF

SEQ ID NO: 261 - protein, 18 aa, synthetic construct
  REGION 1..18: note = synthetic
DWLKAFYDKVFEKFKEFF

SEQ ID NO: 262 - protein, 18 aa, synthetic construct
  REGION 1..18: note = synthetic
DWLKAFYDKFFEKFKEFF

SEQ ID NO: 263 - protein, 18 aa, synthetic construct
  REGION 1..18: note = synthetic
DWFKAFYDKFFEKFKEFF

SEQ ID NO: 264 - protein, 18 aa, synthetic construct
  REGION 1..18: note = synthetic
DWLKAFYDKVAEKLKEFF

SEQ ID NO: 265 - protein, 18 aa, synthetic construct
  REGION 1..18: note = synthetic
DWLKAFYDKVFEKFKEAF

SEQ ID NO: 266 - protein, 18 aa, synthetic construct
  REGION 1..18: note = synthetic
DWLKAFYDKVFEKLKEFF

SEQ ID NO: 267 - protein, 18 aa, synthetic construct
  REGION 1..18: note = synthetic
DWLKAFYDKVAEKFKEFF

SEQ ID NO: 268 - protein, 18 aa, synthetic construct
  REGION 1..18: note = synthetic
DWLKAFYDKVFEKFKEFF

SEQ ID NO: 269 - protein, 18 aa, synthetic construct
  REGION 1..18: note = synthetic
EWLKLFYEKVLEKFKEAF

SEQ ID NO: 270 - protein, 18 aa, synthetic construct
  REGION 1..18: note = synthetic
EWLKAFYDKVAEKFKEAF

SEQ ID NO: 271 - protein, 18 aa, synthetic construct
  REGION 1..18: note = synthetic
EWLKAFYDKVAEKLKEFF

SEQ ID NO: 272 - protein, 18 aa, synthetic construct
  REGION 1..18: note = synthetic
EWLKAFYDKVFEKFKEAF

SEQ ID NO: 273 - protein, 18 aa, synthetic construct
  REGION 1..18: note = synthetic
EWLKAFYDKVFEKLKEFF

SEQ ID NO: 274 - protein, 18 aa, synthetic construct
  REGION 1..18: note = synthetic
EWLKAFYDKVAEKFKEFF

SEQ ID NO: 275 - protein, 18 aa, synthetic construct
  REGION 1..18: note = synthetic
EWLKAFYDKVFEKFKEFF

SEQ ID NO: 276 - protein, 14 aa, synthetic construct
  REGION 1..14: note = synthetic
AFYDKVAEKLKEAF

SEQ ID NO: 277 - protein, 14 aa, synthetic construct
  REGION 1..14: note = synthetic
AFYDKVAEKFKEAF

SEQ ID NO: 278 - protein, 14 aa, synthetic construct
  REGION 1..14: note = synthetic
AFYDKVAEKFKEAF

SEQ ID NO: 279 - protein, 14 aa, synthetic construct
  REGION 1..14: note = synthetic
AFYDKFFEKFKEFF

SEQ ID NO: 280 - protein, 14 aa, synthetic construct
  REGION 1..14: note = synthetic
AFYDKFFEKFKEFF

SEQ ID NO: 281 - protein, 14 aa, synthetic construct
  REGION 1..14: note = synthetic
AFYDKVAEKFKEAF

SEQ ID NO: 282 - protein, 14 aa, synthetic construct
  REGION 1..14: note = synthetic
AFYDKVAEKLKEFF

SEQ ID NO: 283 - protein, 14 aa, synthetic construct
  REGION 1..14: note = synthetic
AFYDKVFEKFKEAF

SEQ ID NO: 284 - protein, 14 aa, synthetic construct
  REGION 1..14: note = synthetic
AFYDKVFEKLKEFF

SEQ ID NO: 285 - protein, 14 aa, synthetic construct
  REGION 1..14: note = synthetic
AFYDKVAEKFKEFF

SEQ ID NO: 286 - protein, 14 aa, synthetic construct
  REGION 1..14: note = synthetic
KAFYDKVFEKFKEF

SEQ ID NO: 287 - protein, 14 aa, synthetic construct
  REGION 1..14: note = synthetic
LFYEKVLEKFKEAF

SEQ ID NO: 288 - protein, 14 aa, synthetic construct
  REGION 1..14: note = synthetic
AFYDKVAEKFKEAF

SEQ ID NO: 289 - protein, 14 aa, synthetic construct
  REGION 1..14: note = synthetic
AFYDKVAEKLKEFF

SEQ ID NO: 290 - protein, 14 aa, synthetic construct
  REGION 1..14: note = synthetic
AFYDKVFEKFKEAF

SEQ ID NO: 291 - protein, 14 aa, synthetic construct
  REGION 1..14: note = synthetic
AFYDKVFEKLKEFF

SEQ ID NO: 292 - protein, 14 aa, synthetic construct
  REGION 1..14: note = synthetic
AFYDKVAEKFKEFF

SEQ ID NO: 293 - protein, 14 aa, synthetic construct
  REGION 1..14: note = synthetic
AFYDKVFEKFKEFF

SEQ ID NO: 294 - protein, 18 aa, synthetic construct
  REGION 1..18: note = synthetic
DWLKALYDKVAEKLKEAL

SEQ ID NO: 295 - protein, 18 aa, synthetic construct
  REGION 1..18: note = synthetic
DWFKAFYEKVAEKLKEFF

SEQ ID NO: 296 - protein, 18 aa, synthetic construct
  REGION 1..18: note = synthetic
DWFKAFYEKFFEKFKEFF

SEQ ID NO: 297 - protein, 18 aa, synthetic construct
  REGION 1..18: note = synthetic
EWLKALYEKVAEKLKEAL

SEQ ID NO: 298 - protein, 18 aa, synthetic construct
  REGION 1..18: note = synthetic
EWLKAFYEKVAEKLKEAF

SEQ ID NO: 299 - protein, 18 aa, synthetic construct
  REGION 1..18: note = synthetic
EWFKAFYEKVAEKLKEFF

SEQ ID NO: 300 - protein, 18 aa, synthetic construct
  REGION 1..18: note = synthetic
EWLKAFYEKVFEKFKEFF

SEQ ID NO: 301 - protein, 18 aa, synthetic construct
  REGION 1..18: note = synthetic
EWLKAFYEKFFEKFKEFF

SEQ ID NO: 302 - protein, 18 aa, synthetic construct
  REGION 1..18: note = synthetic
EWFKAFYEKFFEKFKEFF

SEQ ID NO: 303 - protein, 18 aa, synthetic construct
  REGION 1..18: note = synthetic
DFLKAWYDKVAEKLKEAW

SEQ ID NO: 304 - protein, 18 aa, synthetic construct
  REGION 1..18: note = synthetic
EFLKAWYEKVAEKLKEAW

SEQ ID NO: 305 - protein, 18 aa, synthetic construct
  REGION 1..18: note = synthetic
DFWKAWYDKVAEKLKEWW

SEQ ID NO: 306 - protein, 18 aa, synthetic construct
  REGION 1..18: note = synthetic
EFWKAWYEKVAEKLKEWW

SEQ ID NO: 307 - protein, 18 aa, synthetic construct
  REGION 1..18: note = synthetic
DKLKAFYDKVFEWAKEAF

SEQ ID NO: 308 - protein, 18 aa, synthetic construct
  REGION 1..18: note = synthetic
DKWKAVYDKFAEAFKEFL

SEQ ID NO: 309 - protein, 18 aa, synthetic construct
  REGION 1..18: note = synthetic
EKLKAFYEKVFEWAKEAF

SEQ ID NO: 310 - protein, 18 aa, synthetic construct
  REGION 1..18: note = synthetic
EKWKAVYEKFAEAFKEFL

SEQ ID NO: 311 - protein, 18 aa, synthetic construct
  REGION 1..18: note = synthetic
DWLKAFVDKFAEKFKEAY

SEQ ID NO: 312 - protein, 18 aa, synthetic construct
  REGION 1..18: note = synthetic
EKWKAVYEKFAEAFKEFL

SEQ ID NO: 313 - protein, 18 aa, synthetic construct
  REGION 1..18: note = synthetic
DWLKAFVYDKVFKLKEFF

SEQ ID NO: 314 - protein, 18 aa, synthetic construct
  REGION 1..18: note = synthetic
EWLKAFVYEKVFKLKEFF

SEQ ID NO: 315 - protein, 18 aa, synthetic construct
  REGION 1..18: note = synthetic
DWLRAFYDKVAEKLKEAF

SEQ ID NO: 316 - protein, 18 aa, synthetic construct
  REGION 1..18: note = synthetic
EWLRAFYEKVAEKLKEAF

SEQ ID NO: 317 - protein, 18 aa, synthetic construct
  REGION 1..18: note = synthetic
DWLKAFYDRVAEKLKEAF

SEQ ID NO: 318 - protein, 18 aa, synthetic construct
  REGION 1..18: note = synthetic
EWLKAFYERVAEKLKEAF

SEQ ID NO: 319 - protein, 18 aa, synthetic construct
  REGION 1..18: note = synthetic
DWLKAFYDKVAERLKEAF

SEQ ID NO: 320 - protein, 18 aa, synthetic construct
  REGION 1..18: note = synthetic
EWLKAFYEKVAERLKEAF

SEQ ID NO: 321 - protein, 18 aa, synthetic construct
  REGION 1..18: note = synthetic
DWLKAFYDKVAEKLREAF

SEQ ID NO: 322 - protein, 18 aa, synthetic construct
  REGION 1..18: note = synthetic
EWLKAFYEKVAEKLREAF

SEQ ID NO: 323 - protein, 18 aa, synthetic construct
  REGION 1..18: note = synthetic
DWLKAFYDRVAERLKEAF

SEQ ID NO: 324 - protein, 18 aa, synthetic construct
  REGION 1..18: note = synthetic
EWLKAFYERVAERLKEAF

SEQ ID NO: 325 - protein, 18 aa, synthetic construct
  REGION 1..18: note = synthetic
DWLRAFYDKVAEKLREAF

SEQ ID NO: 326 - protein, 18 aa, synthetic construct
  REGION 1..18: note = synthetic
EWLRAFYEKVAEKLREAF

SEQ ID NO: 327 - protein, 18 aa, synthetic construct
  REGION 1..18: note = synthetic
DWLRAFYDRVAEKLKEAF

SEQ ID NO: 328 - protein, 18 aa, synthetic construct
  REGION 1..18: note = synthetic
EWLRAFYERVAEKLKEAF

SEQ ID NO: 329 - protein, 18 aa, synthetic construct
  REGION 1..18: note = synthetic
DWLKAFYDKVAERLREAF

SEQ ID NO: 330 - protein, 18 aa, synthetic construct
  REGION 1..18: note = synthetic
EWLKAFYEKVAERLREAF

SEQ ID NO: 331 - protein, 18 aa, synthetic construct
  REGION 1..18: note = synthetic
DWLRAFYDKVAERLKEAF

SEQ ID NO: 332 - protein, 18 aa, synthetic construct
  REGION 1..18: note = synthetic
EWLRAFYEKVAERLKEAF

SEQ ID NO: 333 - protein, 18 aa, synthetic construct
  REGION 1..18: note = synthetic
FAEKFKEAVKDYFAKFWD

SEQ ID NO: 334 - protein, 18 aa, synthetic construct
  REGION 1..18: note = synthetic
DWFKAFYDKVAEKFKEAF

SEQ ID NO: 335 - protein, 18 aa, synthetic construct
  REGION 1..18: note = synthetic
DWLKAFYDKVAEKLKEAF

SEQ ID NO: 336 - protein, 34 aa, synthetic construct
  REGION 1..34: note = synthetic
PALEDLRQGLLPVLESFKVFLSALEEYTKKLNTQ

SEQ ID NO: 337 - DNA, 23 bp, synthetic construct
  source 1..23: mol_type = other DNA
gccagtatgcaagggagctcatg

SEQ ID NO: 338 - protein, 25 aa, synthetic construct
  REGION 1..25: note = synthetic
GASTAMGATCANCARGGEGCLCAMG

SEQ ID NO: 339 - DNA, 23 bp, synthetic construct
  source 1..23: mol_type = other DNA
gccagtatgcaatggagctcatg

SEQ ID NO: 340 - protein, 25 aa, synthetic construct
GASTAMGATCANCAMGGEGCLCATG

SEQ ID NO: 341 - protein, 9 aa, synthetic construct
ASMTNMELM

SEQ ID NO: 342 - protein, 18 aa, synthetic construct
  REGION 1..18: note = ApoA-I mimetic peptide
FWLKAFYDKVAEKLKEAF

SEQ ID NO: 343 - protein, 18 aa, synthetic construct
  REGION 1..18: note = ApoA-I mimetic peptide
DFLKAFYDKVAEKLKEAF

SEQ ID NO: 344 - protein, 18 aa, synthetic construct
  REGION 1..18: note = ApoA-I mimetic peptide
DWFRAFYDKVAEKFREAF

SEQ ID NO: 345 - protein, 18 aa, synthetic construct
  REGION 1..18: note = ApoA-I mimetic peptide
DWFKAFYDRVAERFKEAF

SEQ ID NO: 346 - protein, 18 aa, synthetic construct
  REGION 1..18: note = ApoA-I mimetic peptide
  MOD_RES 4: note = Xaa is L-2,4-diaminobutyric acid
  MOD_RES 9: note = Xaa is L-2,4-diaminobutyric acid
  MOD_RES 13: note = Xaa is L-2,4-diaminobutyric acid
  MOD_RES 15: note = Xaa is L-2,4-diaminobutyric acid
DWLXAFYDXVAEXLXEAF

SEQ ID NO: 347 - protein, 18 aa, Homo sapiens
MKAAVLTLAVLFLTGSQA

SEQ ID NO: 348 - protein, 6 aa, Homo sapiens
RHFWQQ

SEQ ID NO: 349 - protein, 243 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ